(19) United States
(12) Patent Application Publication
(10) Pub. No.: US 2025/0101027 A1
(43) Pub. Date: Mar. 27, 2025

(54) ORGANIC COMPOUND, LIGHT-EMITTING DEVICE, LIGHT-EMITTING APPARATUS, AND ELECTRONIC APPLIANCE

(71) Applicant: SEMICONDUCTOR ENERGY LABORATORY CO., LTD., Atsugi-shi (JP)

(72) Inventors: Yuko KUBOTA, Atsugi (JP); Yui YOSHIYASU, Atsugi (JP); Sachiko KAWAKAMI, Atsugi (JP); Nobuharu OHSAWA, Zama (JP); Shinya FUKUZAKI, Atsugi (JP); Toshiki SASAKI, Kawasaki (JP)

(21) Appl. No.: 18/885,879

(22) Filed: Sep. 16, 2024

(30) Foreign Application Priority Data
Sep. 22, 2023 (JP) 2023-158365
Aug. 9, 2024 (JP) 2024-134216

(51) Int. Cl. C07D 487/04; H10K 50/17; H10K 85/60

(52) U.S. Cl. CPC C07D 487/04; H10K 50/171; H10K 85/654; H10K 85/6572

(57) ABSTRACT

An organic compound with an electron-transport property and low water solubility is provided. An organic compound represented by General Formula (G1-1) below is provided. At least any one of R² to R⁹ is a group represented by General Formula (R-1) or (R-2); each of the other groups of R² to R⁹ independently represents hydrogen, an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms; α¹ represents a substituted or unsubstituted arylene group having 6 to 30 carbon atoms; n represents 1 or 2; R¹¹ to R²⁶ each independently represent hydrogen (including deuterium) or an alkyl group having 1 to 10 carbon atoms; and p and q each independently represent 0 or 1.

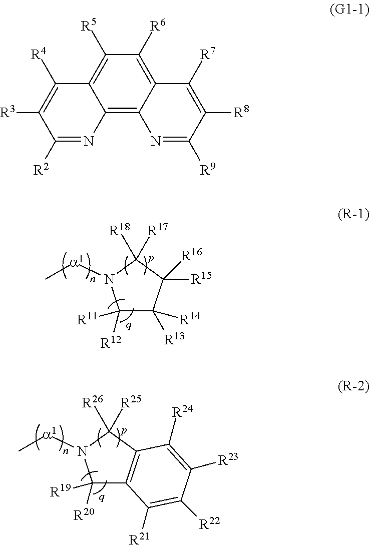

### BACKGROUND OF THE INVENTION

### 1. Field of the Invention

One embodiment of the present invention relates to an organic compound, a light-emitting device, a light-emitting apparatus, a light-emitting and light-receiving apparatus, a display apparatus, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display apparatus, a liquid crystal display apparatus, a light-emitting apparatus, a lighting device, a power storage device, a memory device, an image capturing device, a driving method thereof, and a manufacturing method thereof.

### 2. Description of the Related Art

Recently, display apparatuses have been expected to be applied to a variety of uses. Usage examples of large-sized display apparatuses include a television device for home use (also referred to as TV or television receiver), digital signage, and a public information display (PID). In addition, a smartphone and a tablet terminal each including a touch panel, for example, are being developed as portable information terminals.

Higher-resolution display apparatuses have been required. For example, devices for virtual reality (VR), augmented reality (A R), substitutional reality (SR), or mixed reality (MR) are given as devices requiring high-resolution display apparatuses and have been actively developed.

Light-emitting apparatuses that include light-emitting devices (also referred to as light-emitting elements) have been developed as display apparatuses, for example. Light-emitting devices utilizing electroluminescence (hereinafter referred to as EL; such devices are also referred to as EL devices or EL elements) have features such as ease of reduction in thickness and weight, high-speed response to input signals, and driving with a constant DC voltage power source, and have been used in display apparatuses.

Patent Document 1 discloses a display apparatus for VR that includes an organic EL device (also referred to as organic EL element). Patent Document 2 discloses a light-emitting device that has a low driving voltage and high reliability and includes an electron-injection layer formed using a mixed film of a transition metal and an organic compound having an unshared electron pair.

### REFERENCES

### Patent Documents

[Patent Document 1] International Publication No. WO2018/087625
[Patent Document 2] Japanese Published Patent Application No. 2018-201012

### SUMMARY OF THE INVENTION

As a method for forming an organic compound layer in a predetermined shape, a vacuum evaporation method with a metal mask (mask vapor deposition) is widely used. However, in these days of higher density and higher resolution, mask vapor deposition has come close to the limit of increasing the resolution for various reasons such as the alignment accuracy and the distance between the mask and the substrate. By contrast, a finer pattern can be formed by shape processing of an organic compound layer by a lithography method. Moreover, because of the ease of large-area processing in this method, the processing of an organic compound layer by a lithography method is being researched.

At the time of processing an organic compound layer by a lithography method, if the water solubility of an organic compound used for the organic compound layer is high, the organic compound layer is dissolved when exposed to water or a chemical solution containing water as a solvent, which might cause degraded characteristics, a shape defect, or the like. In addition, if the glass transition temperature (T_{g}) of the organic compound used for the organic compound layer is low, the organic compound layer is crystallized in a heating step for removing water adsorbed onto the organic compound layer after being exposed to water or a chemical solution containing water as a solvent, which might reduce film quality.

An object of one embodiment of the present invention is to provide an organic compound with an electron-transport property. Another object of one embodiment of the present invention is to provide an organic compound with low water solubility. Another object of one embodiment of the present invention is to provide an organic compound having a high T_{g}. Another object of one embodiment of the present invention is to provide a light-emitting device with favorable characteristics. Another object of one embodiment of the present invention is to provide a novel organic compound or a novel light-emitting device.

An object of one embodiment of the present invention is to provide a light-emitting apparatus with high display quality. Another object of one embodiment of the present invention is to provide a high-resolution light-emitting apparatus. Another object of one embodiment of the present invention is to provide a high-definition light-emitting apparatus. Another object of one embodiment of the present invention is to provide a light-emitting apparatus having a high aperture ratio. Another object of one embodiment of the present invention is to provide a highly reliable light-emitting apparatus. Another object of one embodiment of the present invention is to provide a novel light-emitting apparatus that is highly convenient, useful, or reliable. Another object of one embodiment of the present invention is to provide a novel electronic appliance that is highly convenient, useful, or reliable. Another object of one embodiment of the present invention is to provide a novel lighting device that is highly convenient, useful, or reliable. Another object of one embodiment of the present invention is to provide a novel light-emitting apparatus, a novel electronic appliance, or a novel lighting device.

Note that the description of these objects does not preclude the existence of other objects. One embodiment of the present invention does not need to achieve all of these objects. Other objects will be apparent from and can be derived from the description of the specification, the drawings, the claims, and the like.

One embodiment of the present invention is an organic compound represented by General Formula (G1-1).

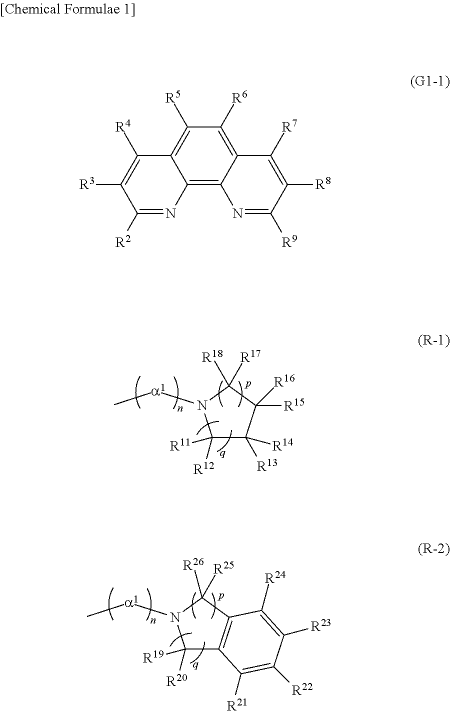

In General Formula (G1-1), at least any one of R² to R⁹ is a group represented by General Formula (R-1) or (R-2), and each of the other groups of R² to R⁹ independently represents hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms. In General Formulae (R-1) and (R-2), α¹ represents a substituted or unsubstituted arylene group having 6 to 30 carbon atoms; n represents 1 or 2; in the case where n is 2, a plurality of substituted or unsubstituted arylene groups having 6 to 30 carbon atoms may be the same or different from each other; R¹¹ to R²⁶ each independently represent hydrogen (including deuterium) or an alkyl group having 1 to 10 carbon atoms; andp and q each independently represent 0 or 1. Any two of R¹¹ to R¹⁸ may be bonded to each other to form a ring; any two of R¹⁹ to R²⁶ may be bonded to each other to form a ring; and in the case where two or more of R² to R⁹ are groups represented by General Formula (R-1) or (R-2), the two or more of R² to R⁹ may be the same or different from each other.

In the organic compound with the above structure, one or both of R⁴ and R⁷ are further preferably groups represented by General Formula (R-1) or (R-2).

Another embodiment of the present invention is an organic compound represented by General Formula (G1-2).

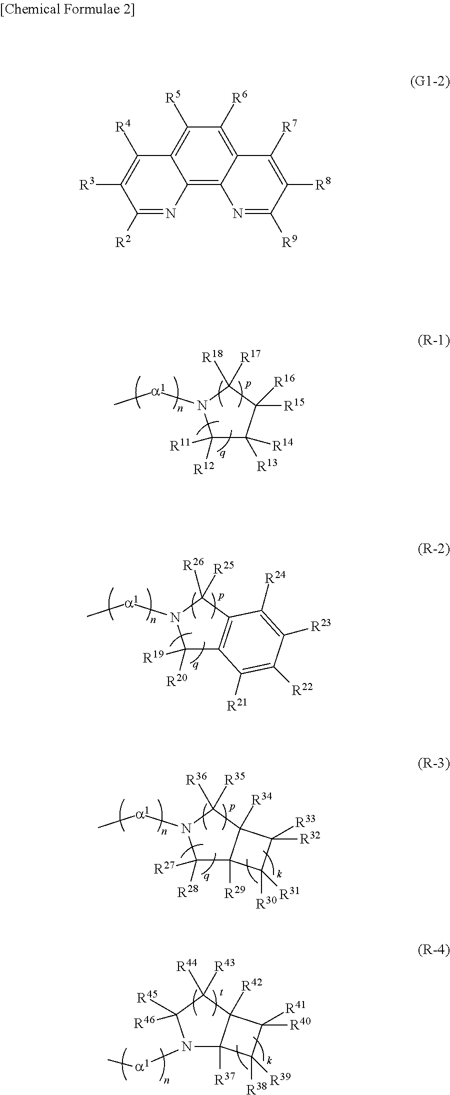

In General Formula (G1-2), at least any one of R² to R⁹ is a group represented by any of General Formulae (R-1) to (R-4), and each of the other groups of R² to R⁹ independently represents hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms. In General Formulae (R-1) to (R-4), α¹ represents a substituted or unsubstituted arylene group having 6 to 30 carbon atoms; n represents 1 or 2; in the case where n is 2, a plurality of substituted or unsubstituted arylene groups having 6 to 30 carbon atoms may be the same or different from each other; R¹¹ to R⁴⁶ each independently represent hydrogen (including deuterium) or an alkyl group having 1 to 10 carbon atoms; p and q each independently represent 0 or 1; k represents an integer of 0 to 4; and t represents 0 or 1. Any two of R¹¹ to R¹⁸ may be bonded to each other to form a ring; any two of R¹⁹ to R²⁶ may be bonded to each other to form a ring; any two of R²⁷ to R³⁶ may be bonded to each other to form a ring; any two of R³⁷ to R⁴⁶ may be bonded to each other to form a ring; and in the case where two or more of R² to R⁹ are groups represented by any of General Formulae (R-1) to (R-4), the two or more of R² to R⁹ may be the same or different from each other.

In the organic compound with the above structure, one or both of R⁴ and R⁷ are further preferably groups represented by any of General Formulae (R-1) to (R-4).

Another embodiment of the present invention is an organic compound represented by General Formula (G1-3).

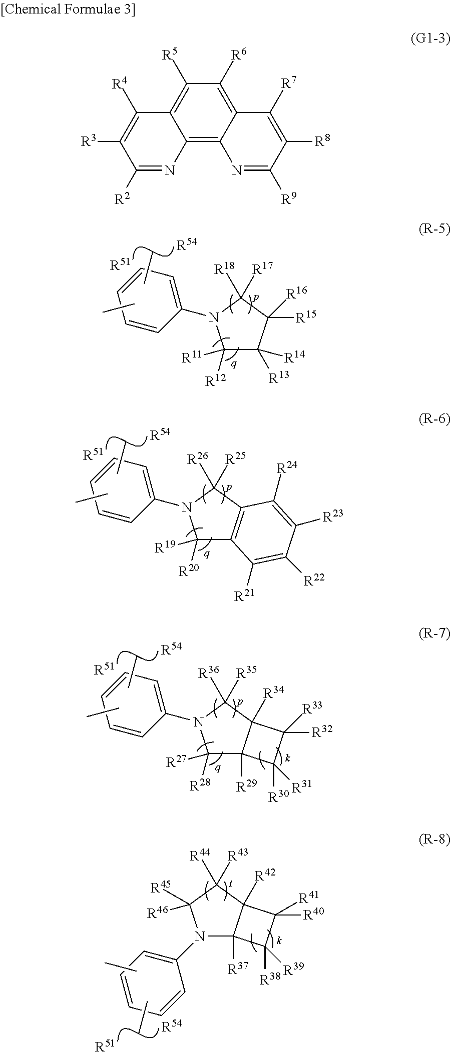

In General Formula (G1-3), at least any one of R² to R⁹ is a group represented by any of General Formulae (R-5) to (R-8), and each of the other groups of R² to R⁹ independently represents hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms. In General Formulae (R-5) to (R-8), R⁵¹ to R⁵⁴ each independently represent hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms; R¹¹ to R⁴⁶ each independently represent hydrogen (including deuterium) or an alkyl group having 1 to 10 carbon atoms; p and q each independently represent 0 or 1; k represents an integer of 0 to 4; and t represents 0 or 1. Any two of R¹¹ to R¹⁸ may be bonded to each other to form a ring; any two of R¹⁹ to R²⁶ may be bonded to each other to form a ring; any two of R²⁷ to R³⁶ may be bonded to each other to form a ring; any two of R³⁷ to R⁴⁶ may be bonded to each other to form a ring; and in the case where two or more of R² to R⁹ are groups represented by any of General Formulae (R-5) to (R-8), the two or more of R² to R⁹ may be the same or different from each other.

In the organic compound with the above structure, one or both of R⁴ and R⁷ are further preferably groups represented by any of General Formulae (R-5) to (R-8).

Another embodiment of the present invention is an organic compound represented by General Formula (G1-4),

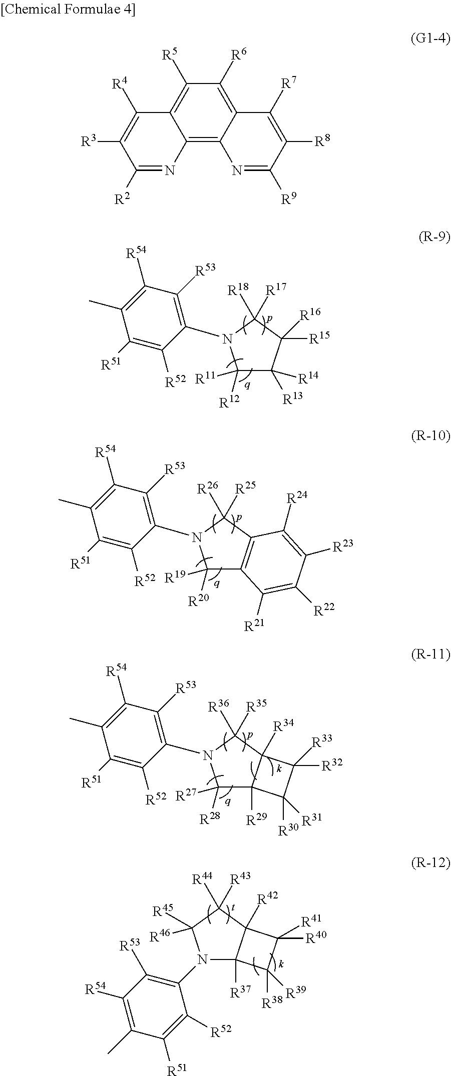

In General Formula (G1-4), at least any one of R² to R⁹ is a group represented by any of General Formulae (R-9) to (R-12), and each of the other groups of R² to R⁹ independently represents hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms. In General Formulae (R-9) to (R-12), R⁵¹ to Rs⁴ each independently represent hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms; R¹¹ to R⁴⁶ each independently represent hydrogen (including deuterium) or an alkyl group having 1 to 10 carbon atoms; p and q each independently represent 0 or 1; k represents an integer of 0 to 4; and t represents 0 or 1. Any two of R¹¹ to R¹⁸ may be bonded to each other to form a ring; any two of R¹⁹ to R²⁶ may be bonded to each other to form a ring; any two of R²⁷ to R³⁶ may be bonded to each other to form a ring; any two of R³⁷ to R⁴⁶ may be bonded to each other to form a ring; and in the case where two or more of R² to R⁹ are groups represented by any of General Formulae (R-9) to (R-12), the two or more of R² to R⁹ may be the same or different from each other.

In the organic compound with the above structure, one or both of R⁴ and R⁷ are further preferably groups represented by any of General Formulae (R-5) to (R-8).

Another embodiment of the present invention is an organic compound represented by General Formula (G2).

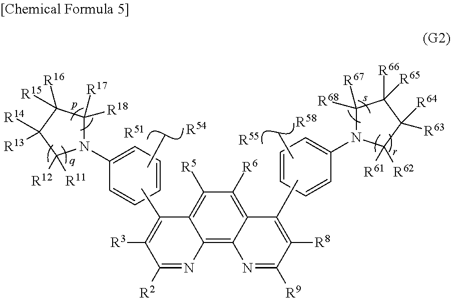

In General Formula (G2), R², R³, R⁵, R⁶, R⁸, R⁹, and R⁵¹ to R⁵⁸ each independently represent hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms; R¹¹ to R¹⁸ and R⁶¹ to R⁶⁸ each independently represent hydrogen (including deuterium) or an alkyl group having 1 to 10 carbon atoms; and p, q, r, and s each independently represent 0 or 1.

Another embodiment of the present invention is an organic compound represented by General Formula (G3).

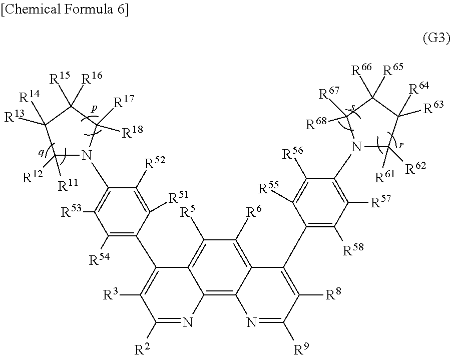

In General Formula (G3), R², R³, R⁵, R⁶, R⁸, R⁹, and R⁵¹ to R⁵⁸ each independently represent hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms; R¹¹ to R¹⁸ and R⁶¹ to R⁶⁸ each independently represent hydrogen (including deuterium) or an alkyl group having 1 to 10 carbon atoms; and p, q, and s each independently represent 0 or 1.

Another embodiment of the present invention is an organic compound represented by General Formula (G4).

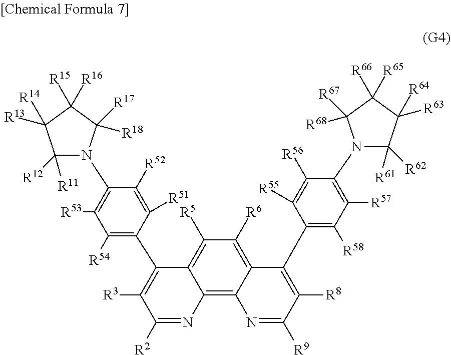

In General Formula (G4), R², R³, R⁵, R⁶, R⁸, R⁹, and R⁵¹ to R⁵⁸ each independently represent hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms; and R¹¹ to R¹⁸ and R⁶¹ to R⁶⁸ each independently represent hydrogen (including deuterium) or an alkyl group having 1 to 10 carbon atoms.

Another embodiment of the present invention is the organic compound with any of the above structures in which a group that is bonded at a 4-position of a phenanthroline ring and a group that is bonded at a 7-position of the phenanthroline ring are the same.

Another embodiment of the present invention is an organic compound represented by Structural Formula (100) or Structural Formula (152).

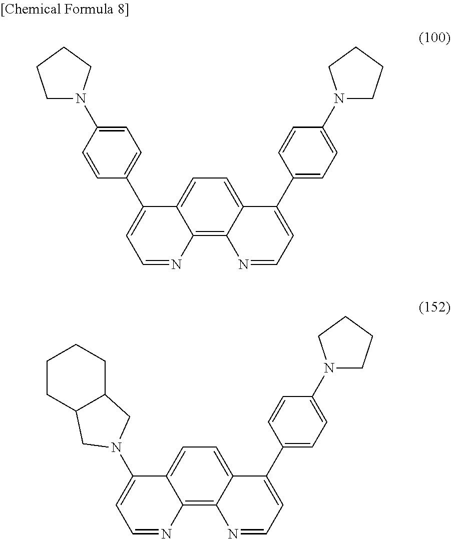 [SMILES: c1cc(-c2ccc(N3CCCC3)cc2)c2ccc3c(-c4ccc(N5CCCC5)cc4)ccnc3c2n1 ; c1cc(-c2ccc(N3CCCC3)cc2)c2ccc3c(N4CC5CCCCC5C4)ccnc3c2n1]

Another embodiment of the present invention is a light-emitting device including the organic compound with any of the above structures.

Another embodiment of the present invention is a light-emitting device including a first electrode, a second electrode, and an organic compound layer; the organic compound layer is between the first electrode and the second electrode; the organic compound layer includes a light-emitting layer and an electron-injection layer; the electron-injection layer is between the light-emitting layer and the second electrode; and the electron-injection layer includes the organic compound with any of the above structures.

Another embodiment of the present invention is a light-emitting device including a first electrode, a second electrode, and an organic compound layer; the organic compound layer is between the first electrode and the second electrode; the organic compound layer includes an intermediate layer, a first light-emitting layer, and a second light-emitting layer; the intermediate layer is between the first light-emitting layer and the second light-emitting layer; and the intermediate layer includes the organic compound with any of the above structures.

Another embodiment of the present invention is a light-emitting apparatus including the light-emitting device with the above structure, and a transistor or a substrate.

Another embodiment of the present invention is an electronic appliance including the light-emitting apparatus with the above structure, and a detection portion, an input portion, or a communication portion.

Note that the light-emitting apparatus in this specification includes, in its category, a display apparatus and an image display device that use a light-emitting device. The light-emitting apparatus may also include a module in which a light-emitting device over a substrate is provided with a connector such as an anisotropic conductive film or a tape carrier package (TCP), a module in which a printed wiring board is further provided at the end of the TCP, and a module in which an integrated circuit (IC) is directly mounted on a light-emitting device by a chip on glass (COG) method. Furthermore, a lighting device or the like may include the light-emitting apparatus.

One embodiment can provide an organic compound with an electron-transport property. Another embodiment can provide an organic compound with low water solubility. Another embodiment can provide an organic compound having a high T_{g}. Another embodiment can provide a light-emitting device with favorable characteristics. Another embodiment can provide a novel organic compound or a novel light-emitting device.

One embodiment can provide a light-emitting apparatus with high display quality. Another embodiment can provide a high-resolution light-emitting apparatus. Another embodiment can provide a high-definition light-emitting apparatus. Another embodiment can provide alight-emitting apparatus having a high aperture ratio. Another embodiment can provide a highly reliable light-emitting apparatus. Another embodiment can provide a novel light-emitting apparatus that is highly convenient, useful, or reliable. Another embodiment can provide a novel display module that is highly convenient, useful, or reliable. Another embodiment can provide a novel electronic appliance that is highly convenient, useful, or reliable. Another embodiment can provide a novel light-emitting apparatus, a novel display module, a novel electronic appliance, or a novel semiconductor device.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

In the accompanying drawings:

FIGS. 1A and 1B show light-emitting devices;

FIGS. 2A to 2D each show a light-emitting device;

FIGS. 3A and 3B are, respectively, a top view and a cross-sectional view of a light-emitting apparatus;

FIGS. 4A to 4D each show a light-emitting device;

FIGS. 5A to 5E are cross-sectional views showing an example of a method for manufacturing a display apparatus;

FIGS. 6A to 6D are cross-sectional views showing an example of a method for manufacturing a display apparatus;

FIGS. 7A to 7D are cross-sectional views showing an example of a method for manufacturing a display apparatus;

FIGS. 8A to 8C are cross-sectional views showing an example of a method for manufacturing a display apparatus;

FIGS. 9A to 9C are cross-sectional views showing an example of a method for manufacturing a display apparatus;

FIGS. 10A to 10C are cross-sectional views showing an example of a method for manufacturing a display apparatus;

FIGS. 11A and 11B are perspective views showing a structure example of a display module;

FIGS. 12A and 12B are cross-sectional views showing structure examples of a display apparatus;

FIGS. 13A to 13D show examples of electronic appliances;

FIGS. 14A to 14F show examples of electronic appliances;

FIG. 15 shows a ¹H NMR spectrum of PrdP2Phen;

FIG. 16 is a graph showing luminance-current density characteristics of a light-emitting device I and a comparative light-emitting device 2;

FIG. 17 is a graph showing luminance-voltage characteristics of the light-emitting device I and the comparative light-emitting device 2;

FIG. 18 is a graph showing current efficiency-current density characteristics of the light-emitting device 1 and the comparative light-emitting device 2;

FIG. 19 is a graph showing current density-voltage characteristics of the light-emitting device 1 and the comparative light-emitting device 2;

FIG. 20 is a graph showing electroluminescence spectra of the light-emitting device 1 and the comparative light-emitting device 2;

FIG. 21 is a graph showing luminance-current density characteristics of a light-emitting device 3 and a light-emitting device 4;

FIG. 22 is a graph showing luminance-voltage characteristics of the light-emitting device 3 and the light-emitting device 4;

FIG. 23 is a graph showing current efficiency-current density characteristics of the light-emitting device 3 and the light-emitting device 4;

FIG. 24 is a graph showing current density-voltage characteristics of the light-emitting device 3 and the light-emitting device 4;

FIG. 25 is a graph showing electroluminescence spectra of the light-emitting device 3 and the light-emitting device 4;

FIGS. 26A to 26C are optical micrographs of the light-emitting device 3, the light-emitting device 4, and a comparative light-emitting device 5;

FIG. 27 shows a ¹H NMR spectrum of Hid-PrdPPhen;

FIG. 28 is a graph showing luminance-current density characteristics of a light-emitting device 6 and a light-emitting device 7;

FIG. 29 is a graph showing luminance-voltage characteristics of the light-emitting device 6 and the light-emitting device 7;

FIG. 30 is a graph showing current efficiency-current density characteristics of the light-emitting device 6 and the light-emitting device 7;

FIG. 31 is a graph showing current density-voltage characteristics of the light-emitting device 6 and the light-emitting device 7;

FIG. 32 is a graph showing electroluminescence spectra of the light-emitting device 6 and the light-emitting device 7;

FIG. 33 is a graph showing luminance-current density characteristics of a light-emitting device 8 and a comparative light-emitting device 9;

FIG. 34 is a graph showing luminance-voltage characteristics of the light-emitting device 8 and the comparative light-emitting device 9;

FIG. 35 is a graph showing current efficiency-current density characteristics of the light-emitting device 8 and the comparative light-emitting device 9;

FIG. 36 is a graph showing current density-voltage characteristics of the light-emitting device 8 and the comparative light-emitting device 9;

FIG. 37 is a graph showing electroluminescence spectra of the light-emitting device 8 and the comparative light-emitting device 9; and

FIGS. 38A and 38B are optical micrographs of the light-emitting device 8 and the comparative light-emitting device 9.

### DETAILED DESCRIPTION OF THE INVENTION

Embodiments will be described in detail with reference to the drawings. Note that the embodiments of the present invention are not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

Note that in structures of the invention described below, the same portions or portions having similar functions are denoted by the same reference numerals in different drawings, and the description thereof is not repeated. The same hatching pattern is used for portions having similar functions, and the portions are not denoted by specific reference numerals in some cases.

The position, size, range, or the like of each component illustrated in drawings does not represent the actual position, size, range, or the like in some cases for easy understanding. Therefore, the disclosed invention is not necessarily limited to the position, size, range, or the like disclosed in the drawings.

Note that the terms “film” and “layer” can be used interchangeably depending on the case or the circumstances. For example, the term “conductive layer” can be replaced with the term “conductive film”. For another example, the term “insulating film” can be replaced with the term “insulating layer”.

In this specification and the like, a device formed using a metal mask or a fine metal mask (FMM) is sometimes referred to as a device having a metal mask (MM) structure. In this specification and the like, a device formed without using a metal mask or an FMM is sometimes referred to as a device having a metal maskless (MMI) structure.

In this specification and the like, a hole or an electron is sometimes referred to as a carrier. Specifically, a hole-injection layer or an electron-injection layer may be referred to as a carrier-injection layer, a hole-transport layer or an electron-transport layer may be referred to as a carrier-transport layer, and a hole-blocking layer or an electron-blocking layer may be referred to as a carrier-blocking layer. One layer may have two or three functions of the carrier-injection layer, the carrier-transport layer, and the carrier-blocking layer in some cases.

In this specification and the like, a light-emitting device (also referred to as a light-emitting element) includes an EL layer between a pair of electrodes. The EL layer includes at least a light-emitting layer. In this specification and the like, one of the pair of electrodes may be referred to as a pixel electrode and the other may be referred to as a common electrode.

In this specification and the like, a tapered shape indicates a shape in which at least part of a side surface of a component is inclined to a substrate surface. For example, a tapered shape preferably includes a region where the angle formed by the inclined side surface and the substrate surface (such an angle is also referred to as a taper angle) is less than 90°. Note that the side surface of the component and the substrate surface are not necessarily completely flat, and may have a substantially planar shape with a small curvature or slight unevenness.

Note that the light-emitting apparatus in this specification includes, in its category, a display apparatus and an image display device that use an organic EL device. The light-emitting apparatus may also include a module in which an organic EL device is provided with a connector such as an anisotropic conductive film or a tape carrier package (TCP), a module in which a printed wiring board is further provided at the end of the TCP, and a module in which an integrated circuit (IC) is directly mounted on an organic EL device by a chip on glass (COG) method. Furthermore, a lighting device or the like may include the light-emitting apparatus.

### Embodiment 1

In this embodiment, an organic compound of one embodiment of the present invention will be described.

The organic compound of one embodiment of the present invention is represented by General Formula (G1-1).

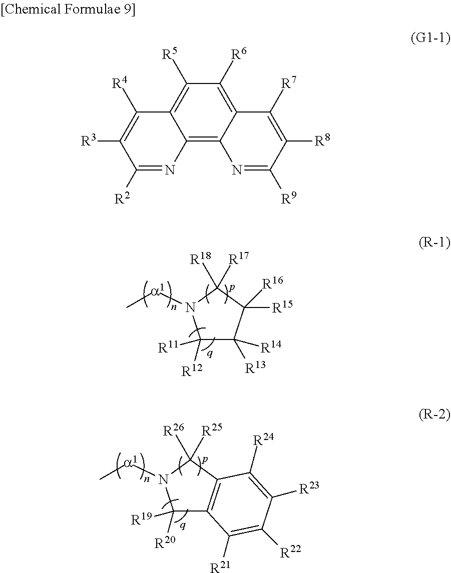

In General Formula (G1-1), at least any one of R² to R⁹ is a group represented by General Formula (R-1) or (R-2), and each of the other groups of R² to R⁹ independently represents hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms. In General Formulae (R-1) and (R-2), α¹ represents a substituted or unsubstituted arylene group having 6 to 30 carbon atoms; n represents 1 or 2; in the case where n is 2, a plurality of substituted or unsubstituted arylene groups having 6 to 30 carbon atoms may be the same or different from each other; R¹¹ to R²⁶ each independently represent hydrogen (including deuterium) or an alkyl group having 1 to 10 carbon atoms; and p and q each independently represent 0 or 1. Any two of R¹¹ to R¹⁸ may be bonded to each other to form a ring; any two of R¹⁹ to R²⁶ may be bonded to each other to form a ring; and in the case where two or more of R² to R⁹ are groups represented by General Formula (R-1) or (R-2), the two or more of R² to R⁹ may be the same or different from each other.

The organic compound represented by General Formula (G1-1) includes a 1,10-phenanthroline ring and thus has an electron-transport property. The 1,10-phenanthroline ring can be coordinated to a metal since its nitrogen atoms (N) at the 1- and 10-positions have unshared electron pairs. Accordingly, in the case where the organic compound represented by General Formula (G1-1) is mixed with a metal, a coordinate bond is formed due to electron donation from two N atoms in the 1,10-phenanthroline ring to the metal, whereby a stabilized complex can be formed and the electron density of the metal can be increased. In the case where a layer including the organic compound represented by General Formula (G1-1) and a metal layer such as an electrode are stacked, a coordinate bond is formed due to electron donation from two N atoms in the 1,10-phenanthroline ring to a metal included in the metal layer, whereby the electron density of the metal included in the metal layer can be increased.

The organic compound represented by General Formula (G1-1) includes a cyclic alkylamino group having greater than or equal to 3 and less than or equal to 5 ring members. Since the cyclic alkylamino group has a high electron-donating property, the electron density of the 1,10-phenanthroline ring can be increased, so that a property of donating electrons from the two N atoms in the 1,10-phenanthroline ring to a metal can be increased and a stable coordinate bond can be formed. The number of ring members of the cyclic alkylamino group is preferably less than or equal to 5 because the electron density of a nitrogen atom of the cyclic alkylamino group is higher than that of the case where the number of ring members of the cyclic alkylamino group is greater than or equal to 6, in which case the electron-donating property can be increased.

When the electron density of the 1,10-phenanthroline ring increases, an acid dissociation constant pKₐ increases. Although the acid dissociation constant pKₐ that is excessively high results in high water solubility, the organic compound represented by General Formula (G1-1) has a structure in which the 1,10-phenanthroline ring and the cyclic alkylamino group are bonded to each other with a substituted or unsubstituted arylene group having 6 to 30 carbon atoms therebetween. Accordingly, a property of donating electrons from the cyclic alkylamino group to the 1,10-phenanthroline ring decreases as compared with the case where the 1,10-phenanthroline ring and the cyclic alkylamino group are directly connected; thus, the electron density of the 1,10-phenanthroline ring can be inhibited from being excessively high. Accordingly, it is possible to prevent the acid dissociation constant pKₐ from being excessively high, so that an increase in water solubility can be inhibited. The organic compound represented by General Formula (G1-1) has a larger molecular weight and a smaller volume proportion of the cyclic alkylamino group, which is a hydrophilic group, to the whole molecular structure than the organic compound in which the 1,10-phenanthroline ring and the cyclic alkylamino group are directly connected. It can be said that this also enables an increase in water solubility to be inhibited. Furthermore, a substituted or unsubstituted arylene group having 6 to 30 carbon atoms, which is a hydrophobic group, is preferably included because the molecular weight is increased, so that the organic compound with a higher T_{g} can be achieved.

Since the organic compound of one embodiment of the present invention has lower water solubility, using the organic compound in an organic compound layer of a light-emitting device whose fabrication process includes processing using water or a chemical solution containing water as a solvent (i.e., a light-emitting device involving processing by a lithography method) can inhibit the organic compound layer from being dissolved. Since the organic compound of one embodiment of the present invention has a higher T_{g}, when the organic compound is used in the organic compound layer, the organic compound layer can be inhibited from being crystallized in a heating step for removing water adsorbed onto the organic compound layer after a step of being exposed to water or a chemical solution containing water as a solvent. Thus, with use of the organic compound of one embodiment of the present invention, it is possible to fabricate a light-emitting device having favorable characteristics and in which defective light emission is inhibited.

Note that in this specification and the like, “hydrogen” includes protium and deuterium. Deuterium is a stable hydrogen isotope having a mass number of 2. Protium is a stable hydrogen isotope having a mass number of 1. The bond dissociation energy of a bond between carbon and deuterium is higher than that of a bond between carbon and protium, and thus is stable and not easily cut. Thus, in the case where the organic compound of one embodiment of the present invention includes deuterium, carbon-hydrogen bond dissociation is inhibited, allowing the compound to be stable.

The organic compound of one embodiment of the present invention is represented by General Formula (G1-2),

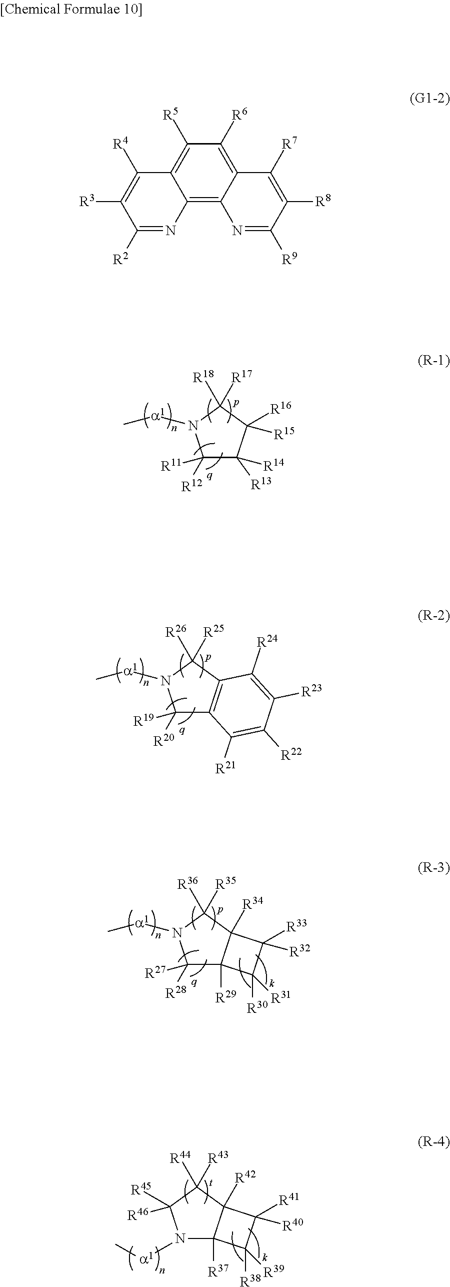

In General Formula (G1-2), at least any one of R² to R⁹ is a group represented by any of General Formulae (R-1) to (R-4), and each of the other groups of R² to R⁹ independently represents hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms. In General Formulae (R-1) to (R-4), α¹ represents a substituted or unsubstituted arylene group having 6 to 30 carbon atoms; n represents 1 or 2; in the case where n is 2, a plurality of substituted or unsubstituted arylene groups having 6 to 30 carbon atoms may be the same or different from each other; R¹¹ to R⁴⁶ each independently represent hydrogen (including deuterium) or an alkyl group having 1 to 10 carbon atoms; p and q each independently represent 0 or 1; k represents an integer of 0 to 4; and t represents 0 or 1. Any two of R¹¹ to R¹⁸ may be bonded to each other to form a ring; any two of R¹⁹ to R²⁶ may be bonded to each other to form a ring: any two of R²⁷ to R³⁶ may be bonded to each other to form a ring; any two of R³⁷ to R⁴⁶ may be bonded to each other to form a ring; and in the case where two or more of R² to R⁹ are groups represented by any of General Formulae (R-1) to (R-4), the two or more of R² to R⁹ may be the same or different from each other.

In General Formula (G1-2), General Formulae (R-3) and (R-4) are shown as examples of the case where any two of R¹¹ to R¹⁸ in General Formula (R-1) of General Formula (G1-1) are bonded to each other to form a ring. The ring formation in this manner is preferable because the organic compound having lower water solubility and a higher T_{g} than that of a monocyclic alkylamino group can be achieved.

The organic compound of one embodiment of the present invention is represented by General Formula (G1-3).

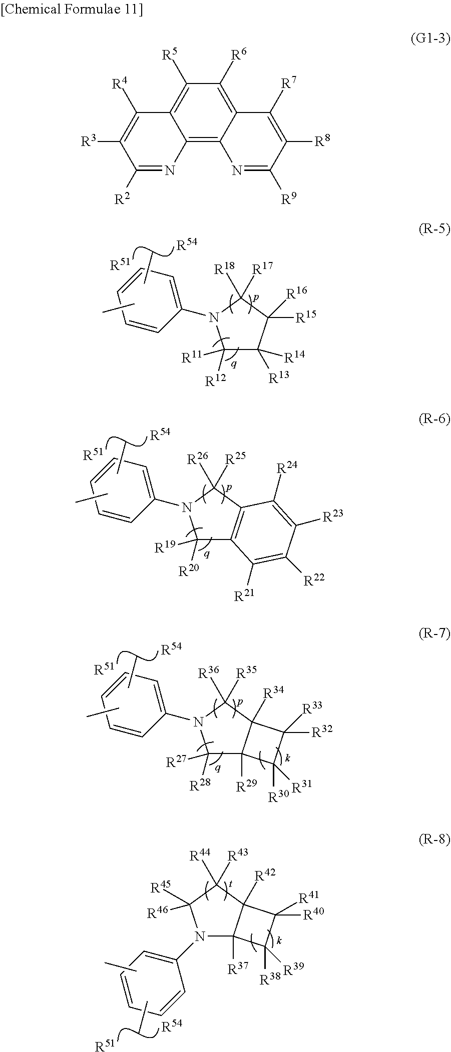

In General Formula (G1-3), at least any one of R² to R⁹ is a group represented by any of General Formulae (R-5) to (R-8), and each of the other groups of R² to R⁹ independently represents hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms. In General Formulae (R-5) to (R-8), R⁵¹ to R⁵⁴ each independently represent hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms; R¹¹ to R⁴⁶ each independently represent hydrogen (including deuterium) or an alkyl group having 1 to 10 carbon atoms; p and q each independently represent 0 or 1; k represents an integer of 0 to 4; and t represents 0 or 1. Any two of R¹¹ to R¹⁸ may be bonded to each other to form a ring; any two of R¹⁹ to R²⁶ may be bonded to each other to form a ring; any two of R²⁷ to R³⁶ may be bonded to each other to form a ring; any two of R³⁷ to R⁴⁶ may be bonded to each other to form a ring; and in the case where two or more of R² to R⁹ are groups represented by any of General Formulae (R-5) to (R-8), the two or more of R² to R⁹ may be the same or different from each other.

The organic compound represented by General Formula (G1-3) is different from the organic compound represented by General Formula (G1-2) in that α¹ is limited to a phenylene group. When α¹ is a phenylene group in this manner, the degree of expansion of the effective conjugation length by α¹ can be inhibited, so that absorption of light in the visible light region can be inhibited. The substitution site of the phenylene group is preferably at the para-position, in which case a property of donating electrons from the cyclic alkylamino group to nitrogen in the phenanthroline ring can be increased. Thus, with use of the organic compound represented by General Formula (G1-3), the emission efficiency of the light-emitting device can be increased. In the case where the substitution site of the phenylene group is at the meta-position, the heat resistance is lower than that of the case where the substitution site thereof is at the para-position. Since the property of donating electrons from the cyclic alkylamino group is lower in the meta-position than in the ortho-position or the para-position, the substitution site of the phenylene group is preferably at the para-position. In the case where the substitution site of the phenylene group is at the ortho-position, it is probable that the cyclic alkylamino group covers the periphery of the 1,10-phenanthroline ring, which might inhibit the coordinate bond between nitrogen in the 1,10-phenanthroline ring and the metal. The substitution position of the phenylene group is preferably at the para-position because such a steric concern can be resolved.

The organic compound of one embodiment of the present invention is represented by General Formula (G1-4).

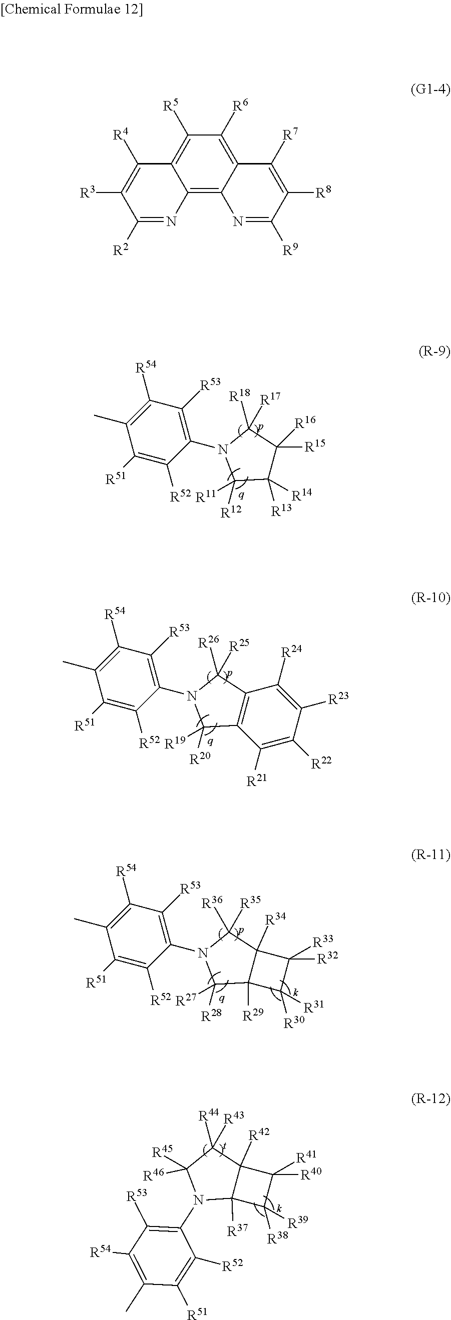

In General Formula (G1-4), at least any one of R² to R⁹ is a group represented by any of General Formulae (R-9) to (R-12), and each of the other groups of R² to R⁹ independently represents hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms. In General Formulae (R-9) to (R-12), R⁵¹ to R⁵⁴ each independently represent hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms; R¹¹ to R⁴⁶ each independently represent hydrogen (including deuterium) or an alkyl group having 1 to 10 carbon atoms; p and q each independently represent 0 or 1; k represents an integer of 0 to 4; and t represents 0 or 1. Any two of R¹¹ to R¹⁸ may be bonded to each other to form a ring: any two of R¹⁹ to R²⁶ may be bonded to each other to form a ring; any two of R²⁷ to R³⁶ may be bonded to each other to form a ring; any two of R³⁷ to R⁴⁶ may be bonded to each other to forn a ring; and in the case where two or more of R² to R⁹ are groups represented by any of General Formulae (R-9) to (R-12), the two or more of R² to R⁹ may be the same or different from each other.

The organic compound represented by General Formula (G1-4) is different from the organic compound represented by General Formula (G1-3) in that the substitution position of the phenylene group that connects the 1,10-phenanthroline ring to the cyclic alkylamino group is limited to the para-position. The use of such a p-phenylene group enables the p-phenylene groups to not be twisted with respect to the 1,10-phenanthroline rings and to be arranged on the same plane. Thus, the use of the p-phenylene group is preferable because the effect of the cyclic alkylamino group that increases the electron density of the 1,10-phenanthroline ring is less likely to be reduced.

In the organic compound represented by General Formula (G1-1), one of R⁴ and R⁷ is preferably a group represented by General Formula (R-1) or (R-2), and both of R⁴ and R⁷ are further preferably groups represented by General Formula (R-1) or (R-2). In the organic compound represented by General Formula (G1-2), one of R⁴ and R⁷ is preferably a group represented by any of General Formulae (R-1) to (R-4), and both of R⁴ and R⁷ are further preferably groups represented by any of General Formulae (R-1) to (R-4). In the organic compound represented by General Formula (G1-3), one of R⁴ and R⁷ is preferably a group represented by any of General Formulae (R-5) to (R-8), and both of R⁴ and R⁷ are further preferably groups represented by any of General Formulae (R-5) to (R-8). In the organic compound represented by General Formula (G1-4), one of R⁴ and R⁷ is preferably a group represented by any of General Formulae (R-9) to (R-12), and both of R⁴ and R⁷ are further preferably groups represented by any of General Formulae (R-9) to (R-12). Introducing the group with an electron-donating property to the 4- and 7-positions of the 1,10-phenanthroline ring in this manner can increase the electron density of the nitrogen atoms at the 1- and 10-positions, which are the para-positions with respect to the 4- and 7-positions. In addition, steric congestion around the nitrogen atoms at the 1- and 10-positions can be inhibited, and the electron density around the nitrogen atoms can be increased. Thus, the efficiency of the interaction between the organic compounds represented by General Formulae (G1-1) to (G1-4) and the metal can be increased.

The organic compound of one embodiment of the present invention is represented by General Formula (G2).

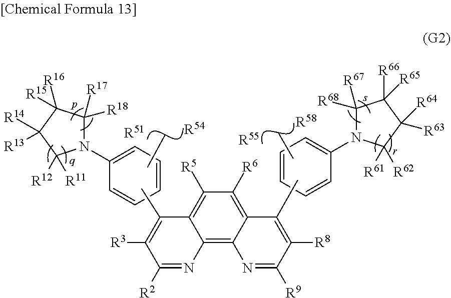

In General Formula (G2), R², R³, R⁵, R⁶, R⁸, R⁹, and R⁵¹ to R⁵⁸ each independently represent hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms; R¹¹ to R¹⁸ and R⁶¹ to R⁶⁸ each independently represent hydrogen (including deuterium) or an alkyl group having 1 to 10 carbon atoms; and p, q, r, and s each independently represent 0 or 1.

The organic compound represented by General Formula (G2) is different from that of General Formula (G1-3) in that both R⁴ and R⁷ are limited to groups represented by General Formula (R-5). When both R⁴ and R⁷ are groups represented by General Formula (R-5) in this manner, the electron density of the nitrogen atoms at the 1- and 10-positions of the phenanthroline ring can be increased. In addition, steric congestion around the nitrogen atoms at the 1- and 10-positions can be inhibited, and the electron density around the nitrogen atoms can be increased. Thus, the efficiency of the interaction between the organic compound represented by General Formula (G2) and the metal can be increased.

The organic compound of one embodiment of the present invention is represented by General Formula (G3).

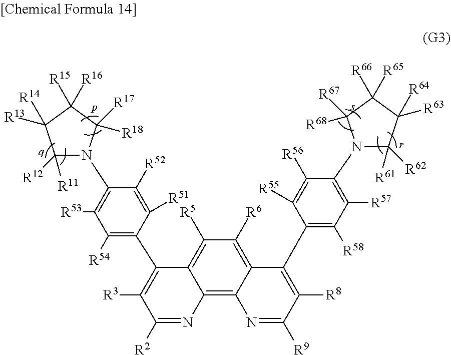

In General Formula (G3), R², R³, R⁵, R⁶, R⁸, R⁹, and R⁵¹ to R⁵⁸ each independently represent hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms; R¹¹ to R¹⁸ and R⁶¹ to R⁶⁸ each independently represent hydrogen (including deuterium) or an alkyl group having 1 to 10 carbon atoms; and p, q, and s each independently represent 0 or 1.

The organic compound represented by General Formula (G3) is different from the organic compound represented by General Formula (G2) in that the substitution position of the phenylene group that connects the 1,10-phenanthroline ring to the cyclic alkylamino group is limited to the para-position. The use of such a p-phenylene group enables the p-phenylene groups to not be twisted with respect to the 1,10-phenanthroline rings and to be arranged on the same plane. Thus, the use of the p-phenylene group is preferable because the effect of the cyclic alkylamino group that increases the electron density of the 1,10-phenanthroline ring is less likely to be reduced.

The organic compound of one embodiment of the present invention is represented by General Formula (G4).

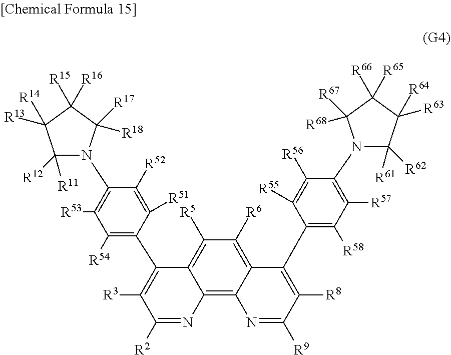

In General Formula (G4), R², R³, R⁵, R⁶, R⁸, R⁹, and R⁵¹ to R⁵⁸ each independently represent hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms; and R¹¹ to R¹⁸ and R⁶¹ to R⁶⁸ each independently represent hydrogen (including deuterium) or an alkyl group having 1 to 10 carbon atoms.

The organic compound represented by General Formula (G4) is different from the organic compound represented by General Formula (G3) in that the number of ring members of the cyclic alkylamino group is limited to five. Five-membered ring of the cyclic alkylamino group is preferable because the stability is increased as compared with the case where the number of ring members is less than or equal to four and the effect of increasing the electron density of the 1,10-phenanthroline ring is increased as compared with the case where the number of ring members is greater than or equal to six.

Next, specific examples of an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, an aryl group having 6 to 30 carbon atoms, a heteroaryl group having 1 to 30 carbon atoms, and an arylene group having 6 to 30 carbon atoms that can be used in General Formulae (G1-1) to (G1-4) and (G2) to (G4) will be described. Note that in the specific examples described below, some or all of hydrogen atoms may be deuterium. The groups that can be used in the above general formulae are not limited to the following specific examples.

### <<Specific Examples of Alkyl Group Having 1 to 10 Carbon Atoms>>

Specific examples of an alkyl group having 1 to 10 carbon atoms include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neo-pentyl group, a hexyl group, an isohexyl group, a sec-hexyl group, a tert-hexyl group, a neo-hexyl group, a 3-methylpentyl group, a 2-methylpentyl group, a 2-ethylbutyl group, a 1,2-dimethylbutyl group, a 2,3-dimethylbutyl group, and a 1-ethylhexyl group. Particularly in the case where a cyclic alkylamino group includes an alkyl group having 1 to 10 carbon atoms, the water solubility of the organic compound can be further reduced when a branched alkyl group such as a tert-butyl group is used. This is because the surface area of the hydrophilic alkylamino group is smaller than that in the case where a straight-chain alkyl group with the same molecular weight is used.

### <<Specific Examples of Cycloalkyl Group Having 3 to 10 Carbon Atoms>>

Specific examples of a cycloalkyl group having 3 to 10 carbon atoms include a cyclopropyl group, a cyclobutyl group, a methylcyclobutyl group, a cyclopentyl group, a methylcyclopentyl group, an isopropylcyclopentyl group, a tert-butylcyclopropyl group, a cyclohexyl group, a methylcyclohexyl group, a tert-butylcyclohexyl group, a cycloheptyl group, a methylcycloheptyl group, an isopropylcycloheptyl group, a cyclooctyl group, a methylcyclooctyl group, an isopropylcyclohexyl group, a cyclononyl group, a methylcyclononyl group, a cyclodecyl group, and an adamantyl group.

### <<Alkoxy Group Having 1 to 10 Carbon Atoms>>

Specific examples of an alkoxyl group having 1 to 10 carbon atoms include a methoxy group, an ethoxy group, an n-propoxy group, an isopropoxy group, an n-butoxy group, a sec-butoxy group, an isobutoxy group, a tert-butoxy group, an n-pentyloxy group, an isopentyloxy group, a sec-pentyloxy group, a tert-pentyloxy group, a neo-pentyloxy group, an n-hexyloxy group, an isohexyloxy group, a sec-hexyloxy group, a tert-hexyloxy group, a neo-hexyloxy group, and a cyclohexyloxy group.

### <<Cyclic Secondary Amino Group Having 2 to 10 Carbon Atoms>>

In this specification and the like, a cyclic secondary amino group having 2 to 10 carbon atoms refers to a monovalent group obtained by eliminating a hydrogen atom from a nitrogen atom of a cyclic secondary amine having 2 to 10 carbon atoms. Specific examples of the cyclic secondary amino group having 2 to 10 carbon atoms include a pyrrolidin-1-yl group, an isoindol-2-yl group, a dihydroisoindol-2-yl group, a tetrahydroisoindol-2-yl group, a hexahydroisoindol-2-yl group, an octahydro-1H-isoindol-2-yl group, a hexahydroisoindolin-2-yl group, a piperidin-1-yl group, an aziridin-1-yl group, an azetidin-1-yl group, an octahydrocyclopenta[c]pyrrol-2-yl group, an octahydro-4,7-methano-1H-isoindol-2-yl group, a 2-azabicyclo[3.1.0]hexan-2-yl group, a 3-azabicyclo[3.1.0]hexan-2-yl group, a 3-azabicyclo[3.2.0]heptan-2-yl group, a 5-azaspiro[3.4]octan-5-yl group, an 8-azabicyclo[3.2.1]octan-8-yl group, a 7-azabicyclo[2.2.1]heptan-7-yl group, a 5-azaspiro[2.4]heptan-5-yl group, and a 5-azabicyclo[2.1.1]hexan-5-yl group. In the case where the cyclic secondary amino group having 2 to 10 carbon atoms includes a substituent, a specific example of the substituent is an alkyl group having 1 to 10 carbon atoms.

Specific examples of the substituted or unsubstituted cyclic secondary amino group having 2 to 10 carbon atoms include groups represented by General Formulae (N-1) to (N-4) below. The groups represented by General Formulae (N-1) to (N-4) below are further preferable because of their high electron-donating properties.

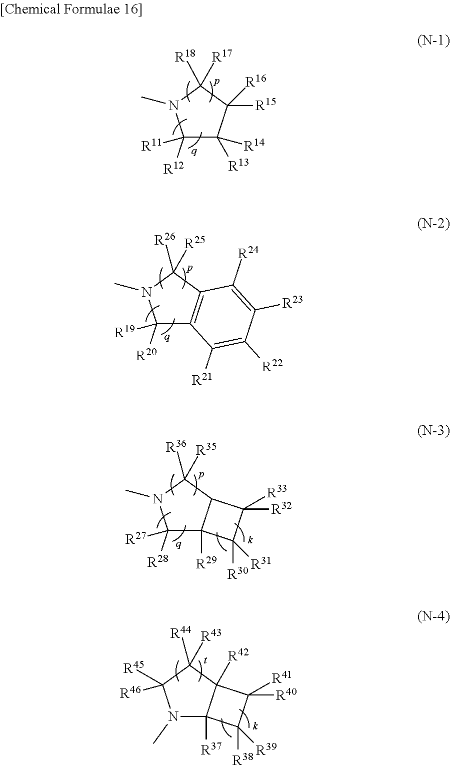

In General Formulae (N-1) to (N-4), R¹¹ to R⁴⁶ each independently represent hydrogen (including deuterium) or an alkyl group having 1 to 10 carbon atoms; p and q each independently represent 0 or 1; k represents an integer of 0 to 4; and t represents 0 or 1. Any two of R¹¹ to R¹⁸ may be bonded to each other to form a ring; any two of R¹⁹ to R²⁶ may be bonded to each other to form a ring; any two of R²⁷ to R³⁶ may be bonded to each other to form a ring; and any two of R³⁷ to R⁴⁶ may be bonded to each other to form a ring.

### <<Aryl Group Having 6 to 30 Carbon Atoms>>

Specific examples of an aryl group having 6 to 30 carbon atoms include a phenyl group, an o-tolyl group, an m-tolyl group, a p-tolyl group, a mesityl group, an o-biphenyl group, an m-biphenyl group, a p-biphenyl group, a 1-naphthyl group, a 2-naphthyl group, a fluorenyl group, a 9,9-dimethylfluorenyl group, a spirobifluorenyl group, a phenanthrenyl group, an anthracenyl group, and a fluoranthenyl group. In the case where the aryl group having 6 to 30 carbon atoms includes a substituent, examples of the substituent include an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, and a phenyl group.

### <<Arylene Group Having 6 to 30 Carbon Atoms>>

Specific examples of an arylene group having 6 to 30 carbon atoms include a phenylene group, a biphenyl-diyl group, a naphthalene-diyl group, a fluorenine-diyl group, an acenaphthene-diyl group, an anthracene-diyl group, a terphenyl-diyl group, a triphenylene-diyl group, a phenanthrene-diyl group, a tetracen-yl group, a benzanthracene-diyl group, a pyrene-diyl group, and a spirobi[9H-fluoren]-diyl group. In the case where the arylene group having 6 to 30 carbon atoms includes a substituent, examples of the substituent include an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, and a phenyl group.

### <<Heteroaryl Group Having 1 to 30 Carbon Atoms>>

In this specification and the like, a heteroaryl group having 1 to 30 carbon atoms refers to a monovalent group obtained by eliminating a hydrogen atom from carbon atoms forming a ring of a monocyclic or polycyclic heterocyclic aromatic compound having 1 to 30 carbon atoms, Specific examples of the heteroaryl group having 1 to 30 carbon atoms include a 1,3,5-triazin-2-yl group, a 1,2,4-triazin-3-yl group, a pyrimidin-4-yl group, a pyrazin-2-yl group, a 2-pyridyl group, a 3-pyridyl group, a 4-pyridyl group, a carbazolyl group, a dibenzofuranyl group, a dibenzothiophenyl group, a benzonaphthofuranyl group, a benzonaphthothiophenyl group, an indolocarbazolyl group, a benzofurocarbazolyl group, a benzothienocarbazolyl group, an indenocarbazolyl group, and a dibenzocarbazolyl group. In the case where the arylene group having 6 to 30 carbon atoms includes a substituent, examples of the substituent include an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, and a phenyl group.

The above examples of the substituent can be employed in the above general formulae.

Specific examples of the organic compound of one embodiment of the present invention represented by the above general formulae include organic compounds represented by Structural Formulae (100) to (156) below.

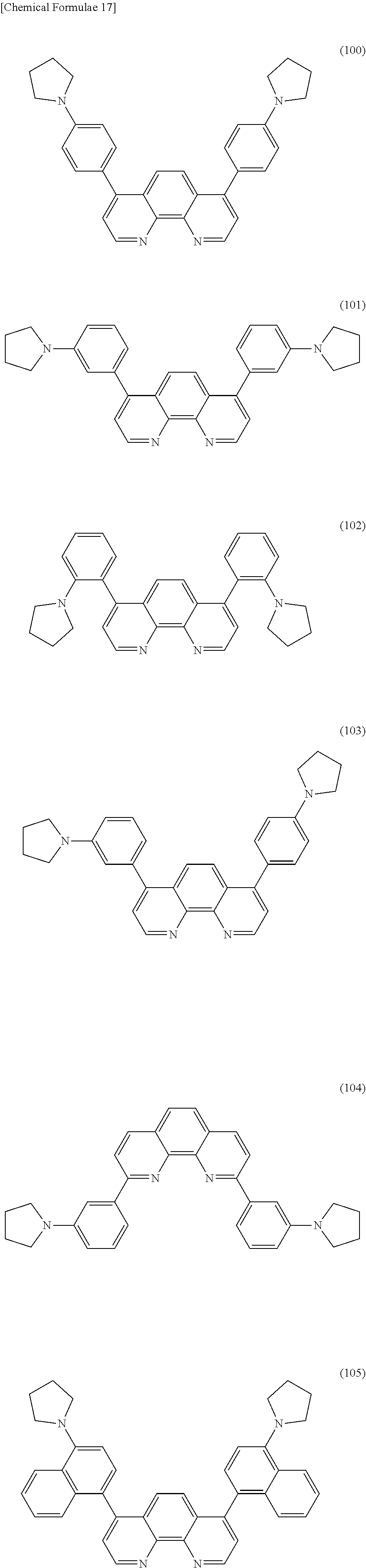

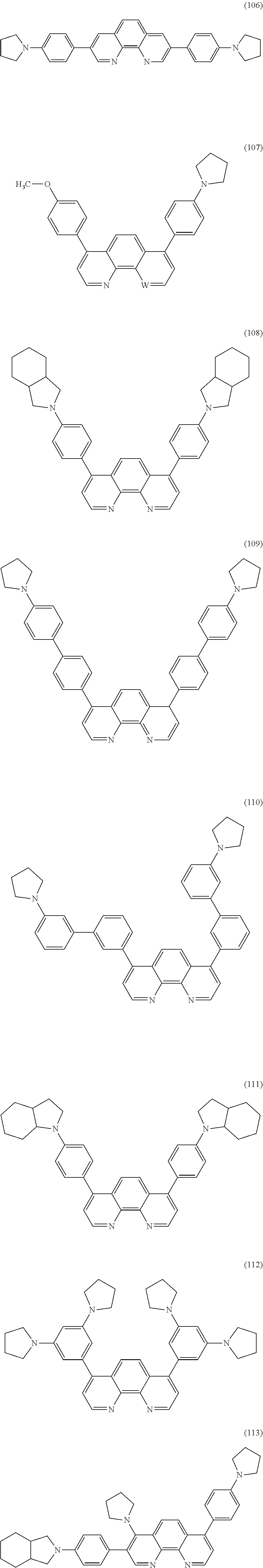

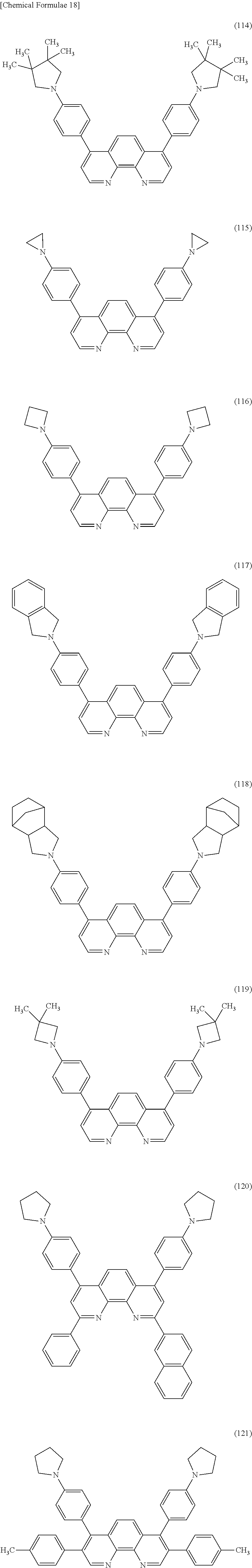

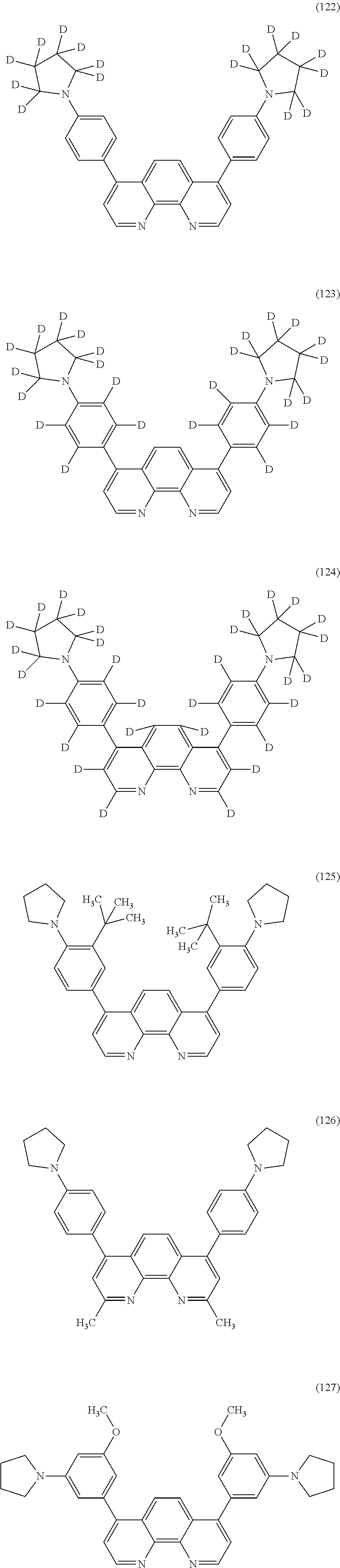

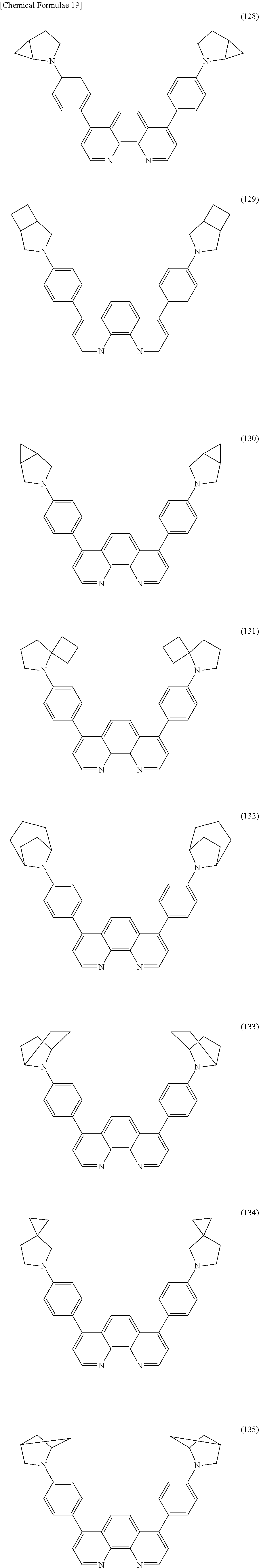

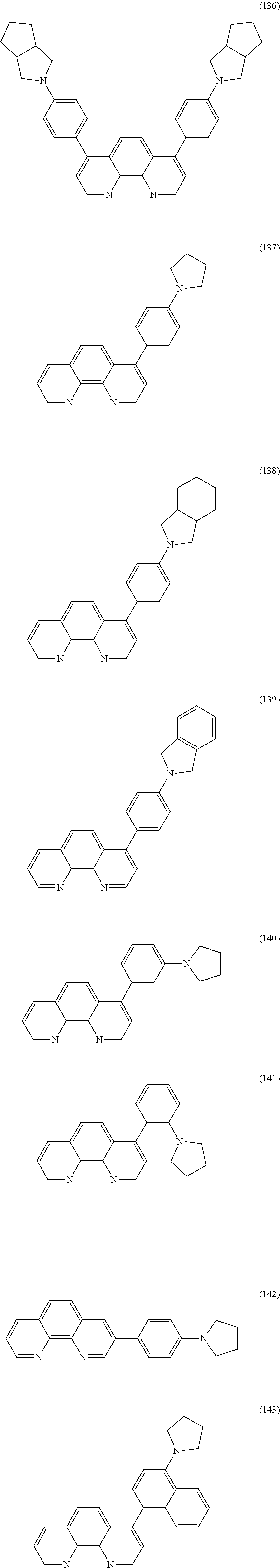

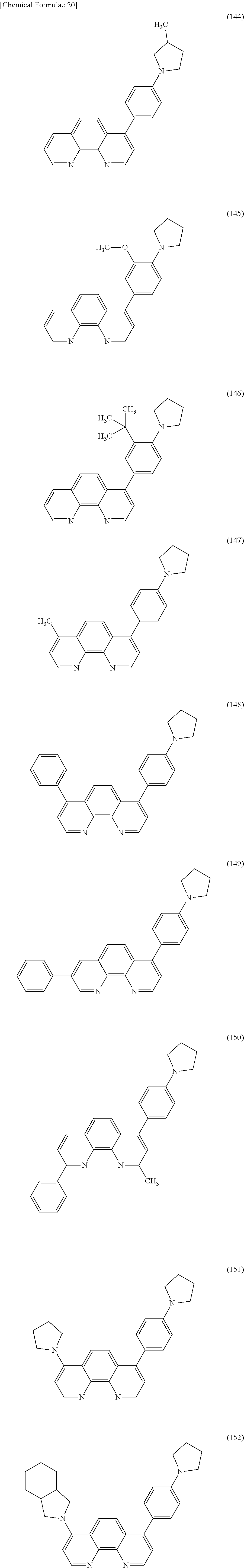

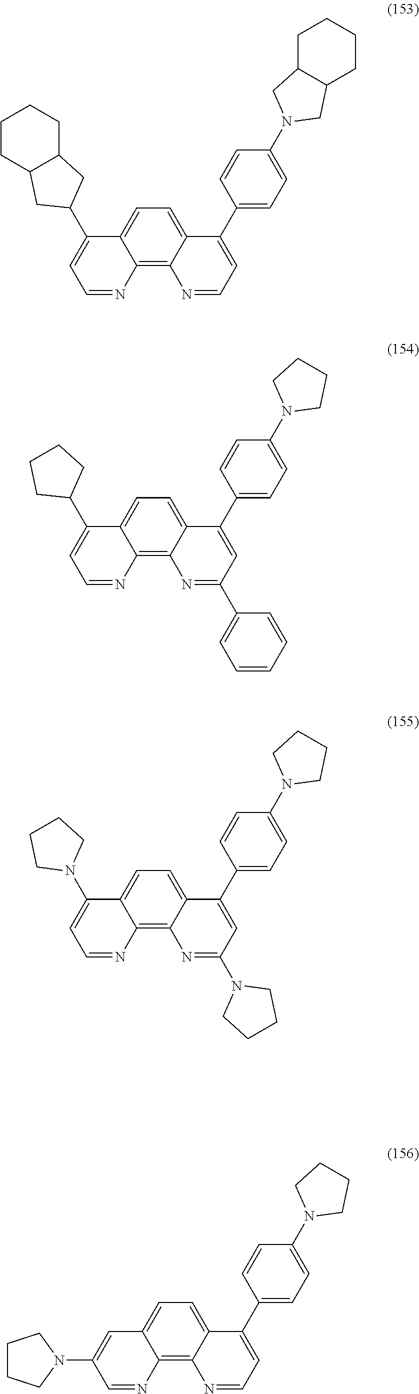

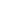

The organic compounds represented by Structural Formulae (100) to (156) are examples of the organic compound of one embodiment of the present invention; however, one embodiment of the present invention is not limited thereto.

Next, as an example of a synthesis method of the organic compound of one embodiment of the present invention, a synthesis method of the organic compound represented by General Formula (G1) below is described. Note that the synthesis method of the organic compound represented by General Formula (G1) can employ a variety of reactions and is not limited to the following synthesis methods.

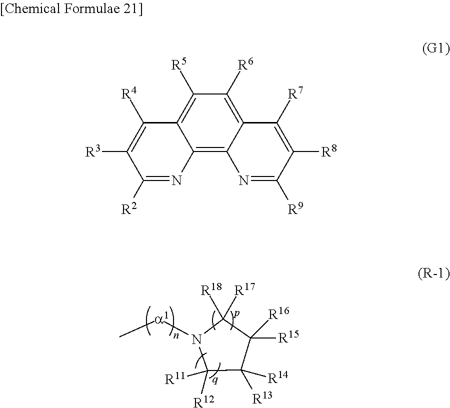

In General Formula (GI) above, at least any one of R² to R⁹ is a group represented by General Formula (R-1), and each of the other groups of R² to R⁹ independently represents hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms. In General Formula (R-1), α¹ represents a substituted or unsubstituted arylene group having 6 to 30 carbon atoms; n represents 1 or 2; in the case where n is 2, a plurality of substituted or unsubstituted arylene groups having 6 to 30 carbon atoms may be the same or different from each other: R¹¹ to R²⁶ each independently represent hydrogen (including deuterium) or an alkyl group having 1 to 10 carbon atoms; and p and q each independently represent 0 or 1. Any two of R¹¹ to R¹⁸ may be bonded to each other to form a ring, and in the case where two or more of R² to R⁹ are groups represented by General Formula (R-1), the two or more of R² to R⁹ may be the same or different from each other.

As shown in Synthesis Scheme (A-1) below, a compound (a1) having a halogen compound or a triflate group of a 1,10-phenanthroline derivative and a compound (a2) in which boronic acid is bonded to a group represented by General Formula (R-1) are coupled by the Suzuki-Miyaura reaction, whereby the organic compound represented by General Formula (G1) of one embodiment of the present invention can be obtained.

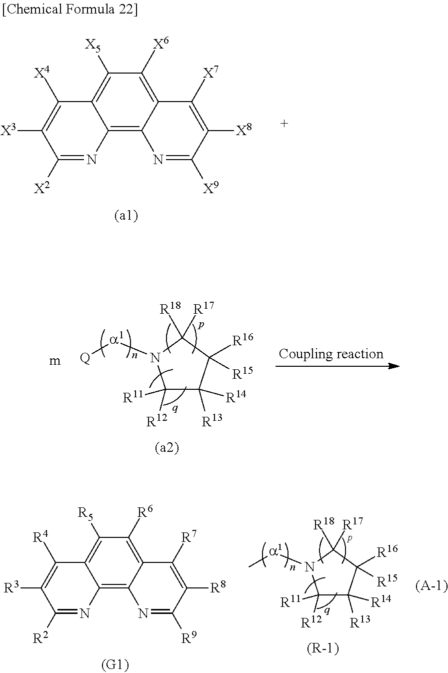

In the compound (a1), at least any one of X² to X⁹ represents halogen or a triflate group, and the others each independently represent hydrogen (including deuterium), an alkyl group having 1 to 10 carbon atoms, a cycloalkyl group having 3 to 10 carbon atoms, an alkoxy group having 1 to 6 carbon atoms, a cyclic secondary amino group having 2 to 10 carbon atoms, a substituted or unsubstituted aryl group having 6 to 30 carbon atoms, or a substituted or unsubstituted heteroaryl group having 1 to 30 carbon atoms.

In the compound (a2), Q represents a boronyl group (—B(OH)₂); c& represents a substituted or unsubstituted aryl-diyl group; n represents I or 2; in the case where n is 2; a plurality of substituted or unsubstituted aryl-diyl groups may be the same or different from each other; R¹¹ to R²⁶ each independently represent hydrogen (including deuterium) or an alkyl group having 1 to 10 carbon atoms; and p and q each independently represent 0 or 1. Any two of R¹¹ to R¹⁸ may be bonded to each other to form a ring. In the case where Q is a boronyl group in the compound (a2), a boronic ester, a cyclic-triolborate salt, or the like may be used.

In Synthesis Scheme (A-1), in is a positive number.

In General Formula (GI) above, at least any one of R² to R⁹ is a group represented by General Formula (R-1), and each of the other groups of R² to R⁰ independently represents hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, or a substituted or unsubstituted heteroaryl group having 1 to 30 carbon atoms. In General Formula (R-1), α¹ represents a substituted or unsubstituted arylene group having 6 to 30 carbon atoms; n represents 1 or 2; in the case where n is 2, a plurality of substituted or unsubstituted arylene groups having 6 to 30 carbon atoms may be the same or different from each other; R¹¹ to R²⁶ each independently represent hydrogen (including deuterium) or an alkyl group having 1 to 10 carbon atoms; and p and q each independently represent 0 or 1. Any two of R¹¹ to R¹⁸ may be bonded to each other to form a ring, and in the case where two or more of R² to R⁹ are groups represented by General Formula (R-1), the two or more of R² to R⁹ may be the same or different from each other.

Examples of a palladium catalyst that can be used in the coupling reaction represented by Synthesis Scheme (A-1) include palladium(II) acetate, tetrakis(triphenylphosphine)palladium(0), and bis(triphenylphosphine)palladium(II) dichloride. Examples of a ligand in the palladium catalyst include (±)-2,2′-bis(diphenylphosphino)-1,1′-binaphthyl, tri(ortho-tolyl)phosphine, triphenylphosphine, and tricyclohexylphosphine.

Examples of a base that can be used in the coupling reaction represented by Synthesis Scheme (A-1) include an organic base such as potassium tert-butoxide and an inorganic base such as potassium carbonate or sodium carbonate.

Examples of a solvent that can be used in the coupling reaction represented by the above synthesis scheme include toluene, xylene, mesitylene, benzene, tetrahydrofuran, and dioxane. However, the solvent that can be used is not limited to these solvents.

The reaction in the above synthesis scheme is not limited to the Suzuki-Miyaura reaction. A Buchwald-Hartwig reaction, a Migita-Kosugi-Stille coupling reaction using an organotin compound, a coupling reaction using a Grignard reagent, an Ullmann reaction using copper or a copper compound, a nucleophilic substitution reaction, or the like can be used.

A variety of kinds of the above compounds (a1) and (a2) are commercially available or can be synthesized.

The organic compound of one embodiment of the present invention can be synthesized in the above manner, but the present invention is not limited to this and other synthesis methods may be employed.

The structures described in this embodiment can be used in combination with any of the structures described in the other embodiments as appropriate.

### Embodiment 2

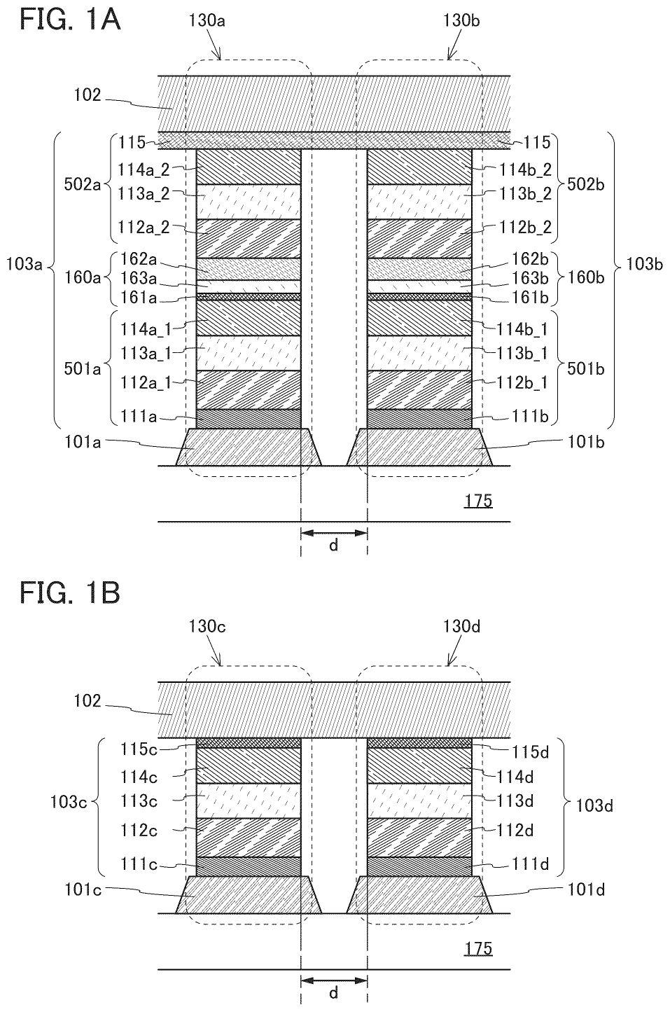

In this embodiment, a light-emitting device in which the organic compound of one embodiment of the present invention is used for an intermediate layer will be described with reference to FIGS. 1A and 1B.

FIG. 1A illustrates a schematic view of light-emitting devices 130a and 130b, which are tandem light-emitting devices formed over one insulating surface to be adjacent to each other. In each of the light-emitting devices 130a and 130b, part of an organic compound layer is formed through processing by a lithography method.

The light-emitting device 130a is located over an insulating layer 175 and includes a first electrode 101a that includes an anode, a second electrode 102 that includes a cathode, and an organic compound layer 103a. The organic compound layer 103a is located between the first electrode 101a and the second electrode 102. In the organic compound layer 103a, a first light-emitting unit 501a and a second light-emitting unit 502a are stacked with an intermediate layer 160a sandwiched therebetween. The first light-emitting unit 501a includes a first light-emitting layer 113a_1. The intermediate layer 160a includes a first layer 161a and a second layer 162a. The second light-emitting unit 502a includes a second light-emitting layer 113a_2 and an electron-injection layer 115. It can be said that the intermediate layer 160a is located between the first light-emitting layer I 13a_1 and the second light-emitting layer 113a_2.

In the organic compound layer 103a of the light-emitting device 130a, layers other than the electron-injection layer 115 are formed through processing by a lithography method. Thus, the layers other than the electron-injection layer 115 in the organic compound layer 103a are separate from those in the organic compound layer of the adjacent light-emitting device 130b. End portions (contours) of the layers other than the electron-injection layer 115 in the organic compound layer 103a are aligned or substantially aligned with each other in a direction perpendicular to a substrate. In other words, the first light-emitting layer 113a_1, the intermediate layer 160a (the first layer 161a and the second layer 162a), and the second light-emitting layer 113a_2 are separate from a first light-emitting layer 113b_1, an intermediate layer 160b (a first layer 161b and a second layer 162b), and a second light-emitting layer 113b_2. End portions (contours) of the first light-emitting layer 113a_1, the intermediate layer 160a (the first layer 161a and the second layer 162a), and the second light-emitting layer 113a_2 are aligned or substantially aligned with each other in a direction perpendicular to the substrate.

The light-emitting device 130b is located over the insulating layer 175 and includes a first electrode 101b that includes an anode, the second electrode 102 that includes the cathode, and an organic compound layer 103b. The organic compound layer 103b is located between the first electrode 101b and the second electrode 102. In the organic compound layer 103b, a first light-emitting unit 501b and a second light-emitting unit 502b are stacked with the internediate layer 160b sandwiched therebetween. The first light-emitting unit 501b includes the first light-emitting layer 113b_1. The intermediate layer 160b includes the first layer 161b and the second layer 162b. The second light-emitting unit 502b includes the second light-emitting layer 113b_2 and the electron-injection layer 115. It can be said that the intermediate layer 160b is located between the first light-emitting layer 113b_1 and the second light-emitting layer 113b_2.

In the organic compound layer 103b of the light-emitting device 130b, layers other than the electron-injection layer 115 are formed through processing by a lithography method. Thus, the layers other than the electron-injection layer 115 in the organic compound layer 103b are separate from those in the organic compound layer of the adjacent light-emitting device 130a. End portions (contours) of the layers other than the electron-injection layer 115 in the organic compound layer 103b are aligned or substantially aligned with each other in a direction perpendicular to the substrate. In other words, the first light-emitting layer 113b_1, the intermediate layer 160b (the first layer 161b and the second layer 162b), and the second light-emitting layer 113b_2 are separate from the first light-emitting layer 113a_1, the intermediate layer 160a (the first layer 161a and the second layer 162a), and the second light-emitting layer 113a_2. End portions (contours) of the first light-emitting layer 113b_1, the intermediate layer 160b (the first layer 161b and the second layer 162b), and the second light-emitting layer 113b_2 are aligned or substantially aligned with each other in a direction perpendicular to the substrate.

The electron-injection layer 115 and the second electrode 102 are preferably formed after the layers of the organic compound layer 103a other than the electron-injection layer 115 and the layers of the organic compound layer 103b other than the electron-injection layer 115 are formed through processing by a lithography method. In other words, the electron-injection layer 115 and the second electrode 102 are each preferably a continuous layer shared by the light-emitting devices 130a and 130b.

The electron-injection layer 115 is preferably formed using a donor substance (also referred to as an electron donor), in which case the driving voltages of the light-emitting devices can be reduced. Typical examples of the donor substance include alkali metals such as lithium (Li), which have a low work function, and compounds of the alkali metals.

In the case where processing by a lithography method is performed in a state where an electron-injection layer including such a donor substance serves as an interface of an organic compound layer, the influence of oxygen or water in the air and a chemical solution or water used during the process sometimes causes a light-emitting device to have a significantly increased driving voltage or greatly reduced current efficiency. However, in one embodiment of the present invention, since the electron-injection layer 115 is formed after the layers other than the electron-injection layer 115 are formed through processing by a lithography method, using a donor substance for the electron-injection layer 115 does not cause deterioration of the characteristics of the light-emitting devices.

In the case where the organic compound layers are formed through processing by a lithography method, a distance d of a gap between the layers of the organic compound layer 103a other than the electron-injection layer 115 and the layers of the organic compound layer 103b other than the electron-injection layer 115 can be shorter than the distance d in the case of employing mask vapor deposition, Specifically, the distance d can be reduced to less than 10 μm, less than or equal to 8 μm, less than or equal to 5 μm, less than or equal to 3 μm, less than or equal to 2 μm, less than or equal to 1.5 μm, less than or equal to 1 μm, or less than or equal to 0.5 μm. Using a light exposure apparatus for LSI can further shorten the distance d to less than or equal to 500 nm, less than or equal to 200 nm, less than or equal to 100 nm, or even less than or equal to 50 nm, for example, in a process over a Si wafer.

It is preferable that an insulating layer be provided in the gap between the layers of the organic compound layer 103a other than the electron-injection layer 115 and the layers of the organic compound layer 103b other than the electron-injection layer 115 to separate the layers of the organic compound layer 103a other than the electron-injection layer 115 from the layers of the organic compound layer 103b other than the electron-injection layer 115. In that case, there is a region where the insulating layer is in contact with the electron-injection layer 115 or the second electrode 102.

In the light-emitting device 130a, the first light-emitting unit 501a preferably includes a hole-injection layer 111a, a first hole-transport layer 112a_1, and a first electron-transport layer 114a_1 in addition to the first light-emitting layer l13a_1. The second light-emitting unit 502a preferably includes a second hole-transport layer 112a_2 and a second electron-transport layer 114a_2 in addition to the second light-emitting layer 113a_2 and the electron-injection layer 115. The intermediate layer 160a can include a third layer 163a between the first layer 161a and the second layer 162a. In the case where the surface of the light-emitting unit on the anode side is in contact with the intermediate layer 160a as in the second light-emitting unit 502a, the second layer 162a of the intermediate layer 160a, which is located on the cathode side, can also function as a hole-injection layer of the second light-emitting unit 502a, and thus, providing a hole-injection layer in such a light-emitting unit is optional.

In the light-emitting device 130b, the first light-emitting unit 501b preferably includes a hole-injection layer 111b, a first hole-transport layer 112b_1, and a first electron-transport layer 114b_1 in addition to the first light-emitting layer 113b_1. The second light-emitting unit 502b preferably includes a second hole-transport layer 112b_2 and a second electron-transport layer 114b_2 in addition to the second light-emitting layer 113b_2 and the electron-injection layer 115. The intermediate layer 160b can include a third layer 163b between the first layer 161b and the second layer 162b. In the case where the surface of the light-emitting unit on the anode side is in contact with the intermediate layer 160b as in the second light-emitting unit 502b, the second layer 162b of the intermediate layer 160b, which is located on the cathode side, can also function as a hole-injection layer of the second light-emitting unit 502b, and thus, providing a hole-injection layer in such a light-emitting unit is optional.

As illustrated in FIG. 1A, the uppermost one of the layers of the organic compound layer 103a other than the electron-injection layer 115 is preferably the second electron-transport layer 114a_2. Similarly, the uppermost one of the layers of the organic compound layer 103b other than the electron-injection layer 115 is preferably the second electron-transport layer 114b_2. The influence of oxygen or water in the air and a chemical solution or water used during the process can be smaller in the case where processing by a lithography method is performed at the interface on the second electron-transport layer 114a_2 and the second electron-transport layer 114b_2 than in the case where processing is performed at the interface on the second light-emitting layer 113a_2 and the second light-emitting layer 113b_2. Therefore, when the uppermost one of the layers of each organic compound layer other than the electron-injection layer 115 is the electron-transport layer, deterioration of the characteristics of the light-emitting devices due to the fabrication by a lithography method can be more easily avoided; processing by a lithography method is preferably performed at least above the second light-emitting layer 113a_2 and the second light-emitting layer 113b_2.

Although FIG. 1A illustrates an example in which each of the organic compound layers includes two light-emitting units, one embodiment of the present invention is not limited to this example. Each of the organic compound layers may include three or more light-emitting units. When a plurality of light-emitting units are stacked between a pair of electrodes with an intermediate layer sandwiched between the plurality of light-emitting units, the light-emitting device can perform high-luminance light emission with the current density kept low and can have high reliability. In addition, the light-emitting device can have low power consumption. Although not illustrated in FIG. 1A, each of the light-emitting units may include a hole-injection layer, a hole-transport layer, an electron-blocking layer, a hole-blocking layer, an electron-transport layer, an electron-injection layer, or the like in addition to the above-described components. Each layer may be a stack of two or more layers.

Note that in this specification, description referring to the structure of one of the light-emitting devices 130a and 130b can apply to the structure of the other of the light-emitting devices 130a and 130b.

The intermediate layer 160a sandwiched between the first light-emitting unit 501a and the second light-emitting unit 502a injects electrons into one of the first light-emitting unit 501a and the second light-emitting unit 502a and injects holes into the other of the first light-emitting unit 501a and the second light-emitting unit 502a when voltage is applied between the first electrode 101a and the second electrode 102, for example. When voltage is applied such that the potential of the second electrode 102 is higher than that of the first electrode 101a in FIG. 1A, for example, the intermediate layer 160a injects electrons into the first light-emitting unit 501a and injects holes into the second light-emitting unit 502a.

In the light-emitting device 130a illustrated as an example in FIG. 1A, when voltage is applied between a pair of electrodes (the first electrode 101a and the second electrode 102), electrons are injected from the cathode into the electron-injection layer 115 and holes are injected from the anode into the hole-injection layer 111a, so that current flows. Furthermore, electrons are injected from the first layer 161a of the intermediate layer 160a located on the anode side into the first electron-transport layer 114a_1 of the first light-emitting unit 501a, and holes are injected from the second layer 162a of the intermediate layer 160a located on the cathode side into the second hole-transport layer 112a_2 of the second light-emitting unit 502a. By recombination of the injected carriers (electrons and holes), excitons are formed. When carriers (electrons and holes) recombine and excitons are formed in the first light-emitting layer 113a_1 and the second light-emitting layer 113a_2 including light-emitting materials, the light-emitting materials included in the first light-emitting layer 113a_1 and the second light-emitting layer 113a_2 are brought into an excited state, causing light emission from the light-emitting materials.

It is preferable that the first layer 161a of the intermediate layer 160a located on the anode side be adjacent to the first electron-transport layer 114a_1 and be provided between the first electron-transport layer 114a_1 and the second light-emitting unit 502a as illustrated in FIG. 1A. With such a structure, electrons can be efficiently injected into the first light-emitting unit 501a.

For a lower driving voltage and more efficient light emission of the light-emitting device, a structure is preferably employed in which a barrier against electron injection from the intermediate layer 160a into the first electron-transport layer 114a_1 is lowered and electrons generated in the intermediate layer 160a are smoothly injected and transported into the first electron-transport layer 114a_1. In view of this, an alkali metal or an alkaline earth metal, which has a low work function, or a compound of an alkali metal or an alkaline earth metal is generally used for the intermediate layer 160a. However, the metal and the compound easily deteriorate by oxygen or water in the air and water or a chemical solution used during the processing step by a lithography method, causing a light-emitting device to have a significantly increased driving voltage or greatly reduced current efficiency.

In view of the above, it is preferable that the intermediate layer be formed using a material resistant to oxygen and water in the air and water and a chemical solution used during the processing step by a lithography method, and it is accordingly preferable that the intermediate layer 160a be formed using a metal that is stable with respect to oxygen and water in the air and resistant to water and a chemical solution. However, such a metal, which is stable and has a low electron-injection property, may form an electron injection barrier between the intermediate layer 160a and the first electron-transport layer 114a_1, causing the light-emitting device to have an increased driving voltage and reduced emission efficiency, for example.

In view of the above, in the light-emitting device of one embodiment of the present invention, the organic compound of one embodiment of the present invention is preferably used for the first layer 161a located on the anode side of the intermediate layer 160a, and the organic compound and the metal of one embodiment of the present invention are further preferably used. When the organic compound of one embodiment of the present invention and the metal are used, interaction between the organic compound of one embodiment of the present invention and the metal forms a donor level (a singly occupied molecular orbital (SOMO) level or a highest occupied molecular orbital (HOMO) level). This lowers a barrier against electron injection from the intermediate layer 160a into the first electron-transport layer 114a_1, and the electrons generated in the intermediate layer 160a can be injected and transported smoothly into the first electron-transport layer 114a_1. Accordingly, the intermediate layer can be formed to have a favorable electron-injection property and resistance to oxygen and water in the air and water and a chemical solution used during the processing step by a lithography method; thus, the light-emitting device can have a reduced driving voltage and high emission efficiency.

Since a metal with a low work function typified by an alkali metal and an alkaline earth metal and a compound of the metal with a low work function have high reactivity with oxygen and water, using the metal or the compound for a light-emitting device fabricated through processing by a lithography method may cause a reduction in emission efficiency, an increase in driving voltage, a reduction in driving lifetime, generation of shrinkage (a non-emission region at an end portion of a light-emitting portion), or the like, leading to deterioration in the characteristics or a reduction in the reliability of the light-emitting device. However, in one embodiment of the present invention, even when an alkali metal, an alkaline earth metal, or a compound of an alkali metal or an alkaline earth metal is used, a stabilized compound material is obtained through interaction between the alkali metal, the alkaline earth metal, or the compound and the organic compound of one embodiment of the present invention, so that an intermediate layer having resistance to oxygen and water in the air and water and a chemical solution used during the processing step by a lithography method can be formed. An alkali metal, an alkaline earth metal, or a compound of an alkali metal or an alkaline earth metal is preferably used as the metal in one embodiment of the present invention, in which case the donor level (SOMO level or HOMO level) that is formed by interaction between the alkali metal, the alkaline earth metal, or the compound and the organic compound of one embodiment of the present invention can be a high energy level, a barrier against electron injection from the intermediate layer 160a into the first electron-transport layer 114a_1 can be lowered, and the electrons generated in the intermediate layer 160a can be injected and transported smoothly into the first electron-transport layer 114a_1.

As the metal in the light-emitting device of one embodiment of the present invention, it is possible to use any one of transition metals (metal elements belonging to Group 3 to Group 11) and main-group metal elements belonging to Group 12 to Group 14. These metals have low reactivity with oxygen and water in the air and water and a chemical solution used in a lithography process. Thus, using any of these metals in the light-emitting device is advantageous in that the metals hardly cause deterioration due to water and oxygen, which would be a matter of concern in the case of using a metal with a low work function. On the other hand, transition metals (metal elements belonging to Group 3 to Group 11) and main-group metal elements belonging to Group 12 to Group 14, which are stable and have a low electron-injection property, cause the light-emitting device to have reduced emission efficiency, an increased driving voltage, and a reduced driving lifetime, for example. However, in one embodiment of the present invention, even when any one of transition metals (metal elements belonging to Group 3 to Group 11) and main-group metal elements belonging to Group 12 to Group 14 is used, a donor level (SOMO level or HOMO level) can be formed by interaction between the metal and the organic compound of one embodiment of the present invention; this allows a barrier against electron injection from the intermediate layer 160a into the first electron-transport layer 114a_1 to be lowered and enables the electrons generated in the intermediate layer 160a to be injected and transported smoothly into the first electron-transport layer 114a_1. The above structure is preferably employed, in which case an intermediate layer that has resistance to oxygen and water in the air and water and a chemical solution used during the processing step by a lithography method can be formed. Thus, one embodiment of the present invention can provide a light-emitting device having high moisture resistance, high water resistance, high oxygen resistance, high chemical resistance, a low driving voltage, and high emission efficiency.

In the interaction between the metal and the organic compound of one embodiment of the present invention, the sum of the number of electrons of the organic compound of one embodiment of the present invention and the number of electrons of the metal is preferably an odd number, in which case the stabilization energy is lower and a donor level (SOMO level or HOMO level) can be formed as a high energy level. Accordingly, in the case where the number of electrons of the organic compound of one embodiment of the present invention is an even number, the metal preferably belongs to an odd-numbered group in the periodic table.

In a general fabrication process of a light-emitting device, an EL layer, particularly an intermediate layer, of the light-emitting device is formed by a vacuum evaporation method in many cases. In those cases, it is preferable to use a material that can be easily deposited by vacuum evaporation, i.e., a material with a low melting point. The metals belonging to Group 11 and Group 13 have low melting points and thus, they can be suitably used for vacuum evaporation. The metals belonging to Group 11 and Group 13 are preferable because they are stable with respect to oxygen and water in the air. A vacuum evaporation method is preferably used, in which case a metal atom and an organic compound can be easily mixed.

Furthermore, Ag and In can be used also as a cathode material. The intermediate layer and the cathode are preferably formed using the same material to facilitate the fabrication of the light-emitting device and to reduce the manufacturing cost thereof.

Next, details of the structure of the intermediate layer that can be used for the light-emitting devices 130a and 130b are described.

### <<First Layer>>

The organic compound of one embodiment of the present invention is preferably used for the first layers (the first layer 161a and the first layer 161b) located on the anode side of the intermediate layers, and a mixed layer or a stacked-layer structure including the metal and the organic compound of one embodiment of the present invention is further preferably used for each of the first layers. The metal and the organic compound of one embodiment of the present invention are a combination in which a SOMO or a HOMO that can function as a donor level can be formed by interaction between the metal and the organic compound of one embodiment of the present invention. When a layer that includes this combination is used as each of the first layers of the intermediate layers, electrons generated in the first layers can be easily injected into the first light-emitting units. Alternatively, electrons generated in the second layers (the second layer 162a and the second layer 162b) of the intermediate layers, which are located on the cathode side, can be easily injected into the first light-emitting units. This facilitation of electron injection into the first light-emitting units enables the light-emitting devices to have a reduced driving voltage and increased emission efficiency.

For each of the first layers located on the anode side of the intermediate layers, the organic compound of one embodiment of the present invention is preferably and the mixed layer or the stacked-layer structure including the metal and the organic compound of one embodiment of the present invention is further preferably used. In the case of the stacked-layer structure, it is preferable that the organic compounds of one embodiment of the present invention be stacked on the anode side and the metals be stacked on the cathode side. When the mixed layer or the stacked-layer structure of the metal and the organic compound of one embodiment of the present invention is used, interaction between these substances is facilitated, whereby a barrier against electron injection from the second light-emitting units into the first light-emitting units can be further lowered owing to the metal and the organic compound of one embodiment of the present invention functioning as electron donors. This facilitates electron injection into the first light-emitting unit and accordingly enables the light-emitting device to have a further reduced driving voltage and further increased emission efficiency. The mixed layer of the metal and the organic compound of one embodiment of the present invention is less likely to be crystallized than when having a stacked-layer structure of the metals and the organic compounds of one embodiment of the present invention. Accordingly, the first layer of the intermediate layer is not easily crystallized even when affected by oxygen or water in the air and a chemical solution or water during processing by a lithography method for forming part of the organic compound layer. An increase in driving voltage or a reduction in current efficiency of the light-emitting device due to crystallization of the intermediate layer can be prevented. Thus, the mixed layer can be used more suitably for the intermediate layer of the light-emitting device in which part of the organic compound layer is formed through processing by a lithography method than in the case where the stacked-layer structure is employed.

### <Metal>

As the metal, a main-group metal or a transition metal can be used.

As the main-group metal, an alkali metal (Group 1 element) such as Li, Na, K, or Cs, an alkaline earth metal (Group 2 element) such as Mg, Ca, or Ba, a Group 12 element such as Zn, an earth metal (Group 13 element) such as Al or In, a Group 14 element such as Sn, or a compound of a Group 1, 2, 13, or 14 element can be used.

An alkali metal, an alkaline earth metal, or a compound of an alkali metal or an alkaline earth metal is preferably used as the metal, in which case the donor level formed by interaction between the alkali metal, the alkaline earth metal, or the compound and the organic compound of one embodiment of the present invention can be a high energy level; accordingly, electrons generated in the intermediate layer can be smoothly injected and transported into the electron-transport layer, enabling the light-emitting device to have a low driving voltage and emit light with high efficiency.

As the transition metal, any of Group 3 elements, including Y and lanthanoids such as Eu and Yb, Group 7 elements such as Mn, Group 8 elements such as Fe, Group 9 elements such as Co, Group 10 elements such as Ni and Pt, Group 11 elements such as Cu, Ag, and Au, and a compound of a Group 3, 7, 8, 9, 10, or 11 element can be used. The transition metal is preferable because it has low reactivity with components of the air such as water and oxygen.

It is further preferable to use a metal belonging to an odd-numbered group (Group 1, 3, 5, 7, 9, 11, or 13) among the above metals. Among transition metals belonging to the odd-numbered groups, a metal having one electron (unpaired electron) in an orbital of the outermost shell is particularly preferable because a combination of this metal and the organic compound of one embodiment of the present invention easily forms a SOMO level.

A metal that has a low melting point and that can be deposited by a vacuum evaporation method is preferably used because a mixed layer of this metal and an organic compound is easy to form. Specifically, for example, the metals belonging to Group 11 and Group 13 have low melting points and thus, they can be suitably used for vacuum evaporation. The metals belonging to Group 11 and Group 13 are preferable because they are stable with respect to oxygen and water in the air.

Note that an organic compound with an electron-transport property may be further added to the first layer of the intermediate layer, in addition to the metal and the organic compound of one embodiment of the present invention. The organic compound with an electron-transport property is preferably a substance having an electron mobility higher than or equal to 1×10⁻⁷ cm²/Vs, further preferably higher than or equal to 1×10⁻⁶ cm²/Vs, when the square root of electric field strength [V/cm] is 600. Note that any other substance can be used as long as the substance has an electron-transport property higher than a hole-transport property.

An organic compound having a π-electron deficient heteroaromatic ring is preferable as the organic compound with an electron-transport property. The organic compound having a π-electron deficient heteroaromatic ring is preferably one or more of an organic compound having a heteroaromatic ring with an azole skeleton, an organic compound having a heteroaromatic ring with a pyridine skeleton, an organic compound having a heteroaromatic ring with a diazine skeleton, and an organic compound having a heteroaromatic ring with a triazine skeleton.

Specific examples of the organic compound with an electron-transport property include the following compounds: organic compounds having an azole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′, 2″-(1,3,5-benzenetriyl)tris(1-phenyl-1H-benzimidazole) (abbreviation: TP3I), 2-[3-(dibenzothiophen-4-yl)phenyl]-1-phenyl-1H-benzimidazole (abbreviation: mDBTBIm-11), and 4,4′-bis(5-methylbenzoxazol-2-yl)stilbene (abbreviation: BzOs); organic compounds having a heteroaromatic ring with a pyridine skeleton, such as 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), bathophenanthroline (abbreviation: BPhen), bathocuproine (abbreviation: BCP), 2,9-di(naphthalen-2-yl)-4,7-diphenyl-1,10-phenanthroline (abbreviation: NBPhen), and 2,2′-(1,3-phenylene)bis(9-phenyl-1,10-phenanthroline) (abbreviation: mPPhen2P); organic compounds having a diazine skeleton, such as 2-[3-(dibenzothiophen-4-yl)phenyl]dibenzo[f,h]quinoxaline (abbreviation: 2mDBTPDBq-1I), 2-[3′-(dibenzothiophen-4-yl)biphenyl-3-yl]dibenzo[f,h]quinoxaline (abbreviation: 2mDBTBPDBq-1I), 2-[3′-(9H-carbazol-9-yl)biphenyl-3-yl]dibenzo[f,h]quinoxaline (abbreviation: 2mCzBPDBq), 2-[4′-(9-phenyl-9H-carbazol-3-yl)-3,1′-biphenyl-1-yl]dibenzo[f,h]quinoxaline (abbreviation: 2mpPCBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[fh]quinoxaline (abbreviation: 6mDBTPDBq-II), 9-[3′-(dibenzothiophen-4-yl)biphenyl-3-yl]naphtho[1′, 2′: 4,5]furo[2,3-b]pyrazine (abbreviation: 9mDBt3PNfpr), 9-[3′-(dibenzothiophen-4-yl)biphenyl-4-yl]naphtho[1′, 2′: 4,5]furo[2,3-b]pyrazine (abbreviation: 9pmDBtBPNfpr), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 9,9′-[pyrimidine-4,6-diylbis(biphenyl-3,3′-diyl)]bis(9H-carbazole) (abbreviation: 4,6mCzBP2Pm), 8-(biphenyl-4-yl)-4-[3-(dibenzothiophen-4-yl)phenyl]-[1]benzofuro[3,2-d]pyrimidine (abbreviation: 8BP-4mDBtPBfpm), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpn), 8-[3′-(dibenzothiophen-4-yl)biphenyl-3-yl]naphtho[1′, 2′: 4,5]furo[3,2-d]pyrinidine (abbreviation: 8mDBtBPNfpm), 8-[(2,2′-binaphthalen)-6-yl]-4-[3-(dibenzothiophen-4-yl)phenyl]-[1]benzofuro[3,2-d]pyrimidine (abbreviation: 8(PN2)-4mDBtPBfpm), 6-(biphenyl-3-yl)-4-[3,5-bis(9H-carbazol-9-yl)phenyl]-2-phenylpyrimidine (abbreviation: 6mBP-4Cz2PPm), 2,6-bis(4-naphthalen-1-ylphenyl)-4-[4-(3-pyridyl)phenyl]pyrimidine (abbreviation: 2,4NP-6PyPPm), 4-[3,5-bis(9H-carbazol-9-yl)phenyl]-2-phenyl-6-(biphenyl-4-yl)pyrimidine (abbreviation: 6BP-4Cz2PPm), and 7-[4-(9-phenyl-9H-carbazol-2-yl)quinazolin-2-yl]-7H-dibenzo[c,g]carbazole (abbreviation: PC-cgDBCzQz); and organic compounds having a triazine skeleton, such as 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 2-[3′-(9,9-dimethyl-9H-fluoren-2-yl)biphenyl-3-yl]-4,6-diphenyl-1,3,5-triazine (abbreviation: mFBPTzn), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 2,4,6-tris[3′-(pyridin-3-yl)biphenyl-3-yl]-1,3,5-triazine (abbreviation: TmPPPyTz), 2-[3-(2,6-dimnethyl-3-pyridinyl)-5-(9-phenanthrenyl)phenyl]-4,6-diphenyl-1,3,5-triazine (abbreviation: mPn-mDMePyPTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), 2-[3′-(triphenylen-2-yl)biphenyl-3-yl]-4,6-diphenyl-1,3,5-triazine (abbreviation: mTpBPTzn), 3-[9-(4,6-diphenyl-1,3,5-triazin-2-yl)-2-dibenzofuiranyl]-9-phenyl-9H-carbazole (abbreviation: PCDBfTzn), and 2-(biphenyl-3-yl)-4-phenyl-6-{8-[(1,1′: 4′, 1″-terphenyl)-4-yl]-1-dibenzofuranyl}-1,3,5-triazine (abbreviation: mBP-TPDBfTzn).

Among the above organic compounds, the organic compound having a phenanthroline ring such as BPhen, BCP, NBPhen, or mPPhen2P is preferable, and an organic compound having a phenanthroline ring dimer structure, such as mPPhen2P, is further preferable because of its high stability.

The organic compound with an electron-transport property preferably has 25 to 100 carbon atoms. When having 25 to 100 carbon atoms, the organic compound can have excellent sublimability, and thus, thernal decomposition of the organic compound during vacuum evaporation can be inhibited and the efficiency of use of the material can be high. An organic compound having T_{g} higher than or equal to 100° C. can be used as the organic compound. In that case, the intermediate layer is not easily crystallized. Accordingly, the intermediate layer is not easily crystallized even when affected by oxygen or water in the air and a chemical solution or water during processing by a lithography method for forming part of the organic compound layer or subjected to a heating step for desorbing water adsorbed onto the organic compound layer. An increase in driving voltage or a reduction in current efficiency of the light-emitting device due to crystallization of the intermediate layer can be accordingly prevented. Thus, when the organic compound having T_{g} higher than or equal to 100° C. is used as the organic compound with an electron-transport property, the organic compound with an electron-transport property can be suitably used for the intermediate layer of the light-emitting device in which part of the organic compound layer is formed through processing by a lithography method.

Examples of an organic compound having a phenanthroline ring and T_{g} higher than or equal to 100° C. include NBPhen (T_{g}: 165° C.), mPPhen2P (T_{g}: 135° C.), 2,2′-(biphenyl-4,4′-diyl)bis(9-phenyl-1,10-phenanthroline) (abbreviation: PPhen2BP) (T_{g}: 166° C.), 2,2′-biphenyl-3,3′-diylbis(9-phenyl-1,10-phenanthroline) (abbreviation: mPPhen2BP) (T_{g}: 144° C.), 2,8-bis(phenanthrolin-5-yl)dibenzofuran (abbreviation: 2,8Phen2DBf) (T_{g}: 210° C.), and 5,5′, 5″-(benzene-1,3,5-triyl)tri-1,10-phenanthroline (abbreviation: Phen3P) (T_{g}: 257° C.).

As the organic compound with an electron-transport property, an organic compound with an acid dissociation constant pKₐ higher than or equal to 4 and lower than 8 can be used. The organic compound with an electron-transport property preferably has such an acid dissociation constant to have a poor hole-transport property, in which case the hole-transport property in the first layer 161a of the intermediate layer 160a can be reduced and hole transport from the first layer 161a to the second layer 162a can be prevented, enabling the light-emitting device to have high efficiency. An excessively high acid dissociation constant pKₐ leads to high solubility in water and thus causes low resistance to water and a chemical solution used in the process by a lithography method. Thus, the acid dissociation constant pKₐ of the organic compound with an electron-transport property is preferably higher than or equal to 4 and lower than 8.

The molar ratio of the metal to the organic compound of one embodiment of the present invention (the sum of the organic compound of one embodiment of the present invention and the organic compound with an electron-transport property in the case where the organic compound with an electron-transport property is further added) is preferably greater than or equal to 0.1 and less than or equal to 10, further preferably greater than or equal to 0.2 and less than or equal to 5, still further preferably greater than or equal to 0.5 and less than or equal to 2. Alternatively, the volume ratio of the metal to the organic compound of one embodiment of the present invention is preferably greater than or equal to 0.01 and less than or equal to 0.3, further preferably greater than or equal to 0.02 and less than or equal to 0.2, still further preferably greater than or equal to 0.05 and less than or equal to 0.1. Mixing the metal and the organic compound of one embodiment of the present invention in such a ratio enables providing the intermediate layer having a favorable electron-injection property. In the case where the organic compound with an electron-transport property is further added, the volume ratio of the organic compound of one embodiment of the present invention to the organic compound with an electron-transport property is preferably greater than or equal to 0.1 and less than or equal to 10, further preferably greater than or equal to 0.2 and less than or equal to 5, still further preferably greater than or equal to 0.5 and less than or equal to 2. Mixing the organic compound of one embodiment of the present invention and the organic compound with an electron-transport property in such a ratio enables providing the intermediate layer having a favorable electron-transport property.

The thickness of the first layer 161a of the intermediate layer 160a, which is located on the anode side, is preferably greater than or equal to 3 nm and less than or equal to 20 nm, further preferably greater than or equal to 5 nm and less than or equal to 10 nm. In that case, the composite material in which the metal and the organic compound of one embodiment of the present invention are mixed can favorably function, enabling high emission efficiency of the light-emitting device.

### <<Second Layer>>

In the case where the mixed layer or the stacked-layer structure that includes the metal and the organic compound of one embodiment of the present invention is used for the first layer of the intermediate layer, a layer that includes a third organic compound and a fourth organic compound (which will be described later in detail) is preferably used for the second layer to enable favorable injection of holes into the light-emitting layer on the upper side.

### <Third Organic Compound>

As the third organic compound, an organic compound with a hole-transport property is preferably used. As the organic compound with a hole-transport property, any of a variety of organic compounds such as aromatic amine compounds, heteroaromatic compounds, aromatic hydrocarbons, and high molecular compounds (e.g., oligomers, dendrimers, and polymers) can be used. Note that the organic compound with a hole-transport property preferably has a hole mobility higher than or equal to 1×10⁻⁶ cm²/Vs. The organic compound with a hole-transport property is preferably a compound having a condensed aromatic hydrocarbon ring or a π-electron rich heteroaromatic ring. As the condensed aromatic hydrocarbon ring, an anthracene ring, a naphthalene ring, or the like is preferable. As the π-electron rich heteroaromatic ring, a condensed aromatic ring having at least one of a pyrrole skeleton, a furan skeleton, and a thiophene skeleton is preferable; specifically, a carbazole ring, a dibenzothiophene ring, or a ring in which an aromatic ring or a heteroaromatic ring is further fused to the carbazole ring or the dibenzothiophene ring is preferable.

Such an organic compound with a hole-transport property further preferably has any of a carbazole skeleton, a dibenzofuran skeleton, a dibenzothiophene skeleton, and an anthracene skeleton. In particular, an aromatic amine having a substituent with a dibenzofuran ring or a dibenzothiophene ring, an aromatic monoamine having a naphthalene ring, or an aromatic monoamine in which a 9-fluorenyl group is bonded to the nitrogen of the amine through an arylene group may be used. Note that the organic compound with a hole-transport property preferably has an N,N-bis(4-biphenyl)amino group to enable fabrication of a light-emitting device having a long lifetime.

Specific examples of the organic compound with a hole-transport property include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBBIBP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′, 4″-diphenyltriphenylamine (abbreviation: BBAβNB), 4-[4-(2-naphthyl)phenyl]-4′, 4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4′ phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiApNBi), 4-phenyl-4′-(1-naphthyl)triphenylarnine (abbreviation: αNBA1BP), 4,4′-bis(1-naphthyl)triphenylamine (abbreviation: αNBBIBP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBiIBP-02), 4-[4′-(carbazol-9-yl)biphenyl-4-yl]-4′-(2-naphthyl)-4″-phenyltriphenylamine (abbreviation: YGTBiβNB), NV-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBAIBP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(biphenyl-4-yl)-NV-[4-(9-phenyl-9H-carbazol-3-yl)phenyl]-9,9-dimethyl-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

As the organic compound with a hole-transport property, any of the following aromatic amine compounds can also be used: N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), and 1,3,5-tris[N-(4-diphenylaminophenyl)-N-phenylamino]benzene (abbreviation: DPA3B).

### <Fourth Organic Compound>

As the fourth organic compound, a substance having an acceptor property with respect to the third organic compound is preferably used. As the substance with an acceptor property, an organic compound having an electron-withdrawing group (e.g., a halogen group or a cyano group) is preferably used, and an organic compound which has one or more halogen groups, one or more cyano groups, or both one or more halogen groups and one or more cyano groups and in which the total number of the one or more halogen groups, the one or more cyano groups, or both the one or more halogen groups and the one or more cyano groups is four or more is further preferably used. Specific examples include 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F₆-TCNNQ), and 2-(7-dicyanomethylen-1,3,4,5,6,8,9,10-octafluoro-7H11-pyren-2-ylidene)malononitrile. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group, a halogen group such as a fluoro group, or the like) has a very high electron-accepting property and thus is preferable. Specific examples include α,α′,α″-,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance with an acceptor property, a transition metal oxide such as molybdenum oxide, vanadium oxide, ruthenium oxide, tungsten oxide, or manganese oxide can be used, other than the above-described organic compounds.

A signal is preferably observed by electron spin resonance in the second layer. For example, the density of spins attributed to a signal observed at a g-factor of approximately 2.00 is preferably higher than or equal to 1×10¹⁷ spins/cm³, further preferably higher than or equal to 1×10¹⁸ spins/cr³, still further preferably higher than or equal to 1×10¹⁹ spins/cm³. In that case, the second layer can function as a charge-generation layer. Furthermore, the light-emitting device can have a low driving voltage and high efficiency.

### <<Third Layer>>

Between the first layer and the second layer of the intermediate layer, the third layer for enabling smooth electron transfer between the two layers may be provided.

The third layer includes a substance with an electron-transport property and has a function of preventing interaction between the first layer and the second layer and transferring electrons smoothly. The LUMO level of the substance with an electron-transport property included in the third layer is preferably between the LUMO level of the acceptor substance in the second layer and the LUMO level of the organic compound included in a layer (the first electron-transport layer 114a_1 in the first light-emitting unit 501a and the first electron-transport layer 114b_1 in the first light-emitting unit 501b in FIG. 1A) which is included in the light-emitting unit on the first electrode side and is in contact with the intermediate layer. As a specific value of the energy level, the LUMO level of the substance with an electron-transport property in the third layer is preferably higher than or equal to −5.0 eV, further preferably higher than or equal to −5.0 eV and lower than or equal to −3.0 eV, still further preferably higher than or equal to −4.30 eV and lower than or equal to −3.00 eV, yet still further preferably higher than or equal to −4.30 eV and lower than or equal to −3.30 eV, in which case electrons generated in the second layer can be easily injected into the first layer and accordingly an increase in the driving voltage of the light-emitting device can be inhibited. Note that as the substance with an electron-transport property in the third layer, a phthalocyanine-based material or a metal complex having a metal-oxygen bond and an aromatic ligand is preferably used.

Specifically, it is possible to use a perylenetetracarboxylic acid derivative such as diquinoxalino[2,3-a:2′, 3′-c]phenazine (abbreviation: HATNA), 2,3,8,9,14,15-hexafluorodiquinoxalino[2,3-a:2′, 3′-c]phenazine (abbreviation: HATNA-F6), 3,4,9,10-perylenetetracarboxylic diimide (abbreviation: PTCDI), or 3,4,9,10-perylenetetracarboxyl-bis-benzimidazole (abbreviation: PTCBI), (C₆₀—Iₕ)[5,6]fullerene (abbreviation: C₆₀), (C₇₀-D₅ₕ)[5,6]fullerene (abbreviation: C₇₀), or phthalocyanine (abbreviation: H₂Pc). Alternatively, it is possible to use a metal phthalocyanine containing copper, zinc, cobalt, iron, chromium, nickel, or the like or a derivative thereof, such as copper phthalocyanine (abbreviation: CuPc), zinc phthalocyanine (abbreviation: ZnPc), cobalt phthalocyanine (abbreviation: CoPc), iron phthalocyanine (abbreviation: FePe), tin phthalocyanine (abbreviation: SnPc), tin oxide phthalocyanine (abbreviation: SnOPc), titanium oxide phthalocyanine (abbreviation: TiOPc), or vanadium oxide phthalocyanine (abbreviation: VOPc). It is particularly preferable to use a phthalocyanine-based metal complex such as copper phthalocyanine or zinc phthalocyanine or 2,3,8,9,14,15-hexafluorodiquinoxalino[2,3-a:2′, 3′-c]phenazine.

The thickness of the third layer is preferably greater than or equal to I nm and less than or equal to 10 nm, further preferably greater than or equal to 2 nm and less than or equal to 5 nm.

Although an example where the organic compound of one embodiment of the present invention is used for the intermediate layer of the light-emitting device in which part of the organic compound layer is formed through processing by a lithography method is described in this embodiment, one embodiment of the present invention is not limited thereto. The organic compound of one embodiment of the present invention may be used for an intermediate layer of a light-emitting device in which an organic compound layer is not formed through processing by a lithography method.

FIG. 1B illustrates a schematic view of light-emitting devices 130c and 130d, which are single-type light-emitting devices formed over one insulating surface to be adjacent to each other. In each of the light-emitting devices 130c and 130d, all of the organic compound layers are formed through processing by a lithography method.

The light-emitting device 130c is located over the insulating layer 175 and includes a first electrode 101c that includes an anode, the second electrode 102 that includes a cathode, and an organic compound layer 103c. The organic compound layer 103c is located between the first electrode 101c and the second electrode 102. The organic compound layer 103c includes a light-emitting layer 113c and an electron-injection layer 115c.

In the light-emitting device 130c, the organic compound layer 103c is formed through processing by a lithography method. Thus, the organic compound layer 103c is separate from the organic compound layer of the adjacent light-emitting device 130d. End portions (contours) of the layers of the organic compound layer 103c are aligned or substantially aligned with each other in a direction perpendicular to a substrate. That is, the light-emitting layer 113c and the electron-injection layer 115c are separate from a light-emitting layer 113d and an electron-injection layer 115d. End portions (contours) of the light-emitting layer 113c and the electron-injection layer 115c are aligned or substantially aligned with each other in a direction perpendicular to a substrate.

The light-emitting device 130d is located over the insulating layer 175 and includes a first electrode 101d that includes an anode, the second electrode 102 that includes a cathode, and an organic compound layer 103d. The organic compound layer 103d is located between the first electrode 101d and the second electrode 102. The organic compound layer 103d includes the light-emitting layer 113d and the electron-injection layer 115d.

In the light-emitting device 130d, the organic compound layer 103d is formed through processing by a lithography method. Thus, the organic compound layer 103d is separate from the organic compound layer of the adjacent light-emitting device 130c. End portions (contours) of the layers of the organic compound layer 103d are aligned or substantially aligned with each other in a direction perpendicular to a substrate. That is, the light-emitting layer 113d and the electron-injection layer 115d are separate from the light-emitting layer 113c and the electron-injection layer 115c. End portions (contours) of the light-emitting layer 113d and the electron-injection layer 115d are aligned or substantially aligned with each other in a direction perpendicular to a substrate.

The second electrode 102 is preferably formed after all the layers of the organic compound layers 103c and 103d are formed through processing by a lithography method. In other words, the second electrode 102 is preferably a continuous layer shared by the light-emitting devices 130c and 130d.

In the case where the organic compound layers are formed through processing by a lithography method, the distance d of the gap between the layers of the organic compound layer 103c and the layers of the organic compound layer 103d can be shorter than the distance d in the case of employing mask vapor deposition. Specifically, the distance d can be reduced to less than 10 μm, less than or equal to 8 μm, less than or equal to 5 μm, less than or equal to 3 μm, less than or equal to 2 μm, less than or equal to 1.5 μm, less than or equal to 1 μm, or less than or equal to 0.5 μm. Using a light exposure apparatus for LSI can further shorten the distance d to less than or equal to 500 nm, less than or equal to 200 nm, less than or equal to 100 nm, or even less than or equal to 50 nm, for example, in a process over a Si wafer.

It is preferable that an insulating layer be provided in the gap between the layers of the organic compound layer 103c and the layers of the organic compound layer 103d and the layers of the organic compound layer 103c are separated from the respective layers of the organic compound layer 103d. In that case, there is a region where the insulating layer is in contact with the second electrode 102.

In the light-emitting device 130c, the organic compound layer 103c preferably includes a hole-injection layer 111c, a hole-transport layer 112c, and an electron-transport layer 114c in addition to the light-emitting layer 113c and the electron-injection layer 115c. In the light-emitting device 130d, the organic compound layer 103d preferably includes a hole-injection layer 111d, a hole-transport layer 112d, and an electron-transport layer 114d in addition to the light-emitting layer 113d and the electron-injection layer 115d. Each of the organic compound layers may include a hole-injection layer, a hole-transport layer, an electron-blocking layer, a hole-blocking layer, an electron-transport layer, an electron-injection layer, or the like in addition to the above-described components. Each layer may be a stack of two or more layers.

For the manufacturing method of each of the light-emitting devices 130c and 130d, processing by a lithography method is performed after all the layers of the organic compound layers are formed, and then the second electrode 102 is formed. As described above, in the case where processing by a lithography method is performed in a state where the electron-injection layer including a donor substance serves as an interface of the organic compound layer, the influence of oxygen or water in the air and a chemical solution or water used during the process sometimes causes a light-emitting device to have a significantly increased driving voltage or greatly reduced current efficiency. Thus, for each of the electron-injection layers 115c and 115d, the organic compound of one embodiment of the present invention is preferably used and the mixed layer or the stacked-layer structure including the metal and the organic compound of one embodiment of the present invention is further preferably used. Specifically, the above-described structure of the first layer of the intermediate layer that can be used for each of the light-emitting devices 130a and 130b is preferably employed for the electron-injection layer 115c and the electron-injection layer 115d.

Using the organic compound of one embodiment of the present invention or using the mixed layer or the stacked-layer structure including the metal and the organic compound of one embodiment of the present invention for each of the electron-injection layers 115c and 115d enables the light-emitting devices to have favorable characteristics even when processing by a lithography method is performed in a state where the electron-injection layers 115c and 115d each serve as an interface of the organic compound layer.

The structures described in this embodiment can be used in combination with any of the structures described in the other embodiments as appropriate.

### Embodiment 3

In this embodiment, structures other than the intermediate layer, which includes the organic compound of one embodiment of the present invention, of the light-emitting device are described.

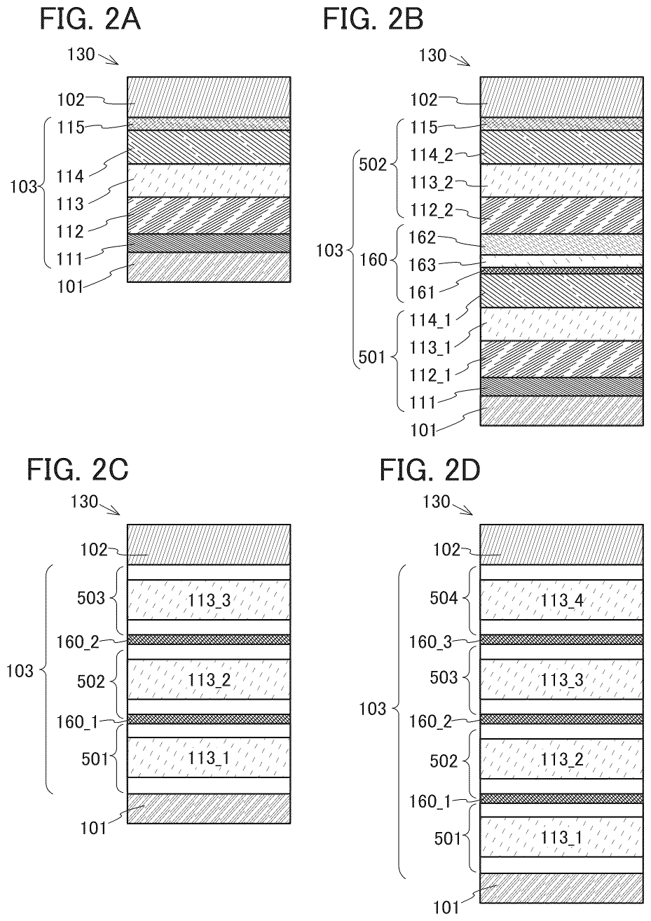

FIG. 2A illustrates a light-emitting device 130. The light-emitting device 130 includes an organic compound layer 103 that includes a light-emitting layer 113, between a first electrode 101 that includes an anode and the second electrode 102 that includes a cathode.

FIG. 2B illustrates the light-emitting device 130 that is another example of the light-emitting device of one embodiment of the present invention. The light-emitting device 130 is a tandem light-emitting device. The light-emitting device 130 includes a first light-emitting unit 501 including a first light-emitting layer 113_1, a second light-emitting unit 502 including a second light-emitting layer 113_2, and an intermediate layer 160, as the organic compound layer 103.

Although a light-emitting device that includes one intermediate layer 160 and two light-emitting units is described as an example in this embodiment, a light-emitting device that includes n intermediate layer(s) (n is an integer greater than or equal to 1) and n+1 light-emitting units may be employed.

For example, the light-emitting device 130 illustrated in FIG. 2C is an example of the tandem light-emitting device in which n is 2 and which includes the first light-emitting unit 501, a first intermediate layer 160_1, the second light-emitting unit 502, a second intermediate layer 160_2, and a third light-emitting unit 503, as the organic compound layer 103. The color gamut of light emitted by a light-emitting layer in one light-emitting unit may be the same as or different from that of light emitted by a light-emitting layer in another light-emitting unit. In addition, the light-emitting layer may have a single-layer structure or a stacked-layer structure. For example, the light-emitting layers in the first and third light-emitting units emit light in the blue range while the stacked light-emitting layers in the second light-emitting unit emit light in the red range and light in the green range, whereby white light emission can be obtained.

The light-emitting device 130 illustrated in FIG. 2D is an example of the tandem light-emitting device in which n is 3 and which includes the first light-emitting unit 501, the first intermediate layer 160_1, the second light-emitting unit 502, the second intermediate layer 1602, the third light-emitting unit 503, a third intermediate layer 160_3, and a fourth light-emitting unit 504, as the organic compound layer 103. The first light-emitting unit 501 includes the first light-emitting layer 113_1, the second light-emitting unit 502 includes the second light-emitting layer 113_2, the third light-emitting unit 503 includes a third light-emitting layer 113_3, and the fourth light-emitting unit 504 includes a fourth light-emitting layer 113_4. The color gamut of light emitted by a light-emitting layer in one light-emitting unit may be the same as or different from that of light emitted by a light-emitting layer in another light-emitting unit. In addition, the light-emitting layer may have a single-layer structure or a stacked-layer structure. For example, any three of the four light-emitting units can be blue (B), and the other one can be green (G); any two of the four light-emitting units can be blue (B), and the other two can be yellow (Y); alternatively, any one of the four light-emitting units can be red (R), another one can be green (G), and the other two can be blue (B).

The light-emitting device 130 may be fabricated using a lithography method, for example. In the case of the light-emitting device fabricated using a lithography method, at least the light-emitting layer 113 (or the second light-emitting layer 113_2) and the layer(s) in the organic compound layer that is/are closer to the first electrode 101 than the light-emitting layer or the second light-emitting layer are formed by processing at the same time; consequently, their end portions are substantially aligned in the perpendicular direction.

The organic compound layer 103 may include another functional layer in addition to the light-emitting layer. FIG. 2A illustrates a structure where, in addition to the light-emitting layer 113, a hole-injection layer 111, a hole-transport layer 112, an electron-transport layer 114, and the electron-injection layer 115 are provided in the organic compound layer 103. Furthermore, the first light-emitting unit 501 and the second light-emitting unit 502 may each include another functional layer in addition to the light-emitting layer. FIG. 2B illustrates a structure where the hole-injection layer 111, a first hole-transport layer 112_1, and a first electron-transport layer 114_1, in addition to the first light-emitting layer 113_1, are provided in the first light-emitting unit 501 and a second hole-transport layer 1122, a second electron-transport layer 114_2, and the electron-injection layer 115, in addition to the second light-emitting layer 1132, are provided in the second light-emitting unit 502. The structure of the organic compound layer 103 in the present invention is not limited to these structures; any of the layers may be absent or another layer may be added. A carrier-blocking layer (a hole-blocking layer or an electron-blocking layer), an exciton-blocking layer, or the like may be typically added.

Then, components of the above light-emitting device 130, other than the intermediate layer 160, are described.

### <<Structure of First Electrode>>

The first electrode 101 includes an anode. The first electrode 101 may have a stacked-layer structure where the layer in contact with the organic compound layer 103 functions as the anode. The anode is preferably formed using any of metals, alloys, and conductive compounds with a high work function (specifically, higher than or equal to 4.0 eV), mixtures thereof, and the like. Specific examples include indium oxide-tin oxide (ITO: indium tin oxide), indium oxide-tin oxide containing silicon or silicon oxide, indium oxide-zinc oxide, and indium oxide containing tungsten oxide and zinc oxide (IWZO). Such conductive metal oxide films are usually formed by a sputtering method, but may be formed by application of a sol-gel method or the like. In an example of the formation method, a film of indium oxide-zinc oxide is formed by a sputtering method using a target obtained by adding 1 wt % to 20 wt % zinc oxide to indium oxide. Furthermore, a film of indium oxide containing tungsten oxide and zinc oxide (IWZO) can be formed by a sputtering method using a target in which 0.5 wt % to 5 wt % tungsten oxide and 0.1 wt % to 1 wt % zinc oxide are added to indium oxide. Alternatively, gold (Au), platinum (Pt), nickel (Ni), tungsten (W), chromium (Cr), molybdenum (Mo), iron (Fe), cobalt (Co), copper (Cu), palladium (Pd), a nitride of a metal material (e.g., titanium nitride), or the like can be used for the anode. Graphene can also be used for the anode. Note that an electrode material can be selected regardless of the work function when the second layer 162 in the above intermediate layer 160 is used for the layer (typically the hole-injection layer) in contact with the anode.

### <<Structure of Hole-Injection Layer>>

The hole-injection layer 111 is provided in contact with the anode and has a function of facilitating injection of holes into the organic compound layer 103 (the first light-emitting unit 501). The hole-injection layer 111 can be formed using phthalocyanine (abbreviation: H₂Pc), a phthalocyanine-based compound such as copper phthalocyanine (abbreviation: CuPc), an aromatic amine compound such as 4,4′-bis[N-(4-diphenylaminophenyl)-N-phenylamino]biphenyl (abbreviation: DPAB) or 4,4′-bis(N-{4-[N-(3-methylphenyl)-N′-phenylamino]phenyl}-N-phenylamino)biphenyl (abbreviation: DNTPD), or a high molecular compound such as poly(3,4-ethylenedioxythiophene)/poly(styrenesulfonic acid) (abbreviation: PEDOT/PSS).

The hole-injection layer 111 may be formed using a substance with an electron-accepting property. As the substance with an acceptor property, any of the substances described as the acceptor substance used for the second layer 162 of the above intermediate layer 160 can be used similarly.

The hole-injection layer 111 may be formed using the hole-transport material that is used for the second layer 162 of the above intermediate layer 160.

In the hole-injection layer 111, it is further preferable that the organic compound with a hole-transport property used in the composite material have a relatively deep HOMO level higher than or equal to −5.7 eV and lower than or equal to −5.4 eV. Using the organic compound with a hole-transport property which has a relatively deep HOMO level in the composite material makes it easy to inject holes into the hole-transport layer and to obtain a light-emitting device having a long lifetime. In addition, when the organic compound with a hole-transport property used in the composite material has a relatively deep HOMO level, induction of holes can be inhibited properly so that the light-emitting device can have a longer lifetime.

The formation of the hole-injection layer 111 can improve the hole-injection property, which allows the light-emitting device to be driven at a low voltage.

Among substances with an acceptor property, an organic compound with an acceptor property is easy to use because the organic compound is easily deposited by evaporation and its film is easily formed.

The second light-emitting unit 502 includes no hole-injection layer because the second layer 162 of the intermediate layer 160 functions as a hole-injection layer; however, the second light-emitting unit 502 may include a hole-injection layer.

The hole-transport layer (the first hole-transport layer 112_1 or the second hole-transport layer 112_2) includes an organic compound with a hole-transport property. The organic compound with a hole-transport property preferably has a hole mobility higher than or equal to I×10⁻⁶ cm²/Vs.

Examples of the aforementioned organic compound with a hole-transport property include the following compounds: compounds having an aromatic amine skeleton, such as 4,4′-bis[N-(1-naphthyl)-N-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-91-carbazole (abbreviation: BismBPCz), 9-(biphenyl-3-yl)-9′-(biphenyl-4-yl)-9H,9′H-3,3′-bicarbazole (abbreviation: mBPCCBP), 9-(2-naphthyl)-9′-phenyl-9H,9′H-3,3′-bicarbazole (abbreviation: βNCCP), 9-(3-biphenyl)-9′-(2-naphthyl)-3,3′-bi-9H-carbazole (abbreviation: βNCCmBP), 9-(4-biphenyl)-9′-(2-naphthyl)-3,3′-bi-9H-carbazole (abbreviation: βNCCBP), 9,9′-di-2-naphthyl-3,3′-9H,9′H-bicarbazole (abbreviation: BisβNCz), 9-(2-naphthyl)-9′-[1,1′:4′,1″-terphenyl]-3-yl-3,3′-9H,9′H-bicarbazole, 9-(2-naphthyl)-9′-[1,1′: 3′, 1″-terphenyl]-3-yl-3,3′-9H,9′H-bicarbazole, 9-(2-naphthyl)-9′-[1,1′: 3′, 1″-terphenyl]-5′-yl-3,3′-9H,9′H-bicarbazole, 9-(2-naphthyl)-9′-[1,1′: 4′, 1″-terphenyl]-4-yl-3,3′-9H,9′H-bicarbazole, 9-(2-naphthyl)-9′-[1,1:3′, 1″-terphenyl]-4-yl-3,3′-9H,9′H-bicarbazole, 9-(2-naphthyl)-9′-(triphenylen-2-yl)-3,3′-9H,9′H-bicarbazole, 9-phenyl-9′-(triphenylen-2-yl)-3,3′-9H,9′H-bicarbazole (abbreviation: PCCzTp), 9,9′-bis(triphenylen-2-yl)-3,3′-9H,9′H-bicarbazole, 9-(4-biphenyl)-9′-(triphenylen-2-yl)-3,3′-9H,9′H-bicarbazole, and 9-(triphenylen-2-yl)-9′-[1,1′: 3′, 1″-terphenyl]-4-yl-3,3′-9H,9′H-bicarbazole; compounds having a thiophene skeleton, such as 4,4′, 4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′, 4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in driving voltage. Note that any of the substances given as examples of the material with a hole-transport property that is used for the composite material in the hole-injection layer 111 can also be suitably used as the material included in the hole-transport layer.

### <<Structure of Light-Emitting Layer>>

The light-emitting layer (the light-emitting layer 113, the first light-emitting layer 113_1, or the second light-emitting layer 113_2) preferably includes a light-emitting substance and a host material. The light-emitting layer may additionally include other materials. Alternatively, the light-emitting layer may be a stack of two layers with different compositions.

As the light-emitting substance, fluorescent substances, phosphorescent substances, substances exhibiting thermally activated delayed fluorescence (TADF), or other light-emitting substances may be used.

Examples of the material that can be used as a fluorescent substance in the light-emitting layer are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-diphenyl-N,N′-bis[4-(9-phenyl-9H-fluoren-9-yl)phenyl]pyrene-1,6-diamine (abbreviation: 1,6FLPAPrn), N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis(N,N′,N″-triphenyl-1,4-phenylenediamine) (abbreviation: DPABPA), N,9-diphenyl-N-[4-(9,10-diphenyl-2-anthryl)phenyl]-9H-carbazol-3-armine (abbreviation: 2PCAPPA), N-[4-(9,10-diphenyl-2-anthryl)phenyl]-N,N′,N″-triphenyl-1,4-phenylenediamine (abbreviation: 2DPAPPA), N,N,N′,N′, N″,N″,N′″,N′″-octaphenyldibenzo[g,p]chrysene-2,7,10,15-tetraarine (abbreviation: DBC1), coumarin 30, N-(9,10-diphenyl-2-anthryl)-N,9-diphenyl-9H-carbazol-3-amine (abbreviation: 2PCAPA), N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), 9,10-bis(2-biphenyl)-2-(N,N′,N′-triphenyl-1,4-phenylenediamin-N-yl)anthracene (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), cournarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetranethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahvdro-1H,5H-benzo[ij]quinolizin-9-yl)ethenyl]-4H-pyran-4-ylidene}propanedinitrile (abbreviation: BisDCJTM), N,N′-diphenyl-N,N′-(1,6-pyrene-diyl)bis[(6-phenylbenzo[b]naphtho[1,2-d]furan)-8-amine](abbreviation: 1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Condensed aromatic diamine compounds typified by pyrenediamine compounds such as 1,6FLPAPrn, 1,6mMemFLPAPrn, and 1,6BnfAPrn-03 are particularly preferable because of their high hole-trapping properties and high emission efficiency or high reliability.

Examples of the material that can be used when a phosphorescent substance is used as the light-emitting substance in the light-emitting layer are as follows.

The examples include an organometallic iridium complex having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), and tris[4-(3-biphenyl)-5-isopropyl-3-phenyl-4H-1,2,4-triazolato]iridium(III) (abbreviation: [Ir(iPrptz-3b)₃]); an organometallic iridium complex having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Tr(Prptz1-Me)₃]); an organometallic iridium complex having an imidazole skeleton, such as fac-tris[1-(2,6-diisopropylphenyl)-2-phenyl-1H-imidazole]iridium(III) (abbreviation: [Ir(iPrpim)₃]) and tris[3-(2,6-dimethylphenyl)-7-methylimidazo[1,2-f]phenanthridinato]iridium(III) (abbreviation: [Ir(dmpimpt-Me)₃]); and an organometallic iridium complex in which a phenylpyridine derivative having an electron-withdrawing group is a ligand, such as bis[2-(4′, 6′-difluorophenyl)pyridinato-N,C^{2′}-]iridium(III) tetrakis(1-pyrazolyl)borate (abbreviation: FIr6), bis[2-(4′, 6′-difluorophenyl)pyridinato-N,C^{2′}]iridium(III) picolinate (abbreviation: Ffrpic), bis{2-[3′5′-bis(trifluoromethyl)phenyl]pyridinato-N,C^{2′}}iridium(III) picolinate (abbreviation: [Ir(CF₃ppy)₂(pic)]), and bis[2-(4′, 6′-difluorophenyl)pyridinato-N,C^{2′}]iridium(III) acetylacetonate (abbreviation: FIracac). These compounds emit blue phosphorescent light and have an emission peak in the wavelength range from 450 nm to 520 nm.

Other examples include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridiun(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)), {[2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: Ir(5mtpy-d₆)₂(mbfpypy-iPr-d₄)), [2-d₃-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d₃)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); and a rare earth metal complex such as tris(acetylacetonato)(monophenanthroline)terbium(III) (abbreviation: [Tb(acac)₃(Phen)]). These are mainly compounds that emit green phosphorescent light and have an emission peak in the wavelength range from 500 nm to 600 nm. Note that organometallic iridium complexes having a pyrimidine skeleton have distinctively high reliability or emission efficiency and thus are particularly preferable.

Other examples include organometallic iridium complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridiun(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpn)]); organometallic iridium complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes having a pyridine skeleton, such as tris(1-phenylisoquinolinato-N,C²)iridium(III) (abbreviation: [Ir(piq)₃]) and bis(I-phenylisoquinolinato-N,C^{2′})iridium(III) acetylacetonate (abbreviation: [Ir(piq)₂(acac)]); platinum complexes such as 2,3;7,8,12,13,17,18-octaethyl-21H,23H-porphyrinplatinum(II) (abbreviation: PtOEP); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds emit red phosphorescent light and have an emission peak in the wavelength range from 600 nm to 700 nm. Furthermore, the organometallic iridium complexes having a pyrazine skeleton can provide red light emission with favorable chromaticity.

Besides the above phosphorescent compounds, known phosphorescent compounds may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio 1)), and an octaethylporphyrin-platinum chloride complex (PtCl₂OEP), which are represented by the following structure formulae.

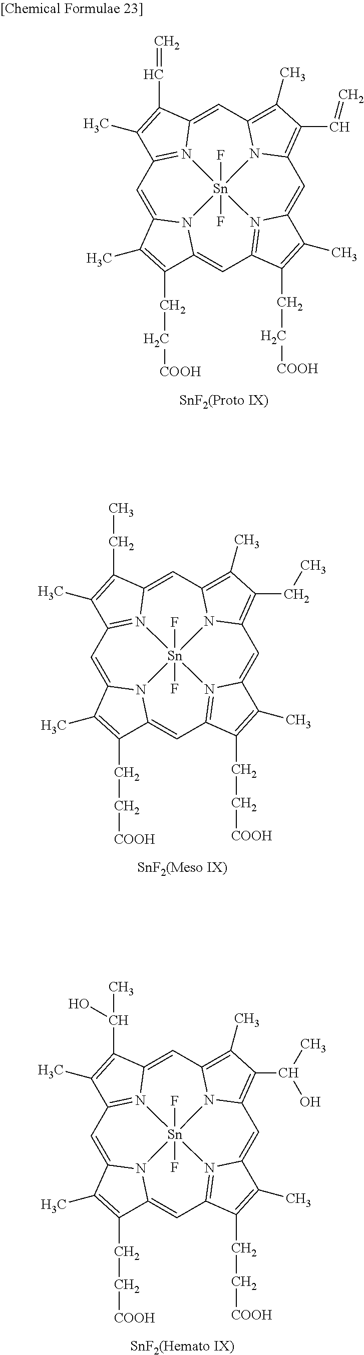

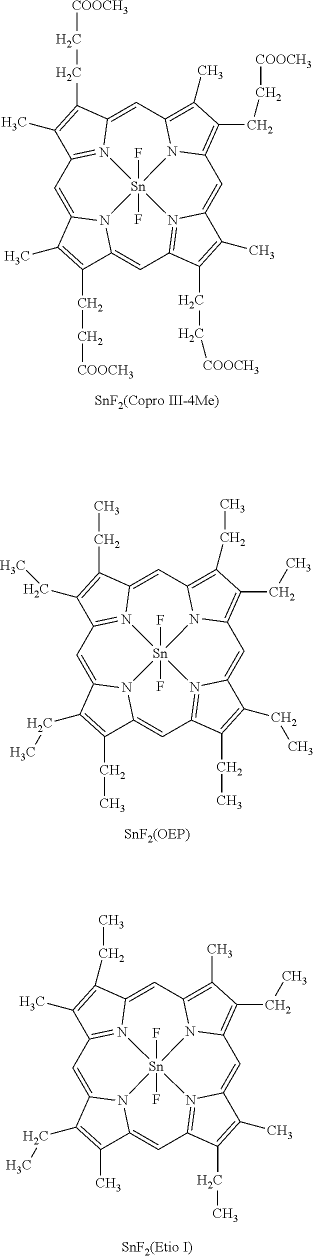

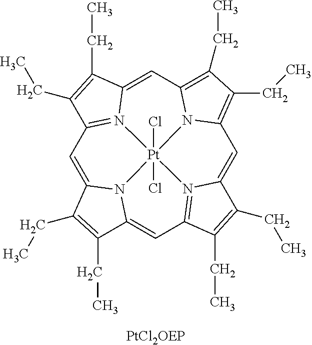

Alternatively, a heterocyclic compound having one or both of a π-electron rich heteroaromatic ring and a π-electron deficient heteroaromatic ring and represented by any of the following structure formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA) can be used. Such a heterocyclic compound is preferable because of having high electron-transport and hole-transport properties owing to a π-electron rich heteroaromatic ring and a π-electron deficient heteroaromatic ring. Among skeletons having a π-electron deficient heteroaromatic ring, a pyridine skeleton, a diazine skeleton (a pyrimidine skeleton, a pyrazine skeleton, and a pyridazine skeleton), and a triazine skeleton are preferable because of their high stability and reliability. In particular, a benzofuropyrirnidine skeleton, a benzothienopyrimidine skeleton, a benzofuropyrazine skeleton, and a benzothienopyrazine skeleton are preferable because of their high acceptor properties and high reliability. Among skeletons having a π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which a π-electron rich heteroaromatic ring is directly bonded to a π-electron deficient heteroaromatic ring is particularly preferable because the electron-donating property of the π-electron rich heteroaromatic ring and the electron-accepting property of the π-electron deficient heteroaromatic ring are both improved, the energy difference between the S₁ level and the T₁ level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a n-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a cyano group or a nitrile group such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

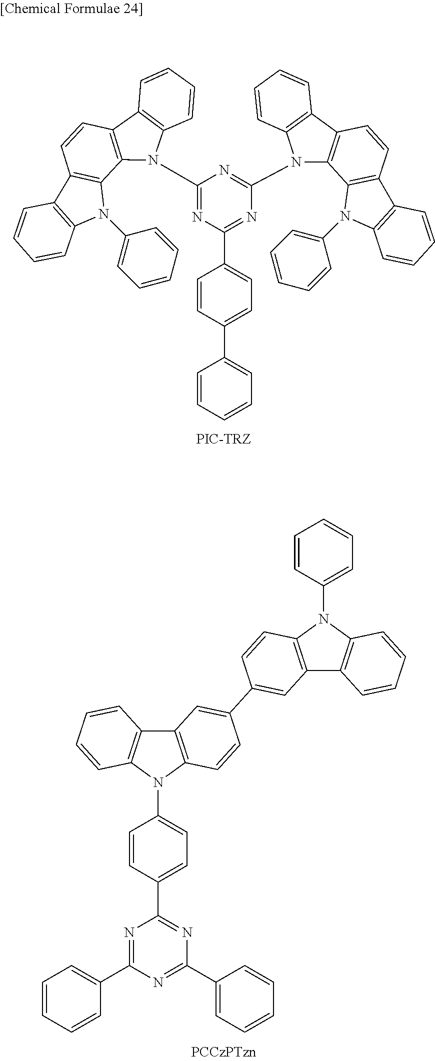 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

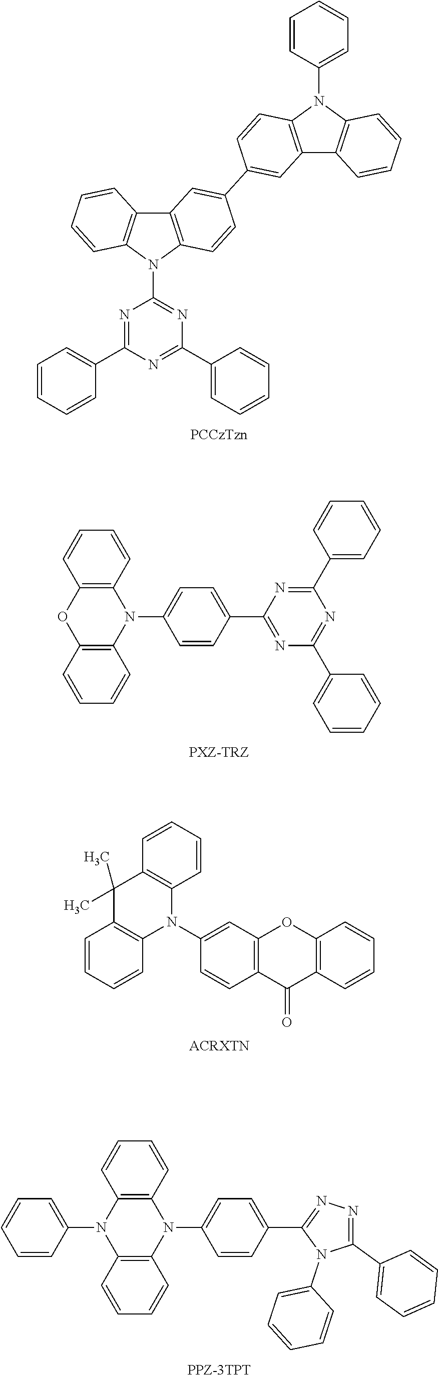 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3ccc(N4c5ccccc5Oc5ccccc54)cc3)n2)cc1 ; CC1(C)c2ccccc2N(c2ccc3c(=O)c4ccccc4oc3c2)c2ccccc21 ; c1ccc(-c2nnc(-c3ccc(N4c5ccccc5N(c5ccccc5)c5ccccc54)cc3)n2-c2ccccc2)cc1]

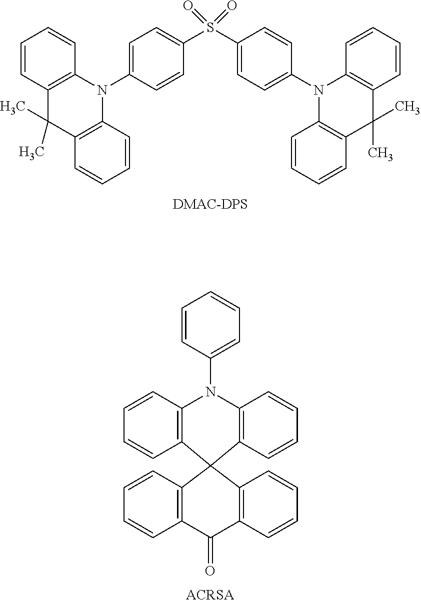 [SMILES: CC1(C)c2ccccc2N(c2ccc(S(=O)(=O)c3ccc(N4c5ccccc5C(C)(C)c5ccccc54)cc3)cc2)c2ccccc21 ; O=C1c2ccccc2C2(c3ccccc31)c1ccccc1N(c1ccccc1)c1ccccc12]

Alternatively, a TADF material whose singlet excited state and triplet excited state are in a thermal equilibrium state may be used. Such a TADF material has a short emission lifetime (excitation lifetime), inhibiting a decrease in the efficiency of a light-emitting device in a high-luminance region. Specifically, a material having the following molecular structure can be used.

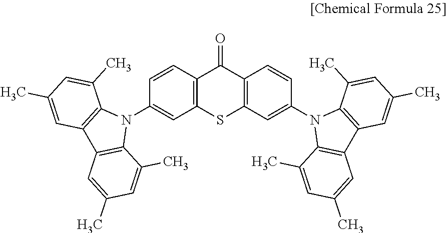

Note that a TADF material is a material having a small difference between the S₁ level and the T₁ level and a function of converting triplet excitation energy into singlet excitation energy by reverse intersystem crossing. Thus, a TADF material enables upconversion of triplet excitation energy into singlet excitation energy (i.e., reverse intersystem crossing) using a small amount of thermal energy and can efficiently generate a singlet excited state. In addition, the triplet excitation energy can be converted into light emission.

An exciplex whose excited state is formed by two kinds of substances has an extremely small difference between the S₁ level and the T₁ level and functions as a TADF material capable of converting triplet excitation energy into singlet excitation energy.

A phosphorescent spectrum observed at a low temperature (e.g., 77 K to 10 K) is used for an index of the T₁ level. When the level of energy with a wavelength of the line obtained by extrapolating a tangent to the fluorescent spectrum at a tail on the short wavelength side is the Si level and the level of energy with a wavelength of the line obtained by extrapolating a tangent to the phosphorescent spectrum at a tail on the short wavelength side is the T₁ level, the difference between the S₁ level and the T₁ level of the TADF material is preferably smaller than or equal to 0.3 eV, further preferably smaller than or equal to 0.2 eV.

When a TADF material is used as the light-emitting substance, the S₁ level of the host material is preferably higher than that of the TADF material. In addition, the T₁ level of the host material is preferably higher than that of the TADF material.

As the host material in the light-emitting layer, any of various carrier-transport materials such as materials with an electron-transport property and/or materials with a hole-transport property, and the TADF materials can be used.

As the material with a hole-transport property, the aforementioned material given as the material with a hole-transport property can be used similarly.

As the material with an electron-transport property, the aforementioned material given as the material with an electron-transport property can be used similarly.

As the TADF material that can be used as the host material, the aforementioned materials given as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S₁ level of the TADF material is preferably higher than that of the fluorescent substance in order that high emission efficiency can be achieved. Furthermore, the T₁ level of the TADF material is preferably higher than the Si level of the fluorescent substance. Therefore, the T1 level of the TADF material is preferably higher than that of the fluorescent substance.

It is also preferable to use a TADF material emitting light whose wavelength overlaps with the wavelength on the lowest-energy-side absorption band of the fluorescent substance, in which case excitation energy is transferred smoothly from the TADF material to the fluorescent substance and light emission can be obtained efficiently.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton that causes light emission) of the fluorescent substance. As the protective group, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be made away from each other with little influence on carrier transport or carrier recombination. Here, the luminophore refers to an atomic group (skeleton) that causes light emission in a fluorescent substance. The luminophore is preferably a skeleton having a π bond, further preferably includes an aromatic ring, and still further preferably includes a condensed aromatic ring or a condensed heteroarornatic ring. Examples of such a luminophore include a phenanthrene skeleton, a stilbene skeleton, an acridone skeleton, a phenoxazine skeleton, a phenothiazine skeleton, a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

In the case where a fluorescent substance is used as the light-emitting substance, a material having an anthracene skeleton is suitably used as the host material. The use of a substance having an anthracene skeleton as the host material for the fluorescent substance makes it possible to obtain a light-emitting layer with high emission efficiency and high durability. Among the substances having an anthracene skeleton, a substance having a diphenylanthracene skeleton, in particular, a substance having a 9,10-diphenylanthracene skeleton, is chemically stable and thus is preferably used as the host material. The host material preferably has a carbazole skeleton because the hole-injection and hole-transport properties are improved; further preferably, the host material has a benzocarbazole skeleton in which a benzene ring is further condensed to a carbazole skeleton because the HOMO level thereof is shallower than that of the host material having a carbazole skeleton by approximately 0.1 eV and thus holes enter the host material easily. In particular, the host material preferably has a dibenzocarbazole skeleton, because the HOMO level of the host material having a dibenzocarbazole skeleton is shallower than that of the host material having a carbazole skeleton by approximately 0.1 eV, the host material having a dibenzocarbazole skeleton is thus easier for holes to enter than the host material having a carbazole skeleton, and the host material having a dibenzocarbazole skeleton has a higher hole-transport property and higher heat resistance than the host material having a carbazole skeleton. Accordingly, a substance having both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Examples of such a substance include 9-phenyl-3-[4-(10-phenyl-9-anthryl)phenyl]-9H-carbazole (abbreviation: PCzPA), 3-[4-(1-naphthyl)phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7I-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4′-(9-phenyl-9H-fluoren-9-yl)biphenyl-4-yl]anthracene (abbreviation: FLPPA), 9-(1-naphthyl)-10-[4-(2-naphthyl)phenyl]anthracene (abbreviation: αN-βNPAnth), 9-(I-naphthyl)-10-(2-naphthyl)anthracene (abbreviation: α,β-ADN), 2-(10-phenylanthracen-9-yl)dibenzofuran, 2-(10-phenyl-9-anthracenyl)benzo[b]naphtho[2,3-d]furan (abbreviation: Bnf(II)PhA), 9-(2-naphthyl)-10-[3-(2-naphthyl)phenyl]anthracene (abbreviation: βN-mβNPAnth), and 1-{4-[10-(biphenyl-4-yl)-9-anthracenyl]phenyl}-2-ethyl-1H-benzimidazole (abbreviation: EtBImPBPhA). In particular, CzPA, cgDBCzPA, 2mBnfPPA, and PCzPA have excellent characteristics and thus are preferably selected.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material having a hole-transport property to the content of the material having an electron-transport property may be 1:19 to 19:1.

Note that a phosphorescent substance can be used as part of the mixed material. When a fluorescent substance is used as the light-emitting substance, a phosphorescent substance can be used as an energy donor supplying excitation energy to the fluorescent substance.

An exciplex may be formed by these mixed materials. These mixed materials are preferably selected so as to form an exciplex that exhibits light emission whose wavelength overlaps with the wavelength on the lowest-energy-side absorption band of the light-emitting substance, in which case energy can be transferred smoothly and light emission can be obtained efficiently. Such a structure is preferably used to reduce the driving voltage.

Note that at least one of the materials forming an exciplex may be a phosphorescent substance. In this case, triplet excitation energy can be efficiently converted into singlet excitation energy by reverse intersystem crossing.

Combination of a material having an electron-transport property and a material having a hole-transport property whose HOMO level is higher than or equal to that of the material having an electron-transport property is preferable for forming an exciplex efficiently. In addition, the LUMO level of the material having a hole-transport property is preferably higher than or equal to that of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed by a phenomenon in which the emission spectrum of the mixed film in which the material having a hole-transport property and the material having an electron-transport property are mixed is shifted to the longer wavelength than the emission spectrum of each of the materials (or has another peak on the longer wavelength side) observed by comparison of the emission spectra of the material having a hole-transport property, the material having an electron-transport property, and the mixed film of these materials, for example. Alternatively, the formation of an exciplex can be confirmed by a difference in transient response, such as a phenomenon in which the transient photoluminescence (PL) lifetime of the mixed film has longer lifetime components or has a larger proportion of delayed components than that of each of the materials, observed by comparison of transient PL of the material having a hole-transport property, the material having an electron-transport property, and the mixed film of these materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of these materials.

### <<Structure of Electron-Transport Layer>

The electron-transport layer (the electron-transport layer 114, the first electron-transport layer 114_1, or the second electron-transport layer 1142) includes a substance with an electron-transport property. The substance with an electron-transport property is preferably a substance having an electron mobility higher than or equal to 1×10⁻⁷ cm²/Vs, further preferably higher than or equal to 1×10⁻⁶ cm²/Vs, when the square root of electric field strength [V/cm] is 600. Note that any other substance can be used as long as the substance has an electron-transport property higher than a hole-transport property. An organic compound having a π-electron deficient heteroaromatic ring is preferable as the above organic compound. The organic compound having a π-electron deficient heteroaromatic ring is preferably one or more of an organic compound having a heteroaromatic ring with an azole skeleton, an organic compound having a heteroaromatic ring with a pyridine skeleton, an organic compound having a heteroaromatic ring with a diazine skeleton, and an organic compound having a heteroaromatic ring with a triazine skeleton.

The organic compound of one embodiment of the present invention described in Embodiment 1 can be used for the electron-transport layer. The organic compound with an electron-transport property used in the first layer of the above intermediate layer 160 can also be used for the electron-transport layer. Among the above-described materials, the organic compound that has a heteroaromatic ring having a diazine skeleton, the organic compound that has a heteroaromatic ring having a pyridine skeleton, and the organic compound that has a heteroaromatic ring having a triazine skeleton are especially preferable because of having high reliability. In particular, the organic compound that has a heteroaromatic ring having a diazine (pyrimidine or pyrazine) skeleton and the organic compound that has a heteroaromatic ring having a triazine skeleton have a high electron-transport property to contribute to a reduction in driving voltage.

The electron mobility of the electron-transport layer is preferably higher than or equal to 1×10⁻⁷ cm²/Vs and lower than or equal to 5×10⁻⁵ cm²/Vs, when the square root of the electric field strength [V/cm] is 600. The amount of electrons injected into the light-emitting layer can be controlled by the reduction in the electron-transport property of the electron-transport layer, whereby the light-emitting layer can be prevented from having excess electrons. It is particularly preferable to employ this structure when the hole-injection layer is formed using a composite material that includes a material having a hole-transport property with a relatively deep HOMO level higher than or equal to −5.7 eV and lower than or equal to −5.4 eV, in which case a long lifetime can be achieved. In this case, the material with an electron-transport property preferably has a HOMO level higher than or equal to −6.0 eV.

### <<Structure of Electron-Injection Layer>>

As the electron-injection layer 115, a layer that includes an alkali metal, an alkaline earth metal, a rare earth metal, a compound of an alkali metal, an alkaline earth metal, or a rare earth metal, or a complex of an alkali metal, an alkaline earth metal, or a rare earth metal, such as lithium fluoride (LiF), cesium fluoride (CsF), calcium fluoride (CaF₂), 8-quinolinolato-lithium (abbreviation: Liq), or ytterbium (Yb) may be provided. An electride or a layer that is formed using a substance with an electron-transport property and that includes an alkali metal, an alkaline earth metal, or a compound of an alkali metal or an alkaline earth metal may be used as the electron-injection layer 115. Examples of the electride include a substance in which electrons are added at a high concentration to calcium oxide-aluminum oxide.

Note that as the electron-injection layer 115, it is possible to use a layer including a substance that has an electron-transport property (preferably an organic compound having a bipyridine skeleton) and includes a fluoride of the alkali metal or the alkaline earth metal at a concentration higher than or equal to that at which the electron-injection layer 115 becomes in a microcrystalline state (50 wt % or higher). Since the layer has a low refractive index, a light-emitting device including the layer can have high external quantum efficiency.

The organic compound of one embodiment of the present invention described in Embodiment 1 can be used for the electron-injection layer 115. The first layer including the organic compound of one embodiment of the present invention described in Embodiment 2 can be used for the electron-injection layer 115.

### <<Structure of Second Electrode>

The second electrode 102 includes a cathode. The second electrode 102 may have a stacked-layer structure where the layer in contact with the organic compound layer 103 functions as the cathode. As a substance of the cathode, any of metals, alloys, and electrically conductive compounds with a low work function (specifically, lower than or equal to 3.8 eV), mixtures thereof, and the like can be used. Specific examples of such a cathode material include elements belonging to Group 1 and Group 2 of the periodic table, such as alkali metals (e.g., lithium (Li) or cesium (Cs)), magnesium (Mg), calcium (Ca), and strontium (Sr), alloys containing any of these elements (e.g., MgAg and AlLi), rare earth metals such as europium (Eu) and ytterbium (Yb), and alloys containing any of these rare earth metals. However, when the electron-injection layer is provided between the second electrode 102 and the electron-transport layer, any of a variety of conductive materials such as Al, Ag, ITO, or indium oxide-tin oxide containing silicon or silicon oxide can be used for the cathode regardless of the work function.

When the second electrode 102 is formed using a material transmitting visible light, the light-emitting device can emit light from the second electrode 102 side.

Films of these conductive materials can be formed by a dry process such as a vacuum evaporation method or a sputtering method, an ink-jet method, a spin coating method, or the like. Alternatively, a wet process using a sol-gel method or a wet process using a paste of a metal material may be employed.

The organic compound layer 103 can be formed by any of a variety of methods, including a dry process and a wet process. For example, a vacuum evaporation method, a gravure printing method, an offset printing method, a screen printing method, an ink-jet method, a spin coating method, or the like may be used.

Different methods may be used to form the electrodes or the layers described above.

This embodiment can be combined as appropriate with any of the other embodiments and examples. In this specification, in the case where a plurality of structure examples are shown in one embodiment, the structure examples can be combined as appropriate.

### Embodiment 4

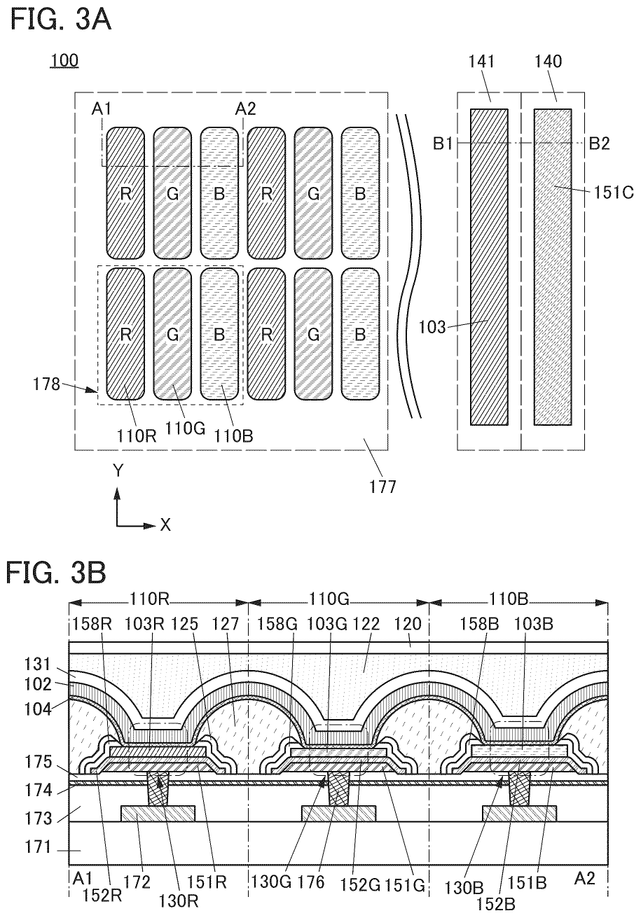

As illustrated in FIGS. 3A and 3B, the light-emitting devices 130 constitute a display apparatus by multiple providing on the insulating layer 175. In this embodiment, the display apparatus of one embodiment of the present invention will be described in detail.

A display apparatus 100 includes a pixel portion 177 in which a plurality of pixels 178 are arranged in matrix. The pixel 178 includes a subpixel 110R, a subpixel 110G, and a subpixel 110B.

In this specification and the like, for example, description common to the subpixels 110R, 110G, and 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and yellow (Y), and four subpixels emitting light of R, G, and B and infrared (IR) light.

In this specification and the like, the row direction and the column direction are sometimes referred to as the X direction and the Y direction, respectively. The X direction and the Y direction intersect with each other and are perpendicular to each other, for example.

FIG. 3A illustrates an example where subpixels of different colors are arranged in the X direction and subpixels of the same color are arranged in the Y direction. Note that subpixels of different colors may be arranged in the Y direction, and subpixels of the same color may be arranged in the X direction.

A connection portion 140 and a region 141 may be provided outside the pixel portion 177, and the region 141 is provided between the pixel portion 177 and the connection portion 140. The organic compound layer 103 is provided in the region 141. A conductive layer 151C is provided in the connection portion 140.

Although FIG. 3A illustrates an example where the region 141 and the connection portion 140 are located on the right side of the pixel portion 177, there is no particular limitation on the positions of the region 141 and the connection portion 140. The number of regions 141 and the number of connection portions 140 can each be one or more.

FIG. 3B is a cross-sectional view along the dashed-dotted line A1-A2 in FIG. 3A. As illustrated in FIG. 3B, the display apparatus 100 includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is provided over a substrate (not illustrated). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A protective layer 131 is provided to cover the light-emitting device 130. A substrate 120 is attached to the protective layer 131 with a resin layer 122. An inorganic insulating layer 125 and an insulating layer 127 over the inorganic insulating layer 125 are preferably provided between the adjacent light-emitting devices 130.

Although each of the inorganic insulating layer 125 and the insulating layer 127 looks like a plurality of layers in the cross-sectional view in FIG. 3B, each of the inorganic insulating layer 125 and the insulating layer 127 is preferably one continuous layer when the display apparatus 100 is seen from above. In other words, the inorganic insulating layer 125 and the inorganic insulating layer 127 preferably include opening portions over first electrodes.

In FIG. 3B, a light-emitting device 130R, a light-emitting device 130G, and a light-emitting device 130B are each illustrated as the light-emitting device 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, 130G, or 130B may emit visible light of another color or infrared light.

The display apparatus of one embodiment of the present invention can be, for example, a top-emission display apparatus where light is emitted in the direction opposite to a substrate over which light-emitting devices are formed. Note that the display apparatus of one embodiment of the present invention may be of a bottom-emission type.

Examples of a light-emitting substance included in the light-emitting device 130 include organic compounds or organometallic complexes such as a substance emitting fluorescent light (a fluorescent material), a substance emitting phosphorescent light (a phosphorescent material), and a substance exhibiting thermally activated delayed fluorescence (a thermally activated delayed fluorescent (TADF) material). Other examples include inorganic compounds (e.g., a quantum dot material).

The light-emitting device 130R has a structure as described in Embodiments 2 and 3. The light-emitting device 130R includes the first electrode (pixel electrode) including a conductive layer 151R and a conductive layer 152R, an organic compound layer 103R over the first electrode, a common layer 104 over the organic compound layer 103R, and a second electrode (common electrode) 155 over the common layer 104. Although the common layer 104 is not necessarily provided, it is preferable to provide the common layer 104 to reduce damage to the organic compound layer 103R during processing. In the case where the common layer 104 is provided, the common layer 104 is preferably an electron-injection layer. Furthermore, in the case where the common layer 104 is provided, a stack of the organic compound layer 103R and the common layer 104 corresponds to the organic compound layer 103 described in Embodiments 2 and 3.

The light-emitting device 130G has a structure as described in Embodiments 2 and 3. The light-emitting device 130G includes the first electrode (pixel electrode) including a conductive layer 151G and a conductive layer 152G, an organic compound layer 103G over the first electrode, the common layer 104 over the organic compound layer 103G, and the second electrode (common electrode) 155 over the common layer 104. Although the common layer 104 is not necessarily provided, it is preferable to provide the common layer 104 to reduce damage to the organic compound layer 103G during processing. In the case where the common layer 104 is provided, the common layer 104 is preferably an electron-injection layer. Furthermore, in the case where the common layer 104 is provided, a stack of the organic compound layer 103G and the common layer 104 corresponds to the organic compound layer 103 described in Embodiments 2 and 3.

The light-emitting device 130B has a structure as described in Embodiments 2 and 3. The light-emitting device 130B includes the first electrode (pixel electrode) including a conductive layer 151B and a conductive layer 152B, an organic compound layer 103B over the first electrode, the common layer 104 over the organic compound layer 103B, and the second electrode (common electrode) 155 over the common layer 104. Although the common layer 104 is not necessarily provided, it is preferable to provide the common layer 104 to reduce damage to the organic compound layer 103B during processing. In the case where the common layer 104 is provided, the common layer 104 is preferably an electron-injection layer. Furthermore, in the case where the common layer 104 is provided, a stack of the organic compound layer 103B and the common layer 104 corresponds to the organic compound layer 103 described in Embodiments 2 and 3.

In the light-emitting device, one of the pixel electrode and the common electrode functions as an anode and the other functions as a cathode. Hereinafter, description is made on the assumption that the pixel electrode functions as the anode and the common electrode functions as the cathode unless otherwise specified.

The organic compound layers 103R, the organic compound layers 103G, and the organic compound layers 103B are island-shaped layers that are separate from each other; alternatively, an organic compound layer of the light-emitting devices of one emission color may be separate from an organic compound layer of the light-emitting devices of another emission color. Providing the island-shaped organic compound layer 103 in each of the light-emitting devices 130 can inhibit leakage current between the adjacent light-emitting devices 130 even in a high-resolution display apparatus. This can prevent crosstalk, so that a display apparatus with extremely high contrast can be obtained. Specifically, a display apparatus having high current efficiency at low luminance can be obtained.

The island-shaped organic compound layer 103 is formed by forming an EL film and processing the EL film by a lithography method.

In the display apparatus of one embodiment of the present invention, the first electrode (pixel electrode) of the light-emitting device preferably has a stacked-layer structure. For example, in the example illustrated in FIG. 3B, the first electrode of the light-emitting device 130 is a stack of the conductive layer 151 and the conductive layer 152. In the case where the display apparatus 100 is of a top-emission type and the pixel electrode of the light-emitting device 130 functions as the anode, for example, the conductive layer 151 preferably has high visible light reflectance, and the conductive layer 152 preferably has a visible-light-transmitting property and a high work function. In the case where the display apparatus 100 is of a top-emission type, the higher the visible light reflectance of the pixel electrode is, the higher the efficiency of extraction of the light emitted by the organic compound layer 103 is. In the case where the pixel electrode functions as the anode, the higher the work function of the pixel electrode is, the easier hole injection into the organic compound layer 103 is. Accordingly, when the pixel electrode of the light-emitting device 130 is a stack of the conductive layer 151 with high visible light reflectance and the conductive layer 152 with a high work function, the light-emitting device 130 can have high light extraction efficiency and a low driving voltage.

In the case where the conductive layer 151 has high visible light reflectance, the visible light reflectance of the conductive layer 151 is preferably higher than or equal to 40% and lower than or equal to 100%, further preferably higher than or equal to 70% and lower than or equal to 100%, for example. When used as an electrode having a visible-light-transmitting property, the conductive layer 152 preferably has a visible light transmittance higher than or equal to 40%, for example.

Here, such a pixel electrode being a stack composed of a plurality of layers might change in quality as a result of, for example, a reaction between the plurality of layers. For example, when a film formed after the formation of the pixel electrode is removed by a wet etching method, contact of a chemical solution with the pixel electrode might cause galvanic corrosion.

In view of the above, the conductive layer 152 is formed to cover the top surface and the side surface of the conductive layer 151 in the display apparatus 100 of this embodiment. This can inhibit a chemical solution from coming into contact with the conductive layer 151 when a film that is formed after formation of the pixel electrode including the conductive layer 151 and the conductive layer 152 is removed by a wet etching method, for example. Accordingly, occurrence of galvanic corrosion in the pixel electrode can be inhibited, for example. This allows the display apparatus 100 to be manufactured by a high-yield method and to be accordingly inexpensive. In addition, generation of a defect in the display apparatus 100 can be inhibited, which makes the display apparatus 100 highly reliable.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, indium tin oxide, indium zinc oxide, zinc oxide, zinc oxide containing gallium, titanium oxide, indium zinc oxide containing gallium, indium zinc oxide containing aluminum, indium tin oxide containing silicon, indium zinc oxide containing silicon, and the like. In particular, indium tin oxide containing silicon can be suitably used for the conductive layer 152 because of having a high work function, for example, a work function higher than or equal to 4.0 eV.

The conductive layer 151 and the conductive layer 152 may each be a stack of a plurality of layers that include different materials. In this case, the conductive layer 151 may include a layer formed using a material that can be used for the conductive layer 152, such as a conductive oxide. Furthermore, the conductive layer 152 may include a layer formed using a material that can be used for the conductive layer 151, such as a metal material. In the case where the conductive layer 151 is a stack of two or more layers, for example, a layer in contact with the conductive layer 152 can be formed using a material that can be used for the conductive layer 152.

The conductive layer 151 preferably has an end portion with a tapered shape. Specifically, the end portion of the conductive layer 151 preferably has a tapered shape with a taper angle less than 90°. In that case, the conductive layer 152 provided along the side surface of the conductive layer 151 also has a tapered shape. When the side surface of the conductive layer 152 has a tapered shape, coverage with the organic compound layer 103 provided along the side surface of the conductive layer 152 can be improved.

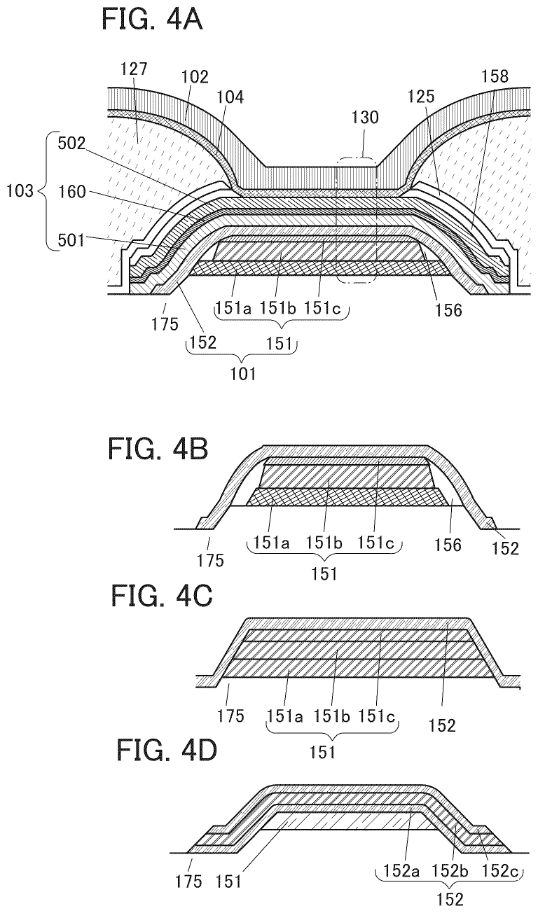

FIG. 4A illustrates the cases where the conductive layer 151 has a stacked-layer structure of a plurality of layers that include different materials. As illustrated in FIG. 4A, the conductive layer 151 includes a conductive layer 151a, a conductive layer 151b over the conductive layer 151a, and a conductive layer 151c over the conductive layer 151b. In other words, the conductive layer 151 illustrated in FIG. 4A has a three-layer structure. In the case where the conductive layer 151 has a stacked-layer structure of a plurality of layers as described above, the visible light reflectance of at least one of the layers included in the conductive layer 151 is made higher than that of the conductive layer 152.

In the example illustrated in FIG. 4A, the conductive layer 151b is sandwiched between the conductive layers 151a and 151c. A material that is less likely to change in quality than the material for the conductive layer 151b is preferably used for the conductive layers 151a and 151c. The conductive layer 151a can be formed using, for example, a material that is less likely to migrate owing to contact with the insulating layer 175 than the material for the conductive layer 151b. The conductive layer 151c can be formed using a material an oxide of which has lower electrical resistivity than an oxide of the material for the conductive layer 151b and which is less likely to be oxidized than the material for the conductive layer 151b.

In this manner, the structure where the conductive layer 151b is sandwiched between the conductive layers 151a and 151c can expand the range of choices for the material for the conductive layer 151b. The conductive layer 151b, for example, can thus have higher visible light reflectance than at least one of the conductive layers 151a and 151c. For example, aluminum can be used for the conductive layer 151b. The conductive layer 151b may be formed using an alloy containing aluminum. The conductive layer 151a can be formed using titanium; titanium has lower visible light reflectance than aluminum but is less likely to migrate owing to contact with the insulating layer 175 than aluminum. Furthermore, the conductive layer 151c can be formed using titanium; titanium is less likely to be oxidized than aluminum and an oxide of titanium has lower electrical resistivity than aluminum oxide, although titanium has lower visible light reflectance than aluminum.

The conductive layer 151c may be formed using silver or an alloy containing silver, Silver is characterized by its visible light reflectance higher than that of titanium. In addition, silver is characterized by being less likely to be oxidized than aluminum, and silver oxide is characterized by having electrical resistivity lower than that of aluminum oxide, Thus, the conductive layer 151c formed using silver or an alloy containing silver can favorably increase the visible light reflectance of the conductive layer 151 and inhibit an increase in the electrical resistance of the pixel electrode due to oxidation of the conductive layer 151b. Here, as the alloy containing silver, an alloy of silver, palladium, and copper (also referred to as Ag—Pd—Cu or APC) can be used, for example. When the conductive layer 151c is formed using silver or an alloy containing silver and the conductive layer 151b is formed using aluminum, the visible light reflectance of the conductive layer 151c can be higher than that of the conductive layer 151b. Here, the conductive layer 151b may be formed using silver or an alloy containing silver. The conductive layer 151a may be formed using silver or an alloy containing silver.

Meanwhile, a film formed using titanium has better processability in etching than a film formed using silver. Thus, use of titanium for the conductive layer 151c can facilitate formation of the conductive layer 151c. Note that a film formed using aluminum also has better processability in etching than a film formed using silver.

The conductive layer 151 having a stacked-layer structure of a plurality of layers as described above can improve the characteristics of the display apparatus. For example, the display apparatus 100 can have high light extraction efficiency and high reliability.

Here, in the case where the light-emitting device 130 has a microcavity structure, use of silver or an alloy containing silver, i.e., a material with high visible light reflectance, for the conductive layer 151c can favorably increase the light extraction efficiency of the display apparatus 100.

As already described above, the conductive layer 151 preferably has a side surface with a tapered shape. Specifically, the side surface of the conductive layer 151 preferably has a tapered shape with a taper angle less than 90°. For example, in the conductive layer 151 illustrated in FIG. 4A, the side surface of at least one of the conductive layer 151a, the conductive layer 151b, and the conductive layer 151c preferably has a tapered shape.

The conductive layer 151 illustrated in FIG. 4A can be formed by a lithography method. Specifically, first, a conductive film to be the conductive layer 151a, a conductive film to be the conductive layer 151b, and a conductive film to be the conductive layer 151c are sequentially formed. Next, a resist mask is formed over the conductive film to be the conductive layer 151c. Then, the conductive film in the region not overlapping with the resist mask is removed by etching. Here, when the conductive film is processed under conditions where the resist mask is easily recessed (reduced in size) as compared to the case where the conductive layer 151 is formed such that the side surface does not have a tapered shape (i.e., the conductive layer 151 is formed to have a perpendicular side surface), the side surface of the conductive layer 151 can have a tapered shape.

Here, when the conductive film is processed under conditions where the resist mask is easily recessed (reduced in size), the conductive film might be easily processed in the horizontal direction. That is, the etching sometimes might become isotropic as compared to the case where the conductive layer 151 is formed to have a perpendicular side surface.

In the case where the conductive layer 151 is a stack of a plurality of layers formed of different materials, the plurality of layers sometimes differ in processability in the horizontal direction. For example, the conductive layer 151a, the conductive layer 151b, and the conductive layer 151c sometimes differ in processability in the horizontal direction.

In that case, after the processing of the conductive film, as illustrated in FIG. 4A, the side surface of the conductive layer 151b may be positioned inward from the side surfaces of the conductive layers 151a and 151c and a protruding portion may be formed. This might impair coverage of the conductive layer 151 with the conductive layer 152 and cause step disconnection of the conductive layer 152.

In view of this, an insulating layer 156 is preferably provided as illustrated in FIG. 4A. FIG. 4A illustrates an example where the insulating layer 156 is provided over the conductive layer 151a to include a region overlapping with the side surface of the conductive layer 151b. In this structure, occurrence of step disconnection or thinning of the conductive layer 152 due to the protruding portion can be inhibited, so that poor connection or an increase in driving voltage can be inhibited.

Although FIG. 4A illustrates the structure where the side surface of the conductive layer 151b is entirely covered with the insulating layer 156, part of the side surface of the conductive layer 151b is not necessarily covered with the insulating layer 156. Also in a pixel electrode with a later-described structure, part of the side surface of the conductive layer 151b is not necessarily covered with the insulating layer 156.

In the case where the conductive layer 151 has the structure illustrated in FIG. 4A, the conductive layer 152 is provided to cover the conductive layers 151a, 151b, and 151c and the insulating layer 156 and to be electrically connected to the conductive layers 151a, 151b, and 151c. This can prevent a chemical solution from coming into contact with the conductive layers 151a, 151b, and 151c when a film formed after formation of the conductive layer 152 is removed by a wet etching method, for example. It is thus possible to inhibit occurrence of corrosion in the conductive layers 151a, 151b, and 151c. Hence, the display apparatus 100 can be fabricated by a high-yield method. Moreover, the display apparatus 100 can have high reliability since generation of defects is inhibited therein.

Here, the insulating layer 156 preferably has a curved surface as illustrated in FIG. 4A. In that case, step disconnection in the conductive layer 152 covering the insulating layer 156 is less likely to occur than in the case where the insulating layer 156 has a perpendicular side surface (a side surface parallel to the Z direction), for example. In addition, step disconnection in the conductive layer 152 covering the insulating layer 156 is less likely to occur also in the case where the side surface of the insulating layer 156 has a tapered shape, or specifically, a tapered shape with a taper angle less than 90°, than in the case where the insulating layer 156 has a perpendicular side surface, for example. As described above, the display apparatus 100 can be fabricated by a high-yield method. Moreover, the display apparatus 100 can have high reliability since generation of defects is inhibited therein.

FIG. 4A illustrates the structure where the side surface of the conductive layer 151b is located inward from that of the conductive layer 151a and that of the conductive layer 151c; however, one embodiment of the present invention is not limited to this structure. For example, the side surface of the conductive layer 151b may be located outward from that of the conductive layer 151a. The side surface of the conductive layer 151b may be located outward from that of the conductive layer 151c.

FIGS. 4B to 4D illustrate other structures of the first electrode 101. FIG. 4B illustrates a variation structure of the first electrode 101 in FIG. 4A, in which the insulating layer 156 covers the side surfaces of the conductive layers 151a, 151b, and 151c instead of covering only the side surface of the conductive layer 151b.

FIG. 4C illustrates a variation structure of the first electrode 101 in FIG. 4A, in which the insulating layer 156 is not provided.

FIG. 4D illustrates a variation structure of the first electrode 101 in FIG. 4A, in which the conductive layer 151 does not have a stacked-layer structure but the conductive layer 152 has a stacked-layer structure.

A conductive layer 152a has higher adhesion to a conductive layer 152b than the insulating layer 175 does, for example. For the conductive layer 152a, an oxide containing one or more selected from indium, tin, zinc, gallium, titanium, aluminum, and silicon, for example, can be used. For example, it is preferable to use a conductive oxide containing one or more of indium oxide, indium tin oxide, indium zinc oxide, zinc oxide, zinc oxide containing gallium, titanium oxide, indium titanium oxide, zinc titanate, aluminum zinc oxide, indium zinc oxide containing gallium, indium zinc oxide containing aluminum, indium tin oxide containing silicon, indium zinc oxide containing silicon, and the like. Accordingly, peeling of the conductive layer 152b can be inhibited. The conductive layer 152b is not in contact with the insulating layer 175.

The conductive layer 152b is a layer whose visible light reflectance (e.g., reflectance with respect to light with a predetermined wavelength longer than or equal to 400 nm and shorter than 750 nm) is higher than that of the conductive layers 151, 152a, and 152c. The visible light reflectance of the conductive layer 152b can be, for example, higher than or equal to 70% and lower than or equal to 100%, and is preferably higher than or equal to 80% and lower than or equal to 100%, further preferably higher than or equal to 90% and lower than or equal to 100%. For the conductive layer 152b, a material having higher visible light reflectance than aluminum can be used, for example. Specifically, for the conductive layer 152b, silver or an alloy containing silver can be used, for example. An example of the alloy containing silver is an alloy of silver, palladium, and copper (APC). In the above manner, the display apparatus 100 can have high light extraction efficiency. Note that a metal other than silver may be used for the conductive layer 152b.

When the conductive layers 151 and 152 serve as the anode, a layer having a high work function is preferably used as the conductive layer 152c. The conductive layer 152c has a higher work function than the conductive layer 152b, for example. For the conductive layer 152c, a material similar to the material usable for the conductive layer 152a can be used, for example. For example, the conductive layers 152a and 152c can be formed using the same kind of material. For example, in the case where indium tin oxide is used for the conductive layer 152a, indium tin oxide can also be used for the conductive layer 152c.

When the conductive layers 151 and 152 serve as the cathode, the conductive layer 152c is preferably a layer having a low work function. The conductive layer 152c has a lower work function than the conductive layer 152b, for example.

The conductive layer 152c is preferably a layer having high visible light transmittance (e.g., transmittance with respect to light with a predetermined wavelength longer than or equal to 400 nm and shorter than 750 nm). For example, the visible light transmittance of the conductive layer 152c is preferably higher than that of the conductive layers 151 and 152b. The visible light transmittance of the conductive layer 152c can be, for example, higher than or equal to 60% and lower than or equal to 100%, and is preferably higher than or equal to 70% and lower than or equal to 100%, further preferably higher than or equal to 80% and lower than or equal to 100%. In that case, the amount of light that is absorbed by the conductive layer 152c after being emitted from the organic compound layer 103 can be reduced. As described above, the conductive layer 152b under the conductive layer 152c can be a layer having high visible light reflectance. Thus, the display apparatus 100 can have high light extraction efficiency.

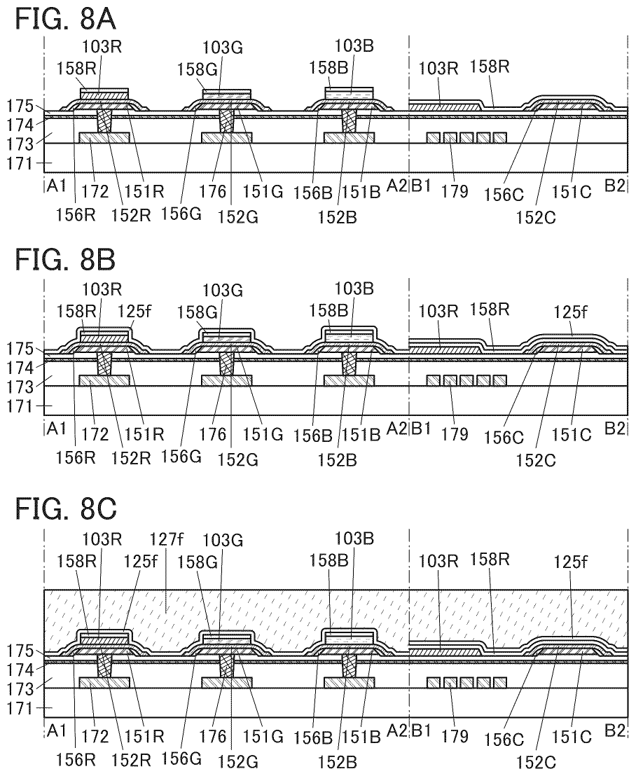

Next, an exemplary method for manufacturing the display apparatus 100 having the structure illustrated in FIG. 3A is described with reference to FIGS. 8A to 8C, FIGS. 9A to 9C, FIGS. 10A to 10C, FIGS. 11A and 11B, FIGS. 12A and 12B, and FIGS. 13A to 13D. An organic compound layer of the light-emitting device included in the display apparatus 100 is formed by manufacturing steps including a process using water. The use of the organic compound of one embodiment of the present invention for the organic compound layer of the light-emitting device included in the display apparatus of one embodiment of the present invention prevents problems such as dissolution of a layer containing the organic compound and permeation of a chemical solution into the layer even in the manufacture by a manufacturing method including a process using water; consequently, the light-emitting device can have favorable characteristics.

### Manufacturing Method Example

Thin films included in the display apparatus (e.g., insulating films, semiconductor films, and conductive films) can be formed by a sputtering method, a chemical vapor deposition (CVD) method, a vacuum evaporation method, a pulsed laser deposition (PLD) method, an ALD method, or the like. Examples of a CVD method include a plasma-enhanced CVD (PECVD) method and a thermal CVD method. An example of a thermal CVD method is a metal organic CVD (MOCVD) method.

Thin films included in the display apparatus (e.g., insulating films, semiconductor films, and conductive films) can also be formed by a wet film-formation method such as spin coating, dipping, spray coating, ink-jetting, dispensing, screen printing, offset printing, doctor blade coating, slit coating, roll coating, curtain coating, or knife coating.

Specifically, for fabrication of the light-emitting device, a vacuum process such as an evaporation method and a solution process such as a spin coating method or an ink-jet method can be used. Examples of an evaporation method include physical vapor deposition methods (PVD methods) such as a sputtering method, an ion plating method, an ion beam evaporation method, a molecular beam evaporation method, and a vacuum evaporation method, and a chemical vapor deposition method (CVD method). Specifically, the functional layers (e.g., the hole-injection layer, the hole-transport layer, the hole-blocking layer, the light-emitting layer, the electron-blocking layer, the electron-transport layer, and the electron-injection layer) included in the organic compound layer can be formed by an evaporation method (e.g., a vacuum evaporation method), a coating method (e.g., a dip coating method, a die coating method, a bar coating method, a spin coating method, or a spray coating method), a printing method (e.g., ink-jetting, screen printing (stencil), offset printing (planography), flexography (relief printing), gravure printing, or micro-contact printing), or the like.

Thin films included in the display apparatus can be processed by a lithography method, for example. Alternatively, a nanoimprinting method, a sandblasting method, a lift-off method, or the like may be used to process thin films. Alternatively, island-shaped thin films may be directly formed by a film formation method using a shielding mask such as a metal mask.

As a lithography method, for example, a photolithography method can be used. There are two typical examples of photolithography methods. In one of the methods, a resist mask is formed over a thin film that is to be processed, the thin film is processed by etching, for example, and then the resist mask is removed. In the other method, a photosensitive thin film is formed and then processed into a desired shape by light exposure and development.

As light used for exposure in the photolithography method, for example, light with an i-line (wavelength: 365 nm), light with a g-line (wavelength: 436 nm), light with an h-line (wavelength: 405 nm),or light in which the i-line, the g-line, and the h-line are mixed can be used. Alternatively, ultraviolet rays, KrF laser light, ArF laser light, or the like can be used. Exposure may be performed by liquid immersion exposure technique. As the light for exposure, extreme ultraviolet (EUV) light or X-rays may also be used. Furthermore, instead of the light used for exposure, an electron beam can be used. It is preferable to use EUV light, X-rays, or an electron beam to perform extremely minute processing. Note that when exposure is performed by scanning of a beam such as an electron beam, a photomask is not needed.

For etching of thin films, a dry etching method, a wet etching method, a sandblasting method, or the like can be used.

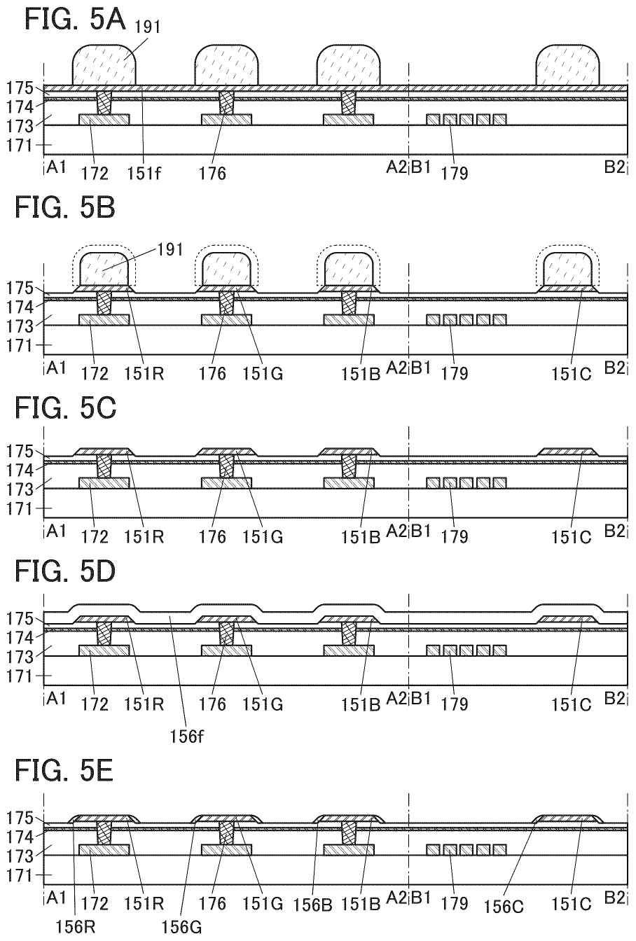

First, as illustrated in FIG. 5A, the insulating layer 171 is formed over a substrate (not illustrated). Next, the conductive layer 172 and a conductive layer 179 are formed over the insulating layer 171, and the insulating layer 173 is formed over the insulating layer 171 so as to cover the conductive layer 172 and the conductive layer 179. Then, the insulating layer 174 is formed over the insulating layer 173, and the insulating layer 175 is formed over the insulating layer 174.

As the substrate, a substrate having heat resistance high enough to withstand at least heat treatment performed later can be used. When an insulating substrate is used, it is possible to use a glass substrate, a quartz substrate, a sapphire substrate, a ceramic substrate, an organic resin substrate, or the like. Alternatively, it is possible to use a semiconductor substrate such as a single crystal semiconductor substrate or a polycrystalline semiconductor substrate of silicon, silicon carbide, or the like, a compound semiconductor substrate of silicon germanium or the like, or an SOT substrate.

Next, as illustrated in FIG. 5A, openings reaching the conductive layer 172 are formed in the insulating layers 175, 174, and 173. Then, the plugs 176 are formed to fill the openings.

Next, as illustrated in FIG. 5A, a conductive film 151f to be the conductive layers 151R, 151G, 151B, and 151C is formed over the plugs 176 and the insulating layer 175. The conductive film 151f can be formed by a sputtering method or a vacuum evaporation method, for example. A metal material can be used for the conductive film 151f, for example.

Subsequently, a resist mask 191 is formed over the conductive film 151f as illustrated in FIG. 5A. The resist mask 191 can be formed by application of a photosensitive material (photoresist), light exposure, and development.

Subsequently, as illustrated in FIG. 5B, the conductive film 151f in a region not overlapping with the resist mask 191, for example, is removed by an etching method, specifically, a dry etching method, for instance. Note that in the case where the conductive film 151f includes a layer formed using a conductive oxide such as indium tin oxide, for example, the layer may be removed by a wet etching method. In this manner, the conductive layer 151 is formed. In the case where part of the conductive film 151f is removed by a dry etching method, for example, a recessed portion may be formed in a region of the insulating layer 175 not overlapping with the conductive layer 151.

Next, the resist mask 191 is removed as illustrated in FIG. 5C. The resist mask 191 can be removed by ashing using oxygen plasma, for example. Alternatively, an oxygen gas and any of CF₄, C₄F₈, SF₆, CHF₃, Cl₂, H₂O, BCl₃, and a Group 18 element such as He may be used. Alternatively, the resist mask 191 may be removed by wet etching.

Then, as illustrated in FIG. 5D, an insulating film 156f to be an insulating layer 156R, an insulating layer 156G, an insulating layer 156B, and an insulating layer 156C is formed over the conductive layer 151R, the conductive layer 151G, the conductive layer 151B, the conductive layer 151C, and the insulating layer 175. The insulating film 156f can be formed by a CVD method, an ALD method, a sputtering method, or a vacuum evaporation method, for example.

For the insulating film 156f, an inorganic material can be used. As the insulating film 156f, an inorganic insulating film such as an oxide insulating film, a nitride insulating film, an oxynitride insulating film, or a nitride oxide insulating film can be used, for example. For example, an oxide insulating film including silicon, a nitride insulating film including silicon, an oxynitride insulating film including silicon, a nitride oxide insulating film including silicon, or the like can be used as the insulating film 156f. For the insulating film 156f, silicon oxynitride can be used, for example.

Subsequently, as illustrated in FIG. 5E, the insulating film 156f is processed to form the insulating layers 156R, 156G, 156B, and 156C. The insulating layer 156 can be formed by performing etching substantially uniformly on the top surface of the insulating film 156f, for example. Such uniform etching for planarization is also referred to as etch back treatment. Note that the insulating layer 156 may be formed by a lithography method.

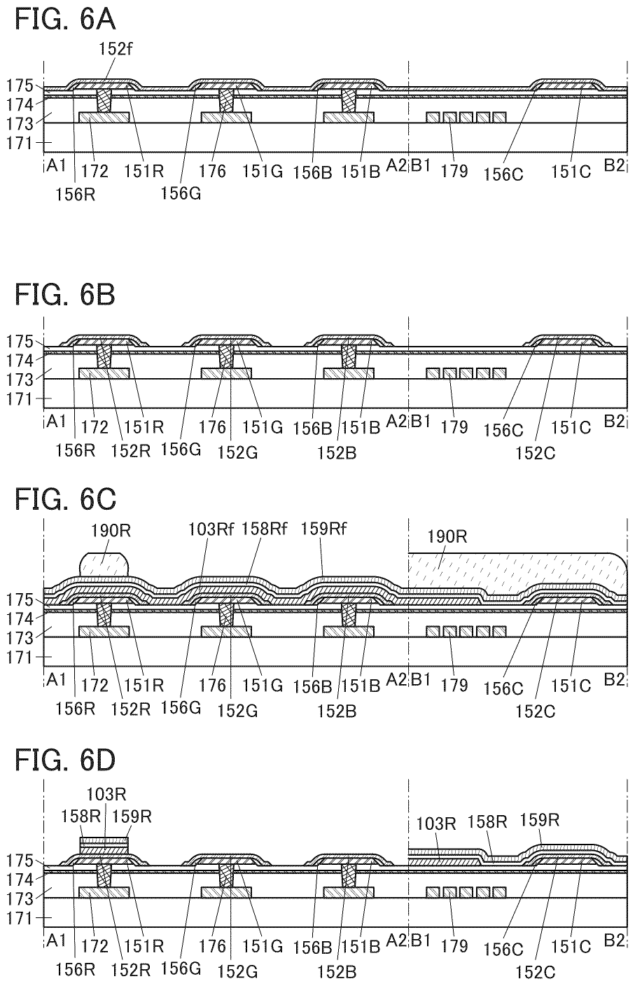

Then, as illustrated in FIG. 6A, a conductive film 152f to be the conductive layers 152R, 152G, and 152B and a conductive layer 152C is formed over the conductive layers 151R, 151G, 151n, and 151C and the insulating layers 156R, 156G, 156B, 156C, and 175. Specifically, the conductive film 152f is formed to cover the conductive layers 151R, 151G, 151B, and 151C and the insulating layers 156R, 156G, 156B, and 156C, for example.

The conductive film 152f can be formed by a sputtering method or a vacuum evaporation method, for example. A conductive oxide can be used for the conductive film 152f, for example. The conductive film 152f can be a stack of a film formed using a metal material and a film formed thereover using a conductive oxide. For example, the conductive film 152f can be a stack of a film formed using titanium, silver, or an alloy containing silver and a film formed thereover using a conductive oxide.

The conductive film 152f can be formed by an ALD method. In this case, for the conductive film 152f, an oxide containing one or more selected from indium, tin, zinc, gallium, titanium, aluminum, and silicon can be used. In this case, the conductive film 152f can be formed by repeating a cycle of introduction of a precursor (generally referred to as a metal precursor or the like in some cases), purge of the precursor, introduction of an oxidizer (generally referred to as a reactant, a non-metal precursor, or the like in some cases), and purge of the oxidizer. Here, in the case where an oxide film including a plurality of kinds of metals (e.g., an indium tin oxide film) is formed as the conductive film 152f, the composition of the metals can be controlled by varying the number of cycles for different kinds of precursors.

For example, in the case where an indium tin oxide film is formed as the conductive film 152f, after a precursor containing indium is introduced, the precursor is purged, and an oxidizer is introduced to form an In—O film, and then a precursor containing tin is introduced, the precursor is purged, and an oxidizer is introduced to form a Sn—O film. Here, when the number of cycles of forming an In—O film is larger than the number of cycles of forming a Sn—O film, the number of In atoms included in the conductive film 152f can be larger than the number of Sn atoms included therein.

For example, to form a zinc oxide film as the conductive film 152f, a Zn—O film is formed in the above procedure. For another example, to form an aluminum zinc oxide film as the conductive film 152f, a Zn—O film and an Al—O film are formed in the above procedure. For another example, to form a titanium oxide film as the conductive film 152f, a Ti—O film is formed in the above procedure. For another example, to form an indium tin oxide film including silicon as the conductive film 152f, an In—O film, a Sn—O film, and a Si—O film are formed in the above procedure. For another example, to form a zinc oxide film including gallium, a Ga—O film and a Zn—O film are formed in the above procedure.

As a precursor containing indium, it is possible to use, for example, triethylindium, trimethylindium, or [1,1,1-trimethyl-N-(trimethylsilyl)amide]-indium. As a precursor containing tin, it is possible to use, for example, tin chloride or tetrakis(dimethylamido)tin. As a precursor containing zinc, it is possible to use, for example, diethylzinc or dimethylzinc. As a precursor containing gallium, it is possible to use, for example, triethylgallium. As a precursor containing titanium, it is possible to use, for example, titanium chloride, tetrakis(dimethylamido)titanium, or tetraisopropyl titanate. As a precursor containing aluminum, it is possible to use, for example, aluminum chloride or trimethylaluminum. As a precursor containing silicon, it is possible to use, for example, trisilylamine, bis(diethylamino)silane, tris(dimethylamino)silane, bis(tert-butylamino)silane, or bis(ethylmethylamino)silane. As the oxidizer, water vapor, oxygen plasma, or an ozone gas can be used.

Then, as illustrated in FIG. 6B, the conductive film 152f is processed by a lithography method, for example, whereby the conductive layers 152R, 152G, 152B, and 152C are formed. Specifically, after a resist mask is formed, part of the conductive film 152f is removed by an etching method, for example. The conductive film 152f can be removed by a wet etching method, for example. The conductive film 152f may be removed by a dry etching method. Through the above steps, the pixel electrode including the conductive layer 151 and the conductive layer 152 is formed.

Next, hydrophobization treatment is preferably performed on the conductive layer 152. The hydrophobization treatment can change the hydrophilic properties of the subject surface to hydrophobic properties or increase the hydrophobic properties of the subject surface. The hydrophobization treatment for the conductive layer 152 can increase the adhesion between the conductive layer 152 and the organic compound layer 103 formed in a later step and inhibit film peeling. Note that the hydrophobization treatment is not necessarily performed.

Next, as illustrated in FIG. 6C, an organic compound film 103Rf to be the organic compound layer 103R is formed over the conductive layers 152R, 152G, and 152B and the insulating layer 175.

As illustrated in FIG. 6C, the organic compound film 103Rf is not formed over the conductive layer 152C. For example, a mask for defining a film formation area (also referred to as an area mask, a rough metal mask, or the like to be distinguished from a fine metal mask) is used, so that the organic compound film 103Rf can be formed only in a desired region. Employing a film formation step using an area mask and a processing step using a resist mask enables a light-emitting device to be fabricated by a relatively easy process.

The organic compound film 103Rf can be formed by an evaporation method, specifically a vacuum evaporation method, for example. The organic compound film 103Rf may be formed by a transfer method, a printing method, an ink-jet method, a coating method, or the like.

Next, as illustrated in FIG. 6C, a sacrificial film 158Rf to be a sacrificial layer 158R and a mask film 159Rf to be a mask layer 159R are sequentially formed over the organic compound film 103Rf, the conductive layer 152C, and the insulating layer 175. Note that in this specification and the like, a mask layer is sometimes referred to as a sacrificial layer.

Although this embodiment shows an example where a mask film having a two-layer structure of the sacrificial film 158Rf and the mask film 159Rf is formed, a mask film may have a single-layer structure or a stacked-layer structure of three or more layers.

Providing the sacrificial layer over the organic compound film 103Rf can reduce damage to the organic compound film 103Rf in the manufacturing process of the display apparatus, resulting in an increase in reliability of the light-emitting device.

As the sacrificial film 158Rf, a film that is highly resistant to the process conditions for the organic compound film 103Rf, specifically, a film having high etching selectivity with respect to the organic compound film 103Rf is used. For the mask film 159Rf, a film having high etching selectivity with respect to the sacrificial film 158Rf is used.

The sacrificial film 158Rf and the mask film 159Rf are formed at a temperature lower than the upper temperature limit of the organic compound film 103Rf. The typical substrate temperatures in formation of the sacrificial film 158Rf and the mask film 159Rf are each lower than or equal to 200° C., preferably lower than or equal to 150° C., further preferably lower than or equal to 120° C., still further preferably lower than or equal to 100° C., yet still further preferably lower than or equal to 80° C.

The sacrificial film 158Rf and the mask film 159Rf are preferably films that can be removed by a wet etching method. The use of a wet etching method can reduce damage to the organic compound film 103Rf in processing of the sacrificial film 158Rf and the mask film 159Rf, as compared to the case of using a dry etching method.

The sacrificial film 158Rf and the mask film 159Rf can be formed by a sputtering method, an ALD method (including a thermal ALD method and a PEALD method), a CVD method, or a vacuum evaporation method, for example. Alternatively, the sacrificial film 158Rf and the mask film 159Rf may be formed by the above-described wet film-formation method.

Note that the sacrificial film 158Rf that is formed over and in contact with the organic compound film 103Rf is preferably formed by a formation method that is less likely to damage the organic compound film 103Rf than a formation method of the mask film 159Rf. For example, the sacrificial film 158Rf is preferably formed by an ALD method or a vacuum evaporation method rather than a sputtering method.

As each of the sacrificial film 158Rf and the mask film 159Rf, one or more of a metal film, an alloy film, a metal oxide film, a semiconductor film, an organic insulating film, and an inorganic insulating film, for example, can be used.

For each of the sacrificial film 158Rf and the mask film 159Rf, it is possible to use a metal material such as gold, silver, platinum, magnesium, nickel, tungsten, chromium, molybdenum, iron, cobalt, copper, palladium, titanium, aluminum, yttrium, zirconium, or tantalum or an alloy material containing any of the metal materials, for example. It is particularly preferable to use a low-melting-point material such as aluminum or silver. A metal material that can block ultraviolet rays is preferably used for one or both of the sacrificial film 158Rf and the mask film 159Rf, in which case the organic compound film 103Rf can be inhibited from being irradiated with ultraviolet rays and deteriorating.

The sacrificial film 158Rf and the mask film 159Rf can each be formed using a metal oxide such as In—Ga—Zn oxide, indium oxide, In—Zn oxide, In—Sn oxide, indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or indium tin oxide containing silicon.

In place of gallium described above, an element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used.

As each of the sacrificial film and the mask film, a film including a material having a light-blocking property, particularly with respect to ultraviolet rays, is preferably used. Although a variety of materials such as a metal, an insulator, a semiconductor, and a metalloid that have a property of blocking ultraviolet rays can be used as a light-blocking material, each of the sacrificial film and the mask film is preferably a film capable of being processed by etching and is particularly preferably a film having good processability because part or the whole of each of the sacrificial film and the mask film is removed in a later step.

The sacrificial film and the mask film are preferably formed using a semiconductor material such as silicon or germanium, for example, for excellent compatibility with a semiconductor manufacturing process. Alternatively, an oxide or a nitride of the semiconductor material can be used. A non-metallic material such as carbon or a compound thereof can be used. A metal such as titanium, tantalum, tungsten, chromium, or aluminum or an alloy containing at least one of these metals can be used. Alternatively, an oxide containing the above-described metal, such as titanium oxide or chromium oxide, or a nitride such as titanium nitride, chromium nitride, or tantalum nitride can be used.

When a film including a material having a property of blocking ultraviolet rays is used as each of the sacrificial film and the mask film, the organic compound layer can be inhibited from being irradiated with ultraviolet rays in a light exposure step, for example. The organic compound layer is inhibited from being damaged by ultraviolet rays, so that the reliability of the light-emitting device can be improved.

Note that the same effect is obtained when a film including a material having a property of blocking ultraviolet rays is used for an after-mentioned inorganic insulating film 125f.

As each of the sacrificial film 158Rf and the mask film 159Rf, any of a variety of inorganic insulating films can be used. In particular, an oxide insulating film is preferable because its adhesion to the organic compound film 103Rf is higher than that of a nitride insulating film. For example, an inorganic insulating material such as aluminum oxide, hafnium oxide, or silicon oxide can be used for the sacrificial film 158Rf and the mask film 159Rf. As the sacrificial film 158Rf and the mask film 159Rf, aluminum oxide films can be formed by an ALD method, for example. An ALD method is preferably used, in which case damage to a base (in particular, the organic compound layer) can be reduced.

For example, an inorganic insulating film (e.g., an aluminum oxide film) formed by an ALD method can be used as the sacrificial film 158Rf, and an inorganic film (e.g., an In—Ga—Zn oxide film, an aluminum film, or a tungsten film) formed by a sputtering method can be used as the mask film 159Rf.

Note that the same inorganic insulating film can be used for both the sacrificial film 158Rf and the inorganic insulating layer 125 that is to be forned later. For example, an aluminum oxide film formed by an ALD method can be used for both the sacrificial film 158Rf and the inorganic insulating layer 125. For the sacrificial film 158Rf and the inorganic insulating layer 125, the same film formation conditions may be used or different film formation conditions may be used. For example, when the sacrificial film 158Rf is formed under conditions similar to those of the inorganic insulating layer 125, the sacrificial film 158Rf can be an insulating layer having a high barrier property against at least one of water and oxygen. Meanwhile, since the sacrificial film 158Rf is a layer a large part or the whole of which is to be removed in a later step, it is preferable that the processing of the sacrificial film 158Rf be easy. Therefore, the sacrificial film 158Rf is preferably formed with a substrate temperature lower than that for formation of the inorganic insulating layer 125.

One or both of the sacrificial film 158Rf and the mask film 159Rf may be formed using an organic material. For example, as the organic material, a material that can be dissolved in a solvent chemically stable with respect to at least the uppermost film of the organic compound film 103Rf may be used. Specifically, a material that will be dissolved in water or an alcohol can be suitably used. In forming a film of such a material, it is preferable to apply the material dissolved in a solvent such as water or an alcohol by a wet film-formation method and then perform heat treatment for evaporating the solvent. At this time, the heat treatment is preferably performed in a reduced-pressure atmosphere, in which case the solvent can be removed at a low temperature in a short time and thermal damage to the organic compound film 103Rf can be reduced accordingly.

The sacrificial film 158Rf and the mask film 159Rf may be formed using an organic resin such as polyvinyl alcohol (PVA), polyvinyl butyral, polyvinylpyrrolidone, polyethylene glycol, polyglycerin, pullulan, water-soluble cellulose, an alcohol-soluble polyamide resin, or a fluorine resin like perfluoropolymer.

For example, an organic film (e.g., a PVA film) formed by an evaporation method or any of the above wet film-formation methods can be used as the sacrificial film 158Rf, and an inorganic film (e.g., a silicon nitride film) formed by a sputtering method can be used as the mask film 159Rf.

Subsequently, a resist mask 190R is formed over the mask film 159Rf as illustrated in FIG. 6C. The resist mask 190R can be formed by application of a photosensitive material (photoresist), light exposure, and development.

The resist mask 190R may be formed using either a positive resist material or a negative resist material.

The resist mask 190R is provided at a position overlapping with the conductive layer 152R. The resist mask 190R is preferably provided also at a position overlapping with the conductive layer 152C. This can inhibit the conductive layer 152C from being damaged during the process of manufacturing the display apparatus. Note that the resist mask 190R is not necessarily provided over the conductive layer 152C. The resist mask 190R is preferably provided to cover the area from an end portion of the organic compound film 103Rf to an end portion of the conductive layer 152C (the end portion closer to the organic compound film 103Rf), as illustrated in the cross-sectional view along the line B1-B2 in FIG. 6C.

Next, as illustrated in FIG. 6D, part of the mask film 159Rf is removed using the resist mask 190R, so that the mask layer 159R is formed. The mask layer 159R remains over the conductive layers 152R and 152C. After that, the resist mask 190R is removed. Then, part of the sacrificial film 158Rf is removed using the mask layer 159R as a mask (also referred to as a hard mask), so that the sacrificial layer 158R is formed.

Each of the sacrificial film 158Rf and the mask film 159Rf can be processed by a wet etching method or a dry etching method. The sacrificial film 158Rf and the mask film 159Rf are preferably processed by isotropic etching.

The use of a wet etching method can reduce damage to the organic compound film 103Rf in processing of the sacrificial film 158Rf and the mask film 159Rf, as compared to the case of using a dry etching method. In the case of using a wet etching method, it is preferable to use a developer, an aqueous solution of tetramethylammonium hydroxide (TMAH), dilute hydrofluoric acid, oxalic acid, phosphoric acid, acetic acid, nitric acid, or a chemical solution containing a mixed solution of any of these acids, for example.

Since the organic compound film 103Rf is not exposed in the processing of the mask film 159Rf, the range of choice for a processing method for the mask film 159Rf is wider than that for the sacrificial film 158Rf. Specifically, even in the case where a gas containing oxygen is used as the etching gas in the processing of the mask film 159Rf, deterioration of the organic compound film 103Rf can be inhibited.

In the case of using a dry etching method to process the sacrificial film 158Rf, deterioration of the organic compound film 103Rf can be inhibited by not using a gas containing oxygen as the etching gas. In the case of using a dry etching method, it is preferable to use a gas containing CF₄, C₄F₈, SF₆, CHF₃, Cl₂, H₂O, BCl₃, or a Group 18 element such as He, for example, as the etching gas.

For example, in the case where an aluminum oxide film formed by an ALD method is used as the sacrificial film 158Rf, part of the sacrificial film 158Rf can be removed by a dry etching method using CHF₃ and lie or a combination of CHF₃, He, and CH₄. In the case where an In—Ga—Zn oxide film formed by a sputtering method is used as the mask film 159Rf, part of the mask film 159Rf can be removed by a wet etching method using diluted phosphoric acid. Alternatively, part of the mask film 159Rf may be removed by a dry etching method using CH₄ and Ar. Alternatively, part of the mask film 159Rf can be removed by a wet etching method using diluted phosphoric acid. In the case where a tungsten film formed by a sputtering method is used as the mask film 159Rf, part of the mask film 159Rf can be removed by a dry etching method using a combination of SF₆, CF₄, and O₂ or a combination of CF₄, Cl₂, and O₂.

The resist mask 190R can be removed by a method similar to that for the resist mask 191. For example, the resist mask 190R can be removed by ashing using oxygen plasma. Alternatively, an oxygen gas and any of CF₄, C₄F₈, SF₆, CHF₃, Cl₂, H₂O, BCl₃, and a Group 18 element such as He may be used. Alternatively, the resist mask 190R may be removed by wet etching. At this time, the sacrificial film 158Rf is located on the outermost surface, and the organic compound film 103Rf is not exposed; thus, the organic compound film 103Rf can be inhibited from being damaged in the step of removing the resist mask 190R. In addition, the range of choice for the method for removing the resist mask 190R can be widened.

Next, as illustrated in FIG. 6D, the organic compound film 103Rf is processed, so that the organic compound layer 103R is formed. For example, part of the organic compound film 103Rf is removed using the mask layer 159R and the sacrificial layer 158R as a hard mask to form the organic compound layer 103R.

Accordingly, as illustrated in FIG. 6D, the stacked-layer structure of the organic compound layer 103R, the sacrificial layer 158R, and the mask layer 159R remains over the conductive layer 152R. The conductive layers 152G and 152B are exposed.

In the example illustrated in FIG. 6D, an end portion of the organic compound layer 103R is located outward from an end portion of the conductive layer 152R. Such a structure can increase the aperture ratio of the pixel. Although not illustrated in FIG. 6D, by the above etching treatment, a recessed portion may be formed in the insulating layer 175 in a region not overlapping with the organic compound layer 103R.

Since the organic compound layer 103R covers the top surface and the side surface of the conductive layer 152R, the subsequent steps can be performed without exposure of the conductive layer 152R. If the end portion of the conductive layer 152R is exposed, there is a possibility that corrosion occurs in an etching step, for example. A product generated by corrosion of the conductive layer 152R may be unstable, and for example, might be dissolved in a solution when wet etching is performed and might be scattered in an atmosphere when dry etching is performed, By dissolution of the product in a solution or scattering of the product in the atmosphere, the product might be attached to a subject surface and the side surface of the organic compound layer 103R, for example, which might adversely affect the characteristics of the light-emitting device or form a leak path between a plurality of light-emitting devices. In a region where the end portion of the conductive layer 152R is exposed, adhesion between layers in contact with each other might be lowered, which might be likely to cause peeling of the organic compound layer 103R or the conductive layer 152R.

Accordingly, the structure where the organic compound layer 103R covers the top surface and the side surface of the conductive layer 152R can improve the yield and characteristics of the light-emitting device, for example.

As described above, the resist mask 190R is preferably provided to cover the area from the end portion of the organic compound layer 103R to the end portion of the conductive layer 152C (the end portion closer to the organic compound layer 103R) in the cross section along the dashed-dotted line B1-B2. In that case, as illustrated in FIG. 6D, the sacrificial layer 158R and the mask layer 159R are provided to cover the area from the end portion of the organic compound layer 103R to the end portion of the conductive layer 152C (the end portion closer to the organic compound layer 103R) in the cross section along the dashed-dotted line B1-B2. Hence, the insulating layer 175 can be inhibited from being exposed in the cross section along the dashed-dotted line B1-B2, for example. This can prevent the insulating layers 175, 174, and 173 from being partly removed by etching and thus prevent the conductive layer 179 from being exposed. Accordingly, the conductive layer 179 can be inhibited from being unintentionally electrically connected to another conductive layer. For example, a short circuit between the conductive layer 179 and a common electrode 155 formed in a later step can be inhibited.

The organic compound film 103Rf is preferably processed by anisotropic etching. Anisotropic dry etching is particularly preferable. Alternatively, wet etching may be used.

In the case of using a dry etching method, deterioration of the organic compound film 103Rf can be inhibited by not using a gas containing oxygen as the etching gas.

A gas containing oxygen may be used as the etching gas. When the etching gas contains oxygen, the etching rate can be increased. Therefore, the etching can be performed under a low-power condition while an adequately high etching rate is maintained. Accordingly, damage to the organic compound film 103Rf can be reduced. Furthermore, a defect such as attachment of a reaction product generated in the etching can be inhibited.

In the case of using a dry etching method, it is preferable to use a gas containing at least one of H₂, CF₄, C₄F₈, SF₆, CHF₃, Cl₂, H₂O, BCl₃, and a Group 18 element such as He and Ar as the etching gas, for example. Alternatively, a gas containing oxygen and at least one of the above is preferably used as the etching gas. Alternatively, an oxygen gas may be used as the etching gas. Specifically, for example, a gas containing H₂ and Ar or a gas containing CF₄ and He can be used as the etching gas. For another example, a gas containing CF₄, He, and oxygen can be used as the etching gas. For another example, a gas containing H₂ and Ar and a gas containing oxygen can be used as the etching gas.

As described above, in one embodiment of the present invention, the mask layer 159R is formed in the following manner: the resist mask 190R is formed over the mask film 159Rf and part of the mask film 159Rf is removed using the resist mask 190R. After that, part of the organic compound film 103Rf is removed using the mask layer 159R as a hard mask, so that the organic compound layer 103R is formed. In other words, the organic compound layer 103R is formed by processing the organic compound film 103Rf by a lithography method. Note that part of the organic compound film 103Rf may be removed using the resist mask 190R. Then, the resist mask 190R may be removed.

Next, hydrophobization treatment for the conductive layer 152G, for example, is preferably performed. At the time of processing the organic compound film 103Rf, the properties of a surface of the conductive layer 152G change to hydrophilic properties in some cases, for example. The hydrophobization treatment for the conductive layer 152G, for example, can increase the adhesion between the conductive layer 152G and a layer to be formed in a later step (which is the organic compound layer 103G here) and inhibit film peeling. Note that the hydrophobization treatment is not necessarily performed.

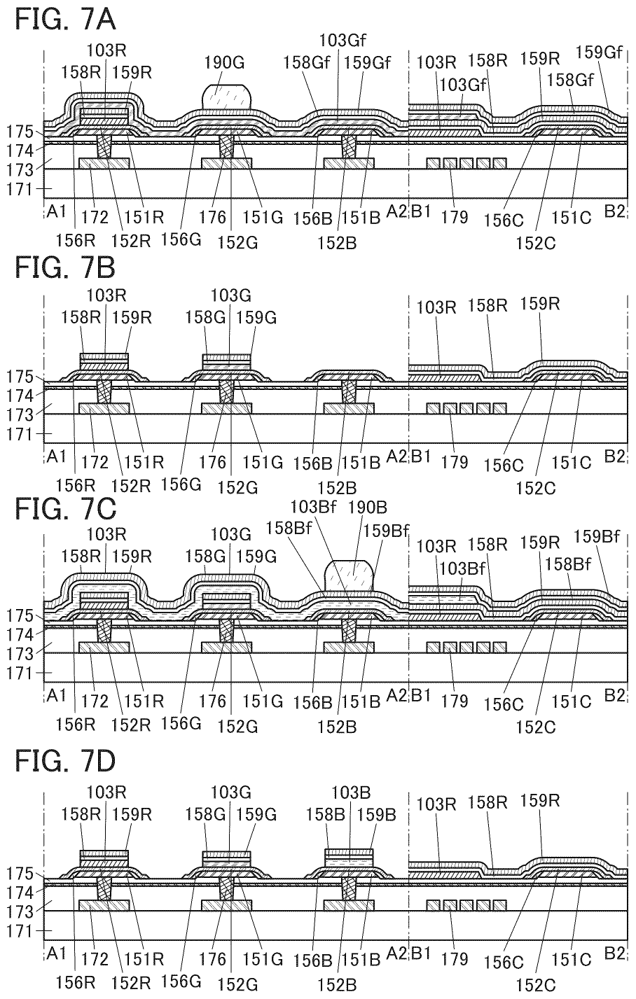

Next, as illustrated in FIG. 7A, an organic compound film 103Gf to be the organic compound layer 103G is formed over the conductive layer 152G, the conductive layer 152B, the mask layer 159R, and the insulating layer 175.

The organic compound film 103Gf can be formed by a method similar to that for forming the organic compound film 103Rf. The organic compound film 103Gf can have a structure similar to that of the organic compound film 103Rf.

Then, as illustrated in FIG. 7A, a sacrificial film 158Gf to be a sacrificial layer 158G and a mask film 159Gf to be a mask layer 159G are sequentially formed over the organic compound film 103Gf and the mask layer 159R. After that, a resist mask 190G is formed. The materials and the formation methods of the sacrificial film 158Gf and the mask film 159Gf are similar to those of the sacrificial film 158Rf and the mask film 159Rf. The material and the formation method of the resist mask 190G are similar to those of the resist mask 190R.

The resist mask 190G is provided at a position overlapping with the conductive layer 152G.

Subsequently, as illustrated in FIG. 7B, part of the mask film 159Gf is removed using the resist mask 190G, so that the mask layer 159G is formed. The mask layer 159G remains over the conductive layer 152G. After that, the resist mask 190G is removed. Then, part of the sacrificial film 158Gf is removed using the mask layer 159G as a mask, so that the sacrificial layer 158G is formed. Next, the organic compound film 103Gf is processed, so that the organic compound layer 103G is formed. For example, part of the organic compound film 103Gf is removed using the mask layer 159G and the sacrificial layer 158G as a hard mask to form the organic compound layer 103G.

Accordingly, as illustrated in FIG. 7B, the stacked-layer structure of the organic compound layer 103G, the sacrificial layer 158G, and the mask layer 159G remains over the conductive layer 152G. The mask layer 159R and the conductive layer 152B are exposed.

Next, hydrophobization treatment for the conductive layer 152B, for example, is preferably performed. At the time of processing the organic compound film 103Gf, the properties of a surface of the conductive layer 152B change to hydrophilic properties in some cases, for example. The hydrophobization treatment for the conductive layer 152B, for example, can increase the adhesion between the conductive layer 152B and a layer to be formed in a later step (which is the organic compound layer 103B here) and inhibit film peeling. Note that the hydrophobization treatment is not necessarily performed.

Next, as illustrated in FIG. 7C, an organic compound film 103Bf to be the organic compound layer 103B is formed over the conductive layer 152B, the mask layer 159R, the mask layer 159G, and the insulating layer 175.

The organic compound film 103Bf can be formed by a method similar to that for forming the organic compound film 103Rf. The organic compound film 103Bf can have a structure similar to that of the organic compound film 103Rf.

Then, as illustrated in FIG. 7C, a sacrificial film 158Bf to be a sacrificial layer 158B and a mask film 159Bf to be a mask layer 159B are sequentially formed over the organic compound film 103Bf and the mask layer 159R. After that, a resist mask 190B is formed. The materials and the formation methods of the sacrificial film 158Bf and the mask film 159Bf are similar to those of the sacrificial film 158Rf and the mask film 159Rf. The material and the formation method of the resist mask 190B are similar to those of the resist mask 190R.

The resist mask 190B is provided at a position overlapping with the conductive layer 152B.

Subsequently, as illustrated in FIG. 7D, part of the mask film 159Bf is removed using the resist mask 190B, so that the mask layer 159B is formed. The mask layer 159B remains over the conductive layer 152B. After that, the resist mask 190B is removed. Then, part of the sacrificial film 158Bf is removed using the mask layer 159B as a mask, so that the sacrificial layer 158B is formed. Next, the organic compound film l03Bf is processed, so that the organic compound layer 103B is formed. For example, part of the organic compound film 103Bf is removed using the mask layer 159B and the sacrificial layer 158B as a hard mask to form the organic compound layer 103B.

Accordingly, as illustrated in FIG. 7D, the stacked-layer structure of the organic compound layer 103B, the sacrificial layer 158B, and the mask layer 159B remains over the conductive layer 152B. The mask layers 159R and 159G are exposed.

Note that the side surfaces of the organic compound layers 103R, 103G, and 103B are preferably perpendicular or substantially perpendicular to their formation surfaces. For example, the angle between the formation surfaces and these side surfaces is preferably greater than or equal to 60° and less than or equal to 90°.

The distance between two adjacent layers among the organic compound layers 103R, 103G, and 103B, which are formed by a lithography method as described above, can be shortened to less than or equal to 8 μm, less than or equal to 5 μm, less than or equal to 3 μm, less than or equal to 2 μm, or less than or equal to 1 μm. Here, the distance can be specified, for example, by the distance between opposite end portions of two adjacent layers among the organic compound layers 103R, 103G, and 103B. Shortening the distance between the island-shaped organic compound layers can provide a display apparatus having high resolution and a high aperture ratio. In addition, the distance between the first electrodes of adjacent light-emitting devices can also be shortened to be, for example, less than or equal to 10 μm, less than or equal to 8 μm, less than or equal to 5 μm, less than or equal to 3 μm, or less than or equal to 2 μm. Note that the distance between the first electrodes of adjacent light-emitting devices is preferably greater than or equal to 2 μm and less than or equal to 5 μm.

Next, as illustrated in FIG. 8A, the mask layers 159R, 159G, and 159B are preferably removed. The sacrificial layers 158R, 158G, and 158B and the mask layers 159R, 159G, and 159B remain in the display apparatus in some cases depending on the subsequent steps. Removing the mask layers 159R, 159G, and 159B at this stage can inhibit the mask layers 159R, 159G, and 159B from being left in the display apparatus. For example, in the case where a conductive material is used for the mask layers 159R, 159G, and 159B, removing the mask layers 159R, 159G, and 159B in advance can inhibit generation of a leakage current, formation of a capacitor, and the like due to the remaining mask layers 159R, 159G, and 159B.

This embodiment describes an example where the mask layers 159R, 159G, and 159B are removed; however, the mask layers 159R, 159G, and 159B are not necessarily removed. For example, in the case where the mask layers 159R, 159G, and 159B include the above-described material having a property of blocking ultraviolet rays, the procedure preferably proceeds to the next step without removing the mask layers 159R, 159G, and 159B, in which case the organic compound layers can be protected from ultraviolet rays.

The step of removing the mask layers can be performed by a method similar to that for the step of processing the mask films. Specifically, by using a wet etching method, damage applied to the organic compound layers 103R, 103G, and 103B at the time of removing the mask layers can be reduced as compared to the case of using a dry etching method.

The mask layers may be removed by being dissolved in a solvent such as water or an alcohol. Examples of an alcohol include ethyl alcohol, methyl alcohol, isopropyl alcohol (IPA), and glycerin.

After the mask layers are removed, drying treatment may be performed in order to remove water included in the organic compound layers 103R, 103G, and 103B and water adsorbed onto the surfaces of the organic compound layers 103R, 103G, and 103B. For example, heat treatment in an inert gas atmosphere or a reduced-pressure atmosphere can be performed. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 120° C. The heat treatment is preferably performed in a reduced-pressure atmosphere, in which case drying at a lower temperature is possible.

Next, as illustrated in FIG. 8B, the inorganic insulating film 125f to be the inorganic insulating layer 125 is formed to cover the organic compound layers 103R, 103G, and 103B and the sacrificial layers 158R, 158G, and 158B.

As described later, an insulating film to be the insulating layer 127 is formed in contact with the top surface of the inorganic insulating film 125f. Therefore, the top surface of the inorganic insulating film 125f preferably has a high affinity for the material used for the insulating film (e.g., a photosensitive resin composition containing an acrylic resin). To improve the affinity, surface treatment is preferably performed so that the top surface of the inorganic insulating film 125f is made hydrophobic or its hydrophobic properties are improved. For example, it is preferable to perform the treatment using a silylation agent such as hexamethyldisilazane (HMDS). By making the top surface of the inorganic insulating film 125f hydrophobic in such a manner, an insulating film 127f can be formed with favorable adhesion. Note that the above-described hydrophobization treatment may be performed as the surface treatment.

Then, as illustrated in FIG. 8C, the insulating film 127f to be the insulating layer 127 is formed over the inorganic insulating film 125f.

The inorganic insulating film 125f and the insulating film 127f are preferably formed by a formation method by which the organic compound layers 103R, 103G, and 103B are less damaged. The inorganic insulating film 125f, which is formed in contact with the side surfaces of the organic compound layers 103R, 103G, and 10313, is particularly preferably formed by a formation method that causes less damage to the organic compound layers 103R, 103G, and 103B than the formation method of the insulating film 127f.

Each of the inorganic insulating film 125f and the insulating film 127f is formed at a temperature lower than the upper temperature limits of the organic compound layers 103R, 103G, and 103B. When the inorganic insulating film 125f is formed at a high substrate temperature, the formed inorganic insulating film 125f, even with a small thickness, can have a low impurity concentration and a high barrier property against at least one of water and oxygen.

The substrate temperature at the time of forming the inorganic insulating film 125f and the insulating film 127f is preferably higher than or equal to 60° C., higher than or equal to 80° C., higher than or equal to 100° C., or higher than or equal to 120° C. and lower than or equal to 200° C., lower than or equal to 180° C., lower than or equal to 160° C., lower than or equal to 150° C., or lower than or equal to 140° C.

As the inorganic insulating film 125f, an insulating film having a thickness greater than or equal to 3 nm, greater than or equal to 5 nm, or greater than or equal to 10 nm and less than or equal to 200 nm, less than or equal to 150 nm, less than or equal to 100 nm, or less than or equal to 50 nm is preferably formed in the above-described range of the substrate temperature.

The inorganic insulating film 125f is preferably formed by an ALD method, for example. An ALD method is preferably used, in which case damage due to film formation is reduced and a film with good coverage can be formed. As the inorganic insulating film 125f, an aluminum oxide film is preferably formed by an ALD method, for example.

Alternatively, the inorganic insulating film 125f may be formed by a sputtering method, a CVD method, or a PECVD method, each of which has a higher film formation rate than an ALD method. In that case, a highly reliable display apparatus can be manufactured with high productivity.

The insulating film 127f is preferably formed by the aforementioned wet film-formation method. The insulating film 127f is preferably formed by spin coating using a photosensitive material, for example, and specifically preferably formed using a photosensitive resin composition containing an acrylic resin.

The insulating film 127f is preferably formed using a resin composition containing a polymer, an acid-generating agent, and a solvent, for example. The polymer is formed using one or more kinds of monomers and has a structure where one or more kinds of structural units (also referred to as building blocks) are repeated regularly or irregularly. As the acid-generating agent, one or both of a compound that generates an acid by light irradiation and a compound that generates an acid by heating can be used. The resin composition may also include one or more of a photosensitizing agent, a sensitizer, a catalyst, an adhesive aid, a surface-active agent, and an antioxidant.

Heat treatment (also referred to as prebaking) is preferably performed after the insulating film 127f is formed. The heat treatment is performed at a temperature lower than the upper temperature limits of the organic compound layers 103R, 103G, and 103B. The substrate temperature in the heat treatment is preferably higher than or equal to 50° C. and lower than or equal to 200° C., further preferably higher than or equal to 60° C. and lower than or equal to 150° C., still further preferably higher than or equal to 70° C. and lower than or equal to 120° C. Accordingly, the solvent included in the insulating film 127f can be removed.

Then, part of the insulating film 127f is exposed to visible light or ultraviolet rays. Here, when a positive photosensitive resin composition containing an acrylic resin is used for the insulating film 127f, a region where the insulating layer 127 is not formed in a later step is irradiated with visible light or ultraviolet rays. The insulating layer 127 is formed in regions that are sandwiched between any two of the conductive layers 152R, 152G, and 152B and around the conductive layer 152C. Thus, the top surfaces of the conductive layers 152R, 152G, 152B, and 152C are irradiated with visible light or ultraviolet rays. Note that when a negative photosensitive material is used for the insulating film 127f, the region where the insulating layer 127 is to be formed is irradiated with visible light or ultraviolet rays.

The width of the insulating layer 127 that is to be formed later can be controlled in accordance with the exposed region of the insulating film 127f. In this embodiment, processing is performed such that the insulating layer 127 includes a portion overlapping with the top surface of the conductive layer 151.

The light used for the exposure preferably includes the i-line (wavelength: 365 nm). Furthermore, the light used for the exposure may include at least one of the g-line (wavelength: 436 nm) and the h-line (wavelength: 405 nm).

Here, when a barrier insulating layer against oxygen (e.g., an aluminum oxide film) is provided as one or both of the sacrificial layer 158 (the sacrificial layers 158R, 158G, and 158B) and the inorganic insulating film 125f, diffusion of oxygen into the organic compound layers 103R, 103G, and 103B can be inhibited. When the organic compound layer is irradiated with light (visible light or ultraviolet rays), the organic compound included in the organic compound layer is brought into an excited state and a reaction between the organic compound and oxygen in the atmosphere is promoted in some cases. Specifically, when the organic compound layer is irradiated with light (visible light or ultraviolet rays) in an atmosphere containing oxygen, oxygen might be bonded to the organic compound included in the organic compound layer. By providing the sacrificial layer 158 and the inorganic insulating film 125f over the island-shaped organic compound layer, bonding of oxygen in the atmosphere to the organic compound included in the organic compound layer can be inhibited.

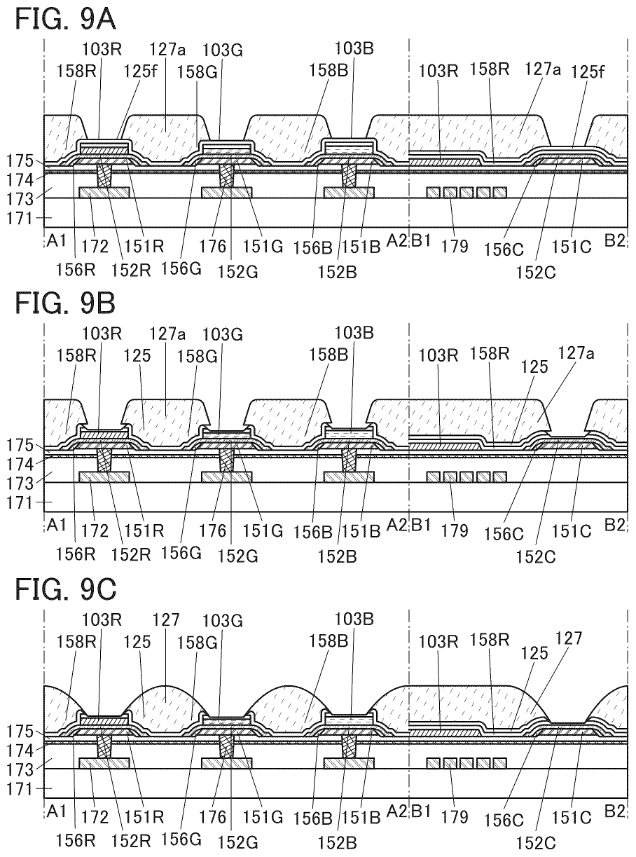

Next, as illustrated in FIG. 9A, development is performed to remove the exposed region of the insulating film 127f, so that an insulating layer 127a is formed. The insulating layer 127a is formed in regions that are sandwiched between any two of the conductive layers 152R, 152G, and 152B and a region surrounding the conductive layer 152C. Here, when an acrylic resin is used for the insulating film 127f, an alkaline solution, such as TMAH, can be used as a developer.

Then, a residue (scum) due to the development may be removed. For example, the residue can be removed by ashing using oxygen plasma.

Etching may be performed to adjust the surface level of the insulating layer 127a. The insulating layer 127a may be processed by ashing using oxygen plasma, for example. In the case where a non-photosensitive material is used for the insulating film 127f, the surface level of the insulating film 127f can be adjusted by the ashing, for example.

Next, as illustrated in FIG. 9B, etching treatment is performed using the insulating layer 127a as a mask to remove part of the inorganic insulating film 125f and reduce the thickness of part of the sacrificial layers 158R, 158G, and 158B. Thus, the inorganic insulating layer 125 is formed under the insulating layer 127a. Moreover, the surfaces of the thin portions in the sacrificial layers 158R, 158G, and 158B are exposed. Note that the etching treatment using the insulating layer 127a as a mask may be hereinafter referred to as first etching treatment.

The first etching treatment can be performed by dry etching or wet etching. Note that the inorganic insulating film 125f is preferably formed using a material similar to that of the sacrificial layers 158R, 158G, and 158B, in which case the first etching treatment can be performed collectively.

By etching using the insulating layer 127a with a tapered side surface as a mask, the side surface of the inorganic insulating layer 125 and upper end portions of the side surfaces of the sacrificial layers 158R, 158G, and 158B can be made to have a tapered shape relatively easily.

In the case of performing dry etching, a chlorine-based gas is preferably used. As the chlorine-based gas, one of Cl₂, BCl₃, SiCl₄, CCl₄, and the like or a mixture of two or more of them can be used. Moreover, one of an oxygen gas, a hydrogen gas, a helium gas, an argon gas, and the like or a mixture of two or more of them can be added as appropriate to the chlorine-based gas. By the dry etching, the thin regions of the sacrificial layers 158R, 158G, and 158B can be formed with favorable in-plane uniformity.

As a dry etching apparatus, a dry etching apparatus including a high-density plasma source can be used. As the dry etching apparatus including a high-density plasma source, an inductively coupled plasma (ICP) etching apparatus can be used, for example. Alternatively, a capacitively coupled plasma (CCP) etching apparatus including parallel plate electrodes can be used. The capacitively coupled plasma etching apparatus including parallel plate electrodes may have a structure where a high-frequency voltage is applied to one of the parallel plate electrodes. Alternatively, the capacitively coupled plasma etching apparatus may have a structure where different high-frequency voltages are applied to one of the parallel-plate electrodes. Alternatively, the capacitively coupled plasma etching apparatus may have a structure where high-frequency voltages with the same frequency are applied to the parallel-plate electrodes. Alternatively, the capacitively coupled plasma etching apparatus may have a structure where high-frequency voltages with different frequencies are applied to the parallel-plate electrodes.

In the case of performing dry etching, a by-product or the like generated by the dry etching might be deposited on the top surface and the side surface of the insulating layer 127a, for example. Accordingly, a component of the etching gas, a component of the inorganic insulating film 125f, a component of the sacrificial layers 158R, 158G, and 158B, and the like might be included in the insulating layer 127 in the completed display apparatus.

The first etching treatment is preferably performed by wet etching. The use of a wet etching method can reduce damage to the organic compound layers 103R, 103G, and 103B, as compared to the case of using a dry etching method. For example, the wet etching can be performed using an alkaline solution. For instance, TMAH, which is an alkaline solution, can be used for the wet etching of an aluminum oxide film. In this case, puddle wet etching can be performed. Note that the inorganic insulating film 125f is preferably formed using a material similar to that of the sacrificial layers 158R, 158G, and 158B, in which case the above etching treatment can be performed collectively.

The sacrificial layers 158R, 158G, and 158B are not removed completely by the first etching treatment, and the etching treatment is stopped when the thicknesses of the sacrificial layers 158R, 158G, and 158B are reduced. The sacrificial layers 158R, 158G, and 158B remain over the corresponding organic compound layers 103R, 103G, and 103B in this manner, whereby the organic compound layers 103R, 103G, and 103B can be prevented from being damaged by treatment in a later step.

Next, the insulating layer 127a is preferably irradiated with visible light or ultraviolet rays by performing light exposure on the entire substrate. The energy density for the light exposure is preferably greater than 0 mJ/cm² and less than or equal to 800 mJ/cm², further preferably greater than 0 mJ/cm² and less than or equal to 500 mJ/cm². Performing such light exposure after the development can sometimes increase the degree of transparency of the insulating layer 127a. In addition, it is sometimes possible to lower the substrate temperature required for subsequent heat treatment for changing the shape of the insulating layer 127a to a tapered shape.

Here, when a barrier insulating layer against oxygen (e.g., an aluminum oxide film) exists as each of the sacrificial layers 158R, 158G, and 158B, diffusion of oxygen into the organic compound layers 103R, 103G, and 103B can be inhibited. When the organic compound layer is irradiated with light (visible light or ultraviolet rays), the organic compound included in the organic compound layer is brought into an excited state and a reaction between the organic compound and oxygen in the atmosphere is promoted in some cases. Specifically, when the organic compound layer is irradiated with light (visible light or ultraviolet rays) in an atmosphere containing oxygen, oxygen might be bonded to the organic compound included in the organic compound layer. By providing the sacrificial layers 158R, 158G, and 158B over the island-shaped organic compound layers, bonding of oxygen in the atmosphere to the organic compounds included in the organic compound layers can be inhibited.

Then, heat treatment (also referred to as post-baking) is performed. The heat treatment can change the insulating layer 127a into the insulating layer 127 having a tapered side surface (FIG. 9C). The heat treatment is performed at a temperature lower than the upper temperature limit of the organic compound layer. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 130° C. The heating atmosphere may be an air atmosphere or an inert gas atmosphere. Moreover, the heating atmosphere may be an atmospheric-pressure atmosphere or a reduced-pressure atmosphere. The substrate temperature in the heat treatment of this step is preferably higher than that in the heat treatment (prebaking) after the formation of the insulating film 127f. In that case, adhesion between the insulating layer 127 and the inorganic insulating layer 125 can be improved, and corrosion resistance of the insulating layer 127 can be increased.

When the sacrificial layers 158R, 158G, and 158B are not completely removed by the first etching treatment and the thinned sacrificial layers 158R, 158G, and 158B are left, the organic compound layers 103R, 103G, and 103B can be prevented from being damaged and deteriorating in the heat treatment. This increases the reliability of the light-emitting devices.

Note that the side surface of the insulating layer 127 may have a concave shape depending on the material of the insulating layer 127 and the temperature, time, and atmosphere of the post-baking. For example, when the temperature of the post-baking is higher or the duration of the post-baking is longer, the insulating layer 127 is more likely to change in shape and thus a concave shape may be more likely to be formed.

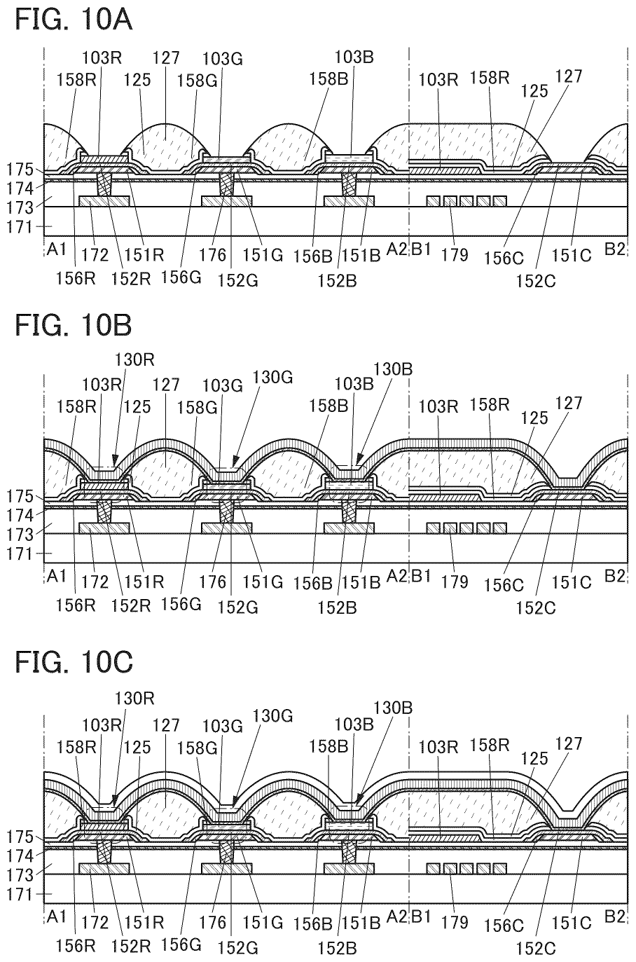

Next, as illustrated in FIG. 10A, etching treatment is performed using the insulating layer 127 as a mask to remove part of the sacrificial layers 158R, 158G, and 158B. Note that part of the inorganic insulating layer 125 is also removed in some cases. Thus, openings are formed in the sacrificial layers 158R, 158G, and 158B, and the top surfaces of the organic compound layers 103R, 103G, and 103B and the conductive layer 152C are exposed. Note that the etching treatment using the insulating layer 127 as a mask may be hereinafter referred to as second etching treatment.

An end portion of the inorganic insulating layer 125 is covered with the insulating layer 127. FIG. 10A illustrates an example where part of an end portion of the sacrificial layer 158G (specifically a tapered portion formed by the first etching treatment) is covered with the insulating layer 127 and a tapered portion formed by the second etching treatment is exposed.

If the first etching treatment is not performed and the inorganic insulating layer 125 and the mask layer are collectively etched after the post-baking, the inorganic insulating layer 125 and the mask layer under an end portion of the insulating layer 127 may disappear because of side etching and a void may be formed. The void causes unevenness on the formation surface of the common electrode 155, so that step disconnection is more likely to be caused in the common electrode 155. Even when a void is formed owing to side etching of the inorganic insulating layer 125 and the mask layer by the first etching treatment, the post-baking performed subsequently can make the insulating layer 127 fill the void. After that, the thinned mask layer is etched by the second etching treatment; thus, the amount of side etching decreases, a void is less likely to be formed, and even if a void is formed, it can be extremely small. Consequently, the formation surface of the common electrode 155 can be made flatter.

Note that the insulating layer 127 may cover the entire end portion of the sacrificial layer 158G. For example, the end portion of the insulating layer 127 may droop to cover the end portion of the sacrificial layer 158G. For another example, the end portion of the insulating layer 127 may be in contact with the top surface of at least one of the organic compound layers 103R, 103G, and 103B. As described above, when light exposure is not performed on the insulating layer 127a after the development, the shape of the insulating layer 127 may be likely to change.

The second etching treatment is performed by wet etching. The use of a wet etching method can reduce damage to the organic compound layers 103R, 103G, and 103B, as compared to the case of using a dry etching method. The wet etching can be performed using an alkaline solution such as TMAH, for example.

Meanwhile, in the case where the second etching treatment is performed by a wet etching method and gaps due to, for example, poor adhesion between the organic compound layer 103 and another layer exist at the interface between the organic compound layer 103 and the sacrificial layer 158, the interface between the organic compound layer 103 and the inorganic insulating layer 125, and the interface between the organic compound layer 103 and the insulating layer 175, the chemical solution used in the second etching treatment sometimes enters the gaps to come into contact with the pixel electrode. Here, when the chemical solution comes into contact with both the conductive layer 151 and the conductive layer 152, one of the conductive layers 151 and 152 that has a lower spontaneous potential than the other suffers from galvanic corrosion in some cases. For example, when the conductive layer 151 is formed using aluminum and the conductive layer 152 is formed using indium tin oxide, the conductive layer 152 sometimes corrodes. As a result, the yield of the display apparatus decreases in some cases. Moreover, the reliability of the display apparatus decreases in some cases.

The conductive layer 152, which covers the top and side surfaces of the conductive layer 151 as described above, can prevent the chemical solution from coming into contact with the conductive layer 151 in the second etching treatment even when gaps exist at the interface between the organic compound layer 103 and the sacrificial layer 158, the interface between the organic compound layer 103 and the inorganic insulating layer 125, and the interface between the organic compound layer 103 and the insulating layer 175. Thus, corrosion of the pixel electrode, e.g., the conductive layer 152, can be prevented.

Furthermore, when the insulating layer 156 is formed to include a region overlapping with the side surface of the conductive layer 151 and the conductive layer 152 is formed to cover the conductive layer 151 and the insulating layer 156, the step disconnection can be prevented, whereby the chemical solution can be prevented from coming into contact with the conductive layer 151 in the second etching treatment, for example. Thus, corrosion of the pixel electrode, e.g., the conductive layer 152, can be prevented.

As described above, by providing the insulating layer 127, the inorganic insulating layer 125, and the sacrificial layers 158R, 158G, and 158B, poor connection due to a disconnected portion and an increase in electrical resistance due to a locally thinned portion can be inhibited from occurring in the common electrode 155 between the light-emitting devices. Thus, the display apparatus of one embodiment of the present invention can have improved display quality.

Heat treatment is performed after the organic compound layers 103R, 103G, and 103B are partly exposed. By the heat treatment, water included in the organic compound layers and water adsorbed onto the surfaces of the organic compound layers, for example, can be removed. The shape of the insulating layer 127 may be changed by the heat treatment. Specifically, the insulating layer 127 may be widened to cover at least one of the end portion of the inorganic insulating layer 125, the end portions of the sacrificial layers 158R, 158G, and 158B, and the top surfaces of the organic compound layers 103R, 103G, and 103B.

If the temperature of the heat treatment is too low, water included in the organic compound layers and water adsorbed onto the surfaces of the organic compound layers, for example, cannot be sufficiently removed. If the temperature of the heat treatment is too high, the organic compound layer 103 might deteriorate and the insulating layer 127 might change in shape excessively. Therefore, the temperature of the heat treatment is preferably higher than the temperature at which water is released from the organic compound layer 103 and lower than T_{g} of the organic compound included in the organic compound layer 103, further preferably lower than T_{g} of the organic compound included in the upper surface of the organic compound layer 103. Specifically, the substrate temperature is preferably higher than or equal to 80° C. and lower than or equal to 130° C., further preferably higher than or equal to 90° C. and lower than or equal to 120° C., still further preferably higher than or equal to 100° C. and lower than or equal to 120° C., yet still further preferably higher than or equal to 100° C. and lower than or equal to 110° C. The heating atmosphere may be an air atmosphere or an inert gas atmosphere. Although the heating atmosphere may be an atmospheric-pressure atmosphere or a reduced-pressure atmosphere, a reduced-pressure atmosphere is preferably employed to prevent re-adsorption of water released from the organic compound layer 103.

By the heat treatment, water included in the organic compound layers and water adsorbed onto the surfaces of the organic compound layers, for example, can be sufficiently removed without deterioration of the organic compound layers 103R, 103G, and 103B and an excessive change in the shape of the insulating layer 127. Thus, degradation of the characteristics of the light-emitting device can be prevented.

Next, as illustrated in FIG. 10B, the common layer 104 and the common electrode 155 are formed over the organic compound layers 103R, 103G, and 103B, the conductive layer 152C, and the insulating layer 127. The common layer 104 and common electrode 155 can be formed by a sputtering method, a vacuum evaporation method, or the like. The common layer 104 may be formed by an evaporation method while the common electrode 155 may be formed by a sputtering method.

Next, as illustrated in FIG. 10C, the protective layer 131 is formed over the common electrode 155. The protective layer 131 can be formed by a vacuum evaporation method, a sputtering method, a CVD method, an ALD method, or the like.

Then, the substrate 120 is attached to the protective layer 131 using the resin layer 122, so that the display apparatus can be manufactured. In the method for manufacturing the display apparatus of one embodiment of the present invention, the insulating layer 156 is formed to include a region overlapping with the side surface of the conductive layer 151 and the conductive layer 152 is formed to cover the conductive layer 151 and the insulating layer 156 as described above. This can increase the yield of the display apparatus and inhibit generation of defects.

As described above, in the method for manufacturing the display apparatus of one embodiment of the present invention, the island-shaped organic compound layers 103R, 103G, and 103B are each formed not by using a fine metal mask but by processing a film formed on the entire surface; thus, the island-shaped layers can be formed to have a uniform thickness. Consequently, a high-resolution display apparatus or a display apparatus with a high aperture ratio can be obtained. Furthermore, even when the resolution or the aperture ratio is high and the distance between the subpixels is extremely short, the organic compound layers 103R, 103G, and 103B can be inhibited from being in contact with each other in the adjacent subpixels. As a result, generation of a leakage current between the subpixels can be inhibited. This can prevent crosstalk, so that a display apparatus with extremely high contrast can be obtained. Moreover, even a display apparatus that includes tandem light-emitting devices formed by a lithography method can have favorable characteristics.

The structure described in this embodiment can be used in combination with any of the structures described in other embodiments as appropriate.

### Embodiment 5

In this embodiment, a display apparatus of one embodiment of the present invention will be described.

The display apparatus in this embodiment can be a high-resolution display apparatus. Thus, the display apparatus in this embodiment can be used for display portions of information terminals (wearable devices) such as watch-type and bracelet-type information terminals and display portions of wearable devices capable of being worn on a head, such as a VR device like a head mounted display (HMD) and a glasses-type AR device.

The display apparatus in this embodiment can be a high-definition display apparatus or a large-sized display apparatus. Accordingly, the display apparatus in this embodiment can be used for display portions of a digital camera, a digital video camera, a digital photo frame, a mobile phone, a portable game console, a portable information terminal, and an audio reproducing device, in addition to display portions of electronic appliances with a relatively large screen, such as a television device, desktop and notebook personal computers, a monitor of a computer and the like, digital signage, and a large game machine such as a pachinko machine.

### [Display Module]

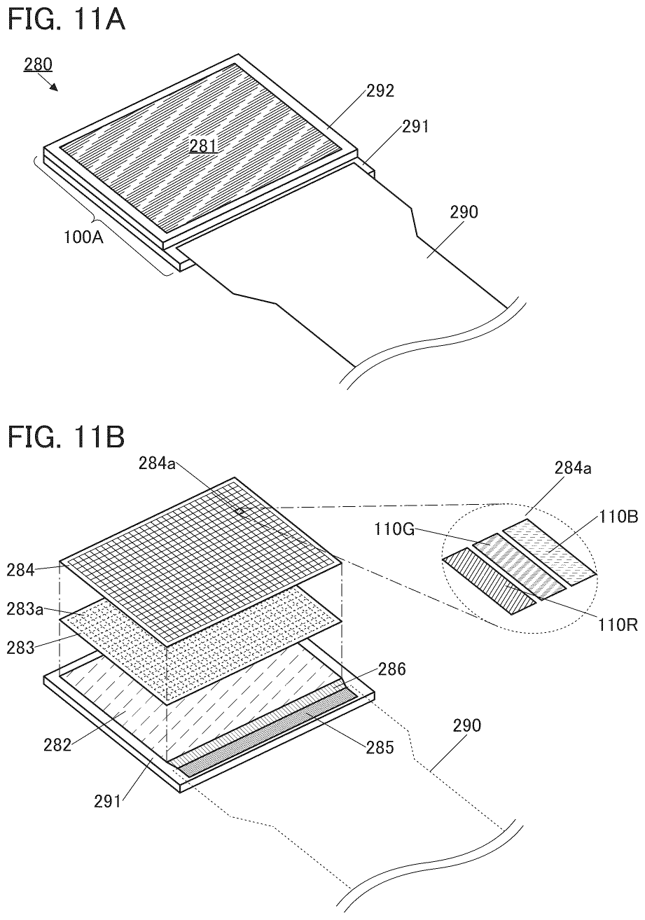

FIG. 11A is a perspective view of a display module 280. The display module 280 includes a display apparatus 100A and an FPC 290.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 11B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion not overlapping with the pixel portion 284 over the substrate 291. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 11B. The pixels 284a can employ any of the structures described in the above embodiments. FIG. 11B illustrates an example where the pixel 284a has a structure similar to that of the pixel 178 illustrated in FIGS. 3A and 3B.

The pixel circuit portion 283 includes a plurality of pixel circuits 283a arranged periodically.

One pixel circuit 283a is a circuit that controls driving of a plurality of elements included in one pixel 284a. One pixel circuit 283a can be provided with three circuits each of which controls light emission of one light-emitting device. For example, the pixel circuit 283a can include at least one selection transistor, one current control transistor (driving transistor), and a capacitor for one light-emitting device. A gate signal is input to a gate of the selection transistor, and a video signal is input to a source or a drain of the selection transistor. With such a structure, an active-matrix display apparatus is obtained.

The circuit portion 282 includes a circuit for driving the pixel circuits 283a in the pixel circuit portion 283. For example, the circuit portion 282 preferably includes one or both ofa gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high. For example, the aperture ratio of the display portion 281 can be higher than or equal to 40% and lower than 100%, preferably higher than or equal to 50% and lower than or equal to 95%, further preferably higher than or equal to 60% and lower than or equal to 95%, Furthermore, the pixels 284a can be arranged extremely densely and thus the display portion 281 can have significantly high resolution. For example, the pixels 284a are preferably arranged in the display portion 281 with a resolution higher than or equal to 2000 ppi, further preferably higher than or equal to 3000 ppi, still further preferably higher than or equal to 5000 ppi, yet still further preferably higher than or equal to 6000 ppi, and lower than or equal to 20000 ppi or lower than or equal to 30000 ppi.

Such a display module 280 has extremely high resolution, and thus can be suitably used for a VR device such as an HMD or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic appliances including a relatively small display portion. For example, the display module 280 can be favorably used in a display portion of a wearable electronic appliance, such as a wrist watch.

### [Display Apparatus 100A]

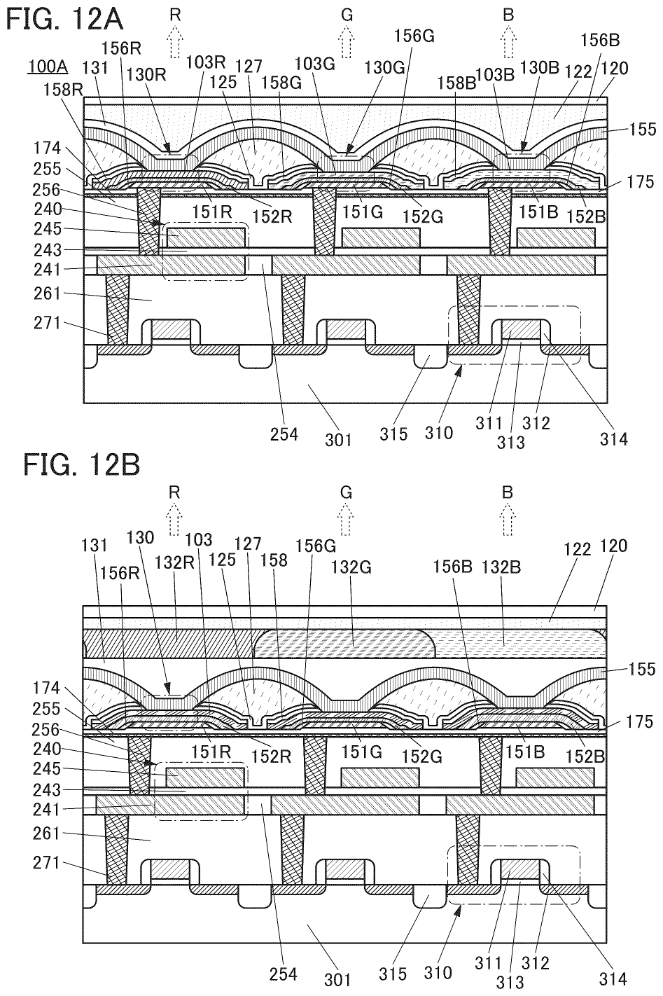

The display apparatus 100A illustrated in FIG. 12A includes a substrate 301, the light-emitting devices 130R, 130G, and 130B, a capacitor 240, and a transistor 310.

The substrate 301 corresponds to the substrate 291 in FIGS. 11A and 11B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is located between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 sandwiched therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. FIG. 12A illustrates an example in which the light-emitting devices 130R, 130G, and 130B each have the stacked-layer structure illustrated in FIG. 2B. An insulator is provided in regions between adjacent light-emitting devices. For example, in FIG. 12A, the inorganic insulating layer 125 and the insulating layer 127 over the inorganic insulating layer 125 are provided in those regions.

The insulating layer 156R is provided to include a region overlapping with the side surface of the conductive layer 1511R of the light-emitting device 130R. The insulating layer 156G is provided to include a region overlapping with the side surface of the conductive layer 151G of the light-emitting device 130G. The insulating layer 156B is provided to include a region overlapping with the side surface of the conductive layer 151B of the light-emitting device 130B. The conductive layer 152R is provided to cover the conductive layer 151R and the insulating layer 156R. The conductive layer 152G is provided to cover the conductive layer 151G and the insulating layer 156G. The conductive layer 15213 is provided to cover the conductive layer 151B and the insulating layer 156B. The sacrificial layer 158R is located over the organic compound layer 103R of the light-emitting device 130R. The sacrificial layer 158G is located over the organic compound layer 103G of the light-emitting device 130G. The sacrificial layer 158B is located over the organic compound layer 103B of the light-emitting device 130B.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. The top surface of the insulating layer 175 and the top surface of the plug 256 are level with or substantially level with each other. Any of a variety of conductive materials can be used for the plugs.

The protective layer 131 is provided over the light-emitting devices 130R, 130G, and 130B. A substrate 120 is attached to the protective layer 131 with the resin layer 122. Embodiment 3 can be referred to for the details of the light-emitting device 130 and the components thereover up to the substrate 120. The substrate 120 corresponds to the substrate 292 in FIG. 11A.

FIG. 12B illustrates a variation example of the display apparatus 100A illustrated in FIG. 12A. The display apparatus illustrated in FIG. 12B includes the coloring layers 132R, 132G, and 132B, and each of the light-emitting devices 130 includes a region overlapping with one of the coloring layers 132R, 132G, and 132B. In the display apparatus illustrated in FIG. 12B, the light-emitting device 130 can emit white light, for example. For example, the coloring layer 132R, the coloring layer 132G, and the coloring layer 132B can transmit red light, green light, and blue light, respectively.

This embodiment can be combined as appropriate with any of the other embodiments and examples. In this specification, in the case where a plurality of structure examples are shown in one embodiment, the structure examples can be combined as appropriate.

### Embodiment 6

In this embodiment, electronic appliances of embodiments of the present invention will be described.

Electronic appliances of this embodiment include the display apparatus of one embodiment of the present invention in their display portions. The display apparatus of one embodiment of the present invention is highly reliable and can be easily increased in resolution and definition. Thus, the display apparatus of one embodiment of the present invention can be used for display portions of a variety of electronic appliances.

Examples of the electronic appliances include a digital camera, a digital video camera, a digital photo frame, a mobile phone, a portable game console, a portable information terminal, and an audio reproducing device, in addition to electronic appliances with a relatively large screen, such as a television device, desktop and notebook personal computers, a monitor of a computer and the like, digital signage, and a large game machine such as a pachinko machine.

In particular, the display apparatus of one embodiment of the present invention can have high resolution, and thus can be favorably used for an electronic appliance having a relatively small display portion. Examples of such an electronic appliance include watch-type and bracelet-type information terminal devices (wearable devices) and wearable devices capable of being worn on a head, such as a VR device like a head-mounted display, a glasses-type AR device, and an MR device.

The definition of the display apparatus of one embodiment of the present invention is preferably as high as HD (number of pixels: 1280×720), FHD (number of pixels: 1920×1080), WQH D (number of pixels: 2560×1440), WQXGA (number of pixels: 2560×1600), 4K (number of pixels: 3840×2160), or 8K (number of pixels: 7680×4320). In particular, definition of 4K, 8K, or higher is preferable. The pixel density (resolution) of the display apparatus of one embodiment of the present invention is preferably higher than or equal to 100 ppi, further preferably higher than or equal to 300 ppi, further preferably higher than or equal to 500 ppi, further preferably higher than or equal to 1000 ppi, still further preferably higher than or equal to 2000 ppi, yet still further preferably higher than or equal to 3000 ppi, yet still further preferably higher than or equal to 5000 ppi, yet still further preferably higher than or equal to 7000 ppi. With such a display apparatus having one or both of high definition and high resolution, the electronic appliance can provide higher realistic sensation, sense of depth, and the like in personal use such as portable use or home use. There is no particular limitation on the screen ratio (aspect ratio) of the display apparatus of one embodiment of the present invention. For example, the display apparatus is compatible with a variety of screen ratios such as 1:1 (a square), 4:3, 16:9, and 16:10.

The electronic appliance in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

The electronic appliance in this embodiment can have a variety of functions. For example, the electronic appliance in this embodiment can have a function of displaying a variety of information (e.g., a still image, a moving image, and a text image) on the display portion, a touch panel function, a function of displaying a calendar, date, time, and the like, a function of executing a variety of software (programs), a wireless communication function, and a function of reading out a program or data stored in a recording medium.

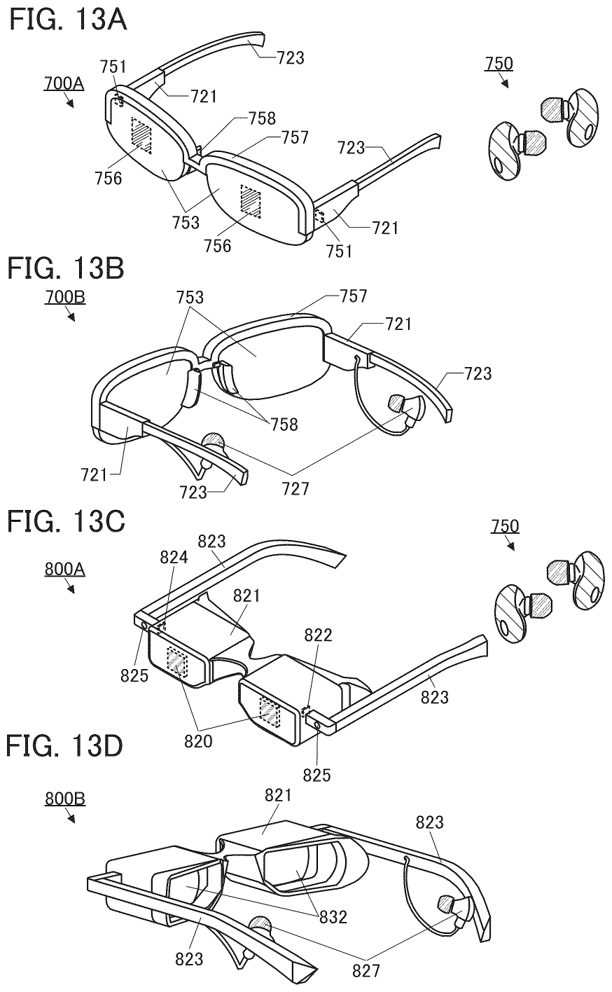

Examples of head-mounted wearable devices are described with reference to FIGS. 13A to 13D. These wearable devices have at least one of a function of displaying AR contents, a function of displaying VR contents, a function of displaying SR contents, and a function of displaying MR contents. The electronic appliance having a function of displaying contents of at least one of AR, VR, SR, MR, and the like enables the user to feel a higher level of immersion.

An electronic appliance 700A illustrated in FIG. 13A and an electronic appliance 700B illustrated in FIG. 13B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The display apparatus of one embodiment of the present invention can be used for the display panels 751. Thus, the electronic appliances can be highly reliable.

The electronic appliances 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753. Accordingly, the electronic appliances 700A and 700B are electronic appliances capable of performing AR display.

In the electronic appliances 700A and 700B, a camera capable of capturing images of the front side may be provided as the image capturing portion. Furthermore, when the electronic appliances 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic appliances 700A and 700B are provided with a battery, so that they can be charged wirelessly and/or by wire.

A touch sensor module may be provided in the housing 721. The touch sensor module has a function of detecting a touch on the outer surface of the housing 721. Detecting a tap operation, a slide operation, or the like by the user with the touch sensor module enables various types of processing. For example, a moving image can be paused or restarted by a tap operation, and can be fast-forwarded or fast-reversed by a slide operation. When the touch sensor module is provided in each of the two housings 721, the range of the operation can be increased.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

In the case of using an optical touch sensor, a photoelectric conversion device (also referred to as a photoelectric conversion element) can be used as a light-receiving element. One or both of an inorganic semiconductor and an organic semiconductor can be used for an active layer of the photoelectric conversion device.

An electronic appliance 800A illustrated in FIG. 13C and an electronic appliance 800B illustrated in FIG. 13D each include a pair of display portions 820, a housing 821, a communication portion 822, a pair of wearing portions 823, a control portion 824, a pair of image capturing portions 825, and a pair of lenses 832.

The display apparatus of one embodiment of the present invention can be used in the display portions 820. Thus, the electronic appliances can be highly reliable.

The display portions 820 are located inside the housing 821 so as to be seen through the lenses 832. When the pair of display portions 820 display different images, three-dimensional display using parallax can be performed.

The electronic appliances 800A and 800B can be regarded as electronic appliances for VR. The user who wears the electronic appliance 800A or 800B can see images displayed on the display portions 820 through the lenses 832.

The electronic appliances 800A and 800B preferably include a mechanism for adjusting the lateral positions of the lenses 832 and the display portions 820 so that the lenses 832 and the display portions 820 are located optimally in accordance with the positions of the user's eyes. Moreover, the electronic appliances 800A and 800B preferably include a mechanism for adjusting focus by changing the distance between the lenses 832 and the display portions 820.

The electronic appliance 800A or 800B can be mounted on the user's head with the wearing portions 823. FIG. 13C, for instance, shows an example where the wearing portion 823 has a shape like a temple (also referred to as a joint or the like) of glasses; however, one embodiment of the present invention is not limited thereto. The wearing portion 823 may have any shape with which the user can wear the electronic appliance, for example, a shape of a helmet or a band.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to cover a plurality of fields of view, such as a telescope field of view and a wide field of view.

Although an example where the image capturing portions 825 are provided is described here, a range sensor (hereinafter also referred to as a sensing portion) capable of measuring the distance between the user and an object just needs to be provided. In other words, the image capturing portion 825 is one embodiment of the sensing portion. As the sensing portion, an image sensor or a range image sensor such as a light detection and ranging (LiDAR) sensor can be used, for example. By using images obtained by the camera and images obtained by the range image sensor, more information can be obtained and a gesture operation with higher accuracy is possible.

The electronic appliance 800A may include a vibration mechanism that functions as bone-conduction earphones. For example, at least one of the display portion 820, the housing 821, and the wearing portion 823 can include the vibration mechanism. Thus, without additionally requiring an audio device such as headphones, earphones, or a speaker, the user can enjoy video and sound only by wearing the electronic appliance 800A.

The electronic appliances 800A and 800B may each include an input terminal. To the input terminal, a cable for supplying a video signal from a video output device or the like, power for charging a battery provided in the electronic appliance, and the like can be connected.

The electronic appliance of one embodiment of the present invention may have a function of performing wireless communication with earphones 750. The earphones 750 include a communication portion (not illustrated) and have a wireless communication function. The earphones 750 can receive information (e.g., audio data) from the electronic appliance with the wireless communication function. For example, the electronic appliance 700A in FIG. 13A has a function of transmitting information to the earphones 750 with the wireless communication function. For another example, the electronic appliance 800A in FIG. 13C has a function of transmitting information to the earphones 750 with the wireless communication function.

The electronic appliance may include an earphone portion. The electronic appliance 700B in FIG. 13B includes earphone portions 727. For example, the earphone portion 727 can be connected to the control portion by wire. Part of a wiring that connects the earphone portion 727 and the control portion may be located inside the housing 721 or the wearing portion 723.

Similarly, the electronic appliance 800B in FIG. 13D includes earphone portions 827. For example, the earphone portion 827 can be connected to the control portion 824 by wire. Part of a wiring that connects the earphone portion 827 and the control portion 824 may be located inside the housing 821 or the wearing portion 823. Alternatively, the earphone portions 827 and the wearing portions 823 may include magnets. This structure is preferably employed, in which case the earphone portions 827 can be fixed to the wearing portions 823 with magnetic force and thus can be easily housed.

The electronic appliance may include an audio output terminal to which earphones, headphones, or the like can be connected. The electronic appliance may include one or both of an audio input terminal and an audio input mechanism. As the audio input mechanism, a sound collecting device such as a microphone can be used, for example. The electronic appliance may have a function of a headset by including the audio input mechanism.

As described above, both the glasses-type device (e.g., the electronic appliances 700A and 700B) and the goggles-type device (e.g., the electronic appliances 800A and 800B) are preferable as the electronic appliance of one embodiment of the present invention.

The electronic appliance of one embodiment of the present invention can transmit information to earphones by wire or wirelessly.

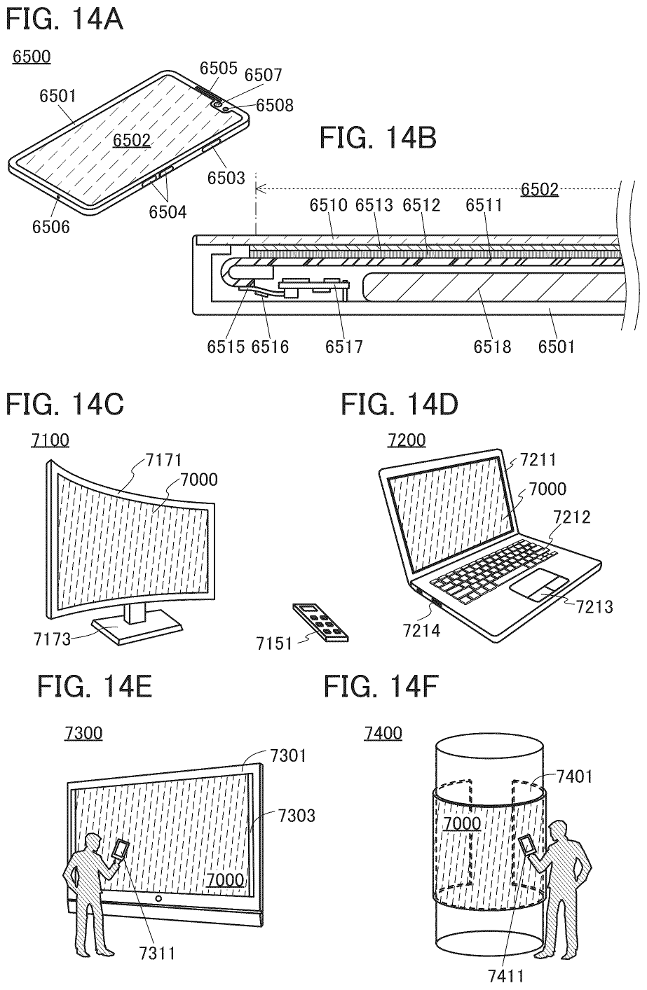

An electronic appliance 6500 illustrated in FIG. 14A is a portable information terminal that can be used as a smartphone.

The electronic appliance 6500 includes a housing 6501, a display portion 6502, a power button 6503, buttons 6504, a speaker 6505, a microphone 6506, a camera 6507, a light source 6508, and the like. The display portion 6502 has a touch panel function.

The display apparatus of one embodiment of the present invention can be used in the display portion 6502. Thus, the electronic appliance can be highly reliable.

FIG. 14B is a schematic cross-sectional view including an end portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with an adhesive layer (not illustrated).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

The display apparatus of one embodiment of the present invention can be used in the display panel 6511. Thus, the electronic appliance can be extremely lightweight. Since the display panel 6511 is extremely thin, the battery 6518 with high capacity can be mounted without an increase in the thickness of the electronic appliance. The electronic appliance can have a narrow bezel when part of the display panel 6511 is folded back so that the portion connected to the FPC 6515 is provided on the back side of a pixel portion.

FIG. 14C illustrates an example of a television device. In a television device 7100, a display portion 7000 is incorporated in a housing 7171. Here, the housing 7171 is supported by a stand 7173.

The display apparatus of one embodiment of the present invention can be used in the display portion 7000. Thus, a highly reliable electronic appliance can be obtained.

Operation of the television device 7100 illustrated in FIG. 14C can be performed with an operation switch provided in the housing 7171 and a separate remote control 7151. Alternatively, the display portion 7000 may include a touch sensor, and the television device 7100 may be operated by touch on the display portion 7000 with a finger or the like. The remote control 7151 may be provided with a display portion for displaying information output from the remote control 7151. With operation keys or a touch panel of the remote control 7151, channels and volume can be controlled and video displayed on the display portion 7000 can be controlled.

Note that the television device 7100 includes a receiver, a modem, and the like. A general television broadcast can be received with the receiver. When the television device is connected to a communication network with or without wires via the modem, one-way (from a transmitter to a receiver) or two-way (e.g., between a transmitter and a receiver or between receivers) information communication can be performed.

FIG. 14D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The display apparatus of one embodiment of the present invention can be used in the display portion 7000. Thus, a highly reliable electronic appliance can be obtained.

FIGS. 14E and 14F illustrate examples of digital signage.

Digital signage 7300 illustrated in FIG. 14E includes a housing 7301, the display portion 7000, a speaker 7303, and the like. The digital signage 7300 can also include an LED lamp, operation keys (including a power switch or an operation switch), a connection terminal, a variety of sensors, a microphone, and the like.

FIG. 14F shows digital signage 7400 attached to a cylindrical pillar 7401. The digital signage 7400 includes the display portion 7000 provided along a curved surface of the pillar 7401.

In FIGS. 14E and 14F, the display apparatus of one embodiment of the present invention can be used in the display portion 7000. Thus, a highly reliable electronic appliance can be obtained.

A larger area of the display portion 7000 can increase the amount of information that can be provided at a time. The display portion 7000 having a larger area attracts more attention, so that the effectiveness of the advertisement can be increased, for example.

The touch panel is preferably used in the display portion 7000, in which case in addition to display of still or moving images on the display portion 7000, intuitive operation by a user is possible. Moreover, in the case of an application for providing information such as route information or traffic information, usability can be enhanced by intuitive operation.

As illustrated in FIGS. 14E and 14F, it is preferable that the digital signage 7300 or the digital signage 7400 can work with an information terminal 7311 or an information terminal 7411, such as a smartphone that a user has, through wireless communication. For example, information of an advertisement displayed on the display portion 7000 can be displayed on a screen of the information terminal 7311 or the information terminal 7411. By operation of the information terminal 7311 or the information terminal 7411, a displayed image on the display portion 7000 can be switched.

It is possible to make the digital signage 7300 or the digital signage 7400 execute a game with the use of the screen of the information terminal 7311 or the information terminal 7411 as an operation means (controller). Thus, an unspecified number of users can join in and enjoy the game concurrently.

This embodiment can be combined as appropriate with any of the other embodiments and examples. In this specification, in the case where a plurality of structure examples are shown in one embodiment, the structure examples can be combined as appropriate.

### Example 1

### Synthesis Example 1

In this example, a method for synthesizing 4,7-bis[4-(1-pyrrolidinyl)phenyl]-1,10-phenanthroline (abbreviation: PrdP2Phen) represented by Structural Formula (100) in Embodiment 1 is described. The structural formula of PrdP2Phen is shown below.

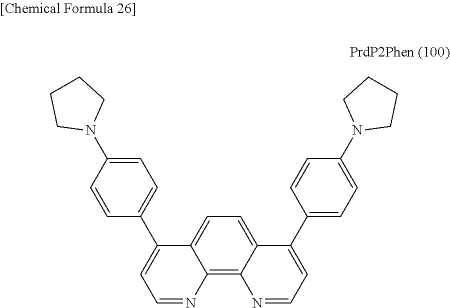 [SMILES: c1cc(-c2ccc(N3CCCC3)cc2)c2ccc3c(-c4ccc(N5CCCC5)cc4)ccnc3c2n1]

### <Synthesis of PrdP2Phen>

In a 100-mL three-neck flask were put 1.4 g (4.2 mmol) of 4,7-dibromo-1,10-phenanthroline, 2.5 g (9.2 mmol) of 2-[4-(1-pyrrolidinyl)phenyl]-4,4,5,5-tetramethyl-1,3,2-dioxaborolane, 0.48 mL (0.29 mmol) of tricyclohexylphosphine (abbreviation: Cy₃P) accounting for approximately 18% in toluene solution, 3.0 g (14 mmol) of tripotassium phosphate, 25 mL of 1,4-dioxane, and 12 mL of water, and the mixture was degassed by being stirred under reduced pressure. To this mixture, 0.12 g (0.13 mmol) of tris(dibenzylideneacetone)dipalladium(0) (abbreviation: Pd₂(dba)₃) was added and stirring was performed at 100° C. under a nitrogen stream for 12 hours. After the stirring, the mixture was cooled down to room temperature. A precipitated solid of this mixture was collected by suction filtration. To this solid, 1,4-dioxane was added, irradiation with ultrasonic waves was performed, and the solid was collected by suction filtration. Chloroform was added to this solid to dissolve the solid. To this solution, water was added and an organic layer was subjected to extraction with chloroform. The extracted solution was concentrated to give a solid. To this solid, toluene was added, irradiation with ultrasonic waves was performed, and the solid was collected by suction filtration, whereby 1.2 g of a target pale yellow solid was obtained in a yield of 60%. A synthesis scheme of PrdP2Phen is shown in Formula (a-1) below.

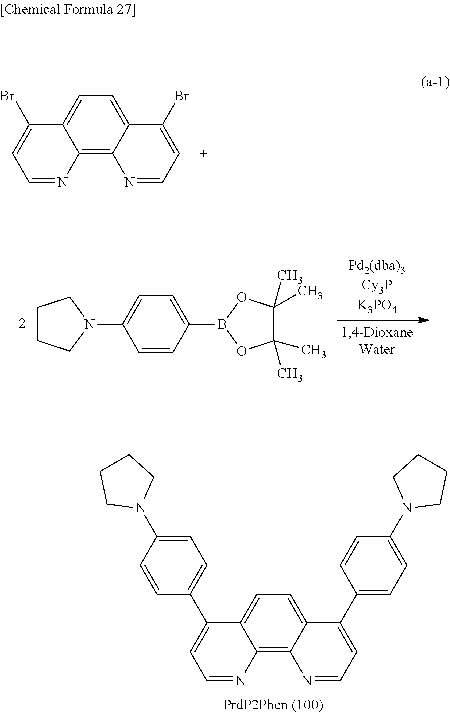

By a train sublimation method, 1.2 g of the obtained pale yellorw solid was purified by sublimation. In the purification by sublimation, heating was performed for 20 hours at an argon flow rate of 18 mL/min, a pressure of 3.7 Pa, and a heating temperature of 280° C. As a result, 0.71 g of a target yellow solid was obtained at a collection rate of 59%.

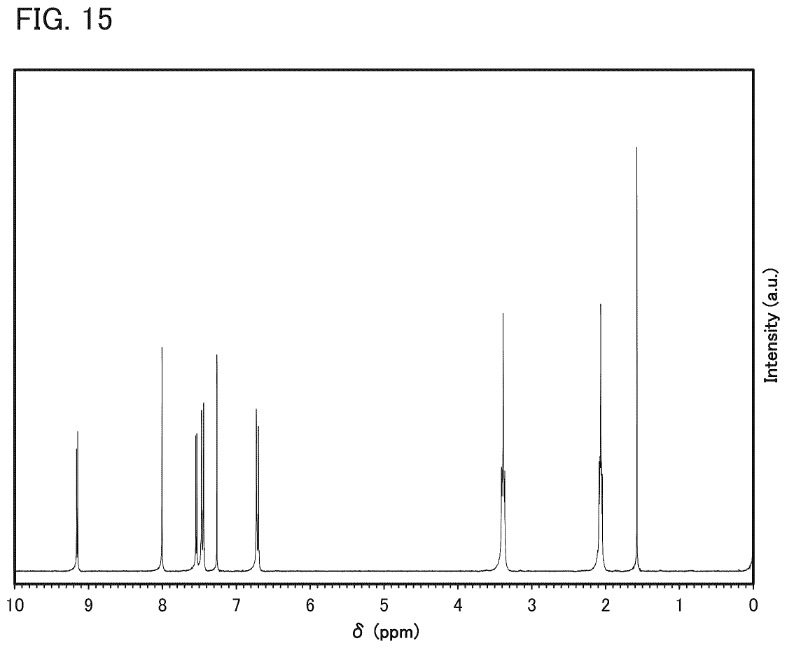

FIG. 15 shows a ¹H NMR spectrum of PrdP2Phen after the purification by sublimation. Results of ¹H NMR measurement are shown below. The results show that PrdP2Phen was obtained.

¹H NMR (CDCU1, 300 MHz): δ 9.15 (d, J=4.5 Hz, 2H), 8.00 (s, 2H), 7.54 (d, J=4.5 Hz, 2H), 7.46 (d, J=8.7 Hz, 4H), 6.71 (d, J=8.4 Hz, 4H), 3.41-3.37 (m, 8H), 2.09-2.04 (m, 8H).

### <T_{g} Measurement of PrdP2Phen>

T_{g} of PrdP2Phen was measured. Note that T_{g} was measured with a differential scanning calorimeter (DSC8500, manufactured by PerkinElmer Japan Co., Ltd.) in a state where a powder was put on an aluminum cell and the temperature was increased at a rate of 40° C./min. As a result, T_{g} of PrdP2Phen was 130° C.

Next, a solubility test of PrdP2Phen was performed. Note that the solubility test was conducted at a pressure of one atmosphere, at room temperature (RT).

### <Solubility Test of PrdP2Phen by LC-MS Analysis>

In the LC-MS analysis, liquid chromatography (LC) separation was carried out with ACQUITY UPLC manufactured by Waters Corporation, and MS analysis (mass spectrometry) was carried out with Xevo G2 Tof MS manufactured by Waters Corporation. Acquity UPLC BEH C8 (2.1×100 mm, 1.7 μm) was used as a column for the LC separation. Acetonitrile was used for Mobile Phase A and a 0.1% aqueous solution of formic acid was used for Mobile Phase B. The injection amount of the sample was 5.0 μL. Note that in the analysis, the wavelength of a photodiode array detector was set to 263 nm±1 nm.

In a 5-mL sample bottle, I mg of PrdP2Phen was put and 1 mL of water was added thereto. This mixture was irradiated with ultrasonic waves for five minutes. This mixture was filtered through a membrane filter to remove the solid, and the resulting filtrate was diluted by five times with acetonitrile. The obtained solution was subjected to LC-MS analysis.

As a result, the peak area value derived from PrdP2Phen failed to be obtained through the LC-MS analysis.

It is thus found that the organic compound of one embodiment of the present invention can be favorably used for a light-emitting device whose fabrication process includes processing using water or a chemical solution containing water as a solvent (i.e., a light-emitting device involving processing by a lithography method).

### Example 2

In this example, a light-emitting device I using 4,7-bis[4-(1-pyrrolidinyl)phenyl]-1,10-phenanthroline (abbreviation: PrdP2Phen) (Structural Formula (100)), which is the organic compound of one embodiment of the present invention, and a comparative light-emitting device 2 using a comparative organic compound were fabricated and the comparison results of the device characteristics are described. The light-emitting device 1 and the comparative light-emitting device 2 were fabricated by a method that includes a step of exposing an organic compound layer to the air, which is modeled on a method that includes a process in which an organic compound layer is formed through processing by a photolithography method. The structural formulae of the organic compounds used in the light-emitting device 1 and the comparative light-emitting device 2 are shown below.

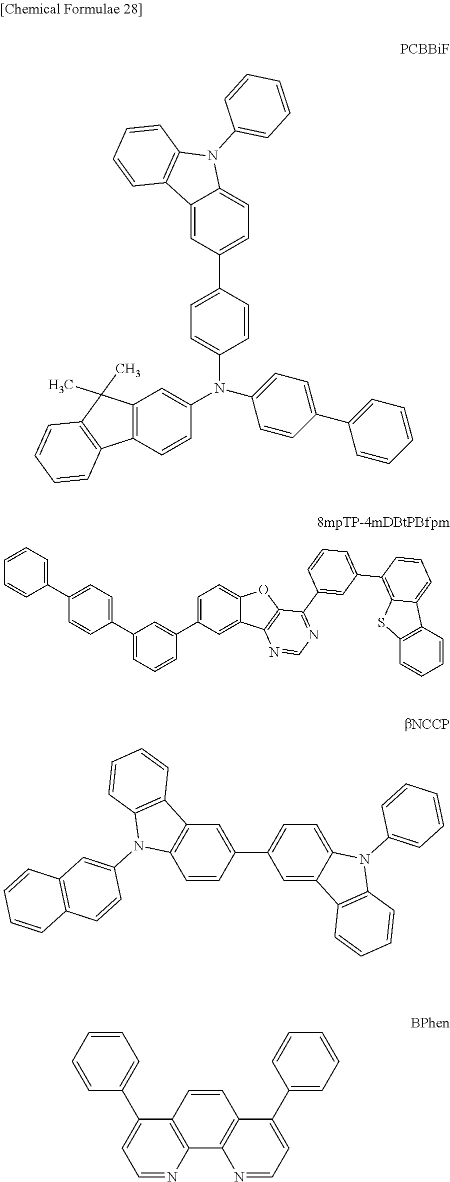 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(-c3cccc(-c4ccc5oc6c(-c7cccc(-c8cccc9c8sc8ccccc89)c7)ncnc6c5c4)c3)cc2)cc1 ; c1ccc(-n2c3ccccc3c3cc(-c4ccc5c(c4)c4ccccc4n5-c4ccc5ccccc5c4)ccc32)cc1 ; c1ccc(-c2ccnc3c2ccc2c(-c4ccccc4)ccnc23)cc1]

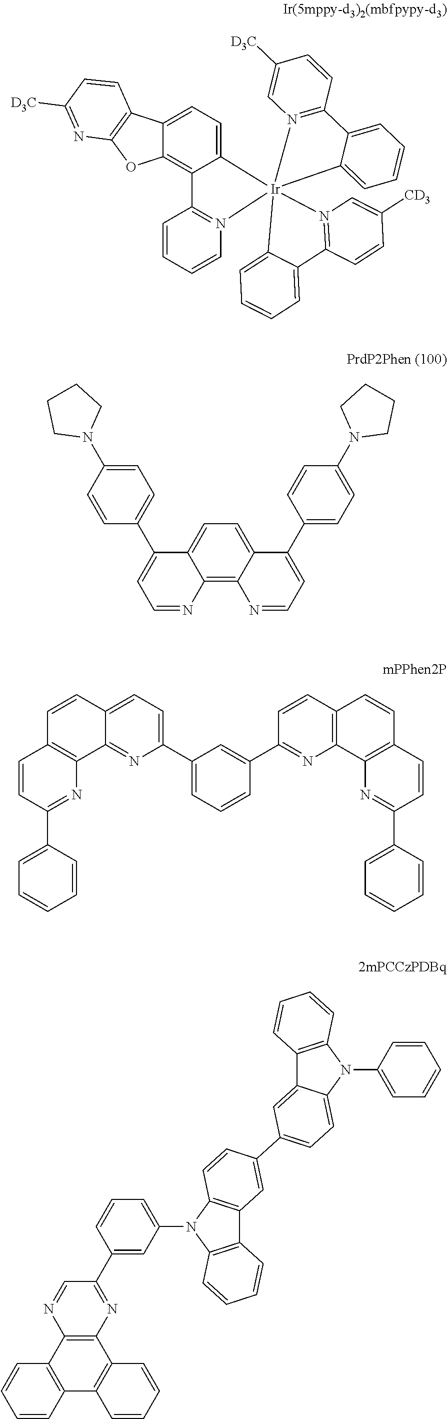

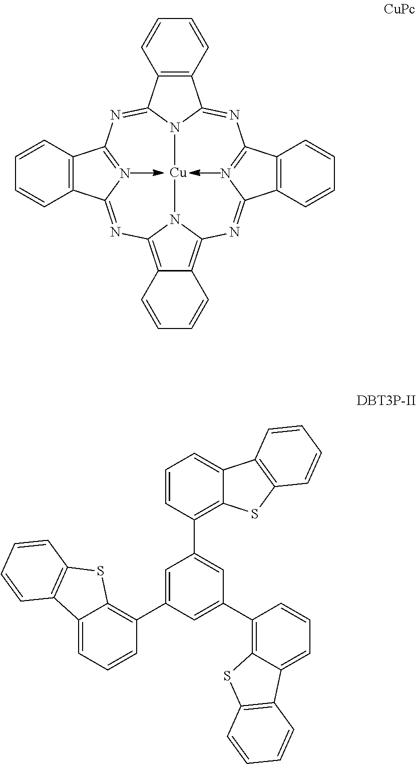

### (Method for Fabricating Light-Emitting Device 1)

First, as a reflective electrode, silver (Ag) was deposited over a glass substrate to a thickness of 100 nm by a sputtering method, and then, as a transparent electrode, indium tin oxide containing silicon oxide (ITSO) was deposited to a thickness of 85 nm by a sputtering method, so that a first electrode was formed. The electrode area was set to 4 mm² (2 mm×2 mm). Note that the transparent electrode functions as an anode, and the transparent electrode and the reflective electrode can be collectively regarded as the first electrode.

Next, in pretreatment for forming the light-emitting device over the substrate, the surface of the substrate was washed with water, and baking was performed at 200° C. for I hour.

After that, the substrate was transferred into a vacuum evaporation apparatus where the pressure had been reduced to approximately 1×10⁻⁴ Pa, and was subjected to heat treatment at 170° C. for 30 minutes in a heating chamber of the vacuum evaporation apparatus, and then the substrate was cooled down for approximately 30 minutes.

Then, the substrate was fixed to a substrate holder provided in the vacuum evaporation apparatus such that the surface on which the first electrode was formed faced downward. Over the first electrode, N-(biphenyl-4-yl)-N-[4-(9-phenyl-9H-carbazol-3-yl)phenyl]-9,9-dimethyl-9H-fluoren-2-amine (abbreviation: PCBBiF) and a fluorine-containing electron acceptor material with a molecular weight of 672 and (OCHD-003) were deposited by co-evaporation to a thickness of 10 nm such that the weight ratio of PCBBiF to OCHD-003 was 1:0.03, so that a hole-injection layer was formed.

Over the hole-injection layer, PCBBiF was deposited by evaporation to a thickness of 90 nm, so that a first hole-transport layer was formed.

Then, over the first hole-transport layer, 8-(1,1′: 4′, 1″-terphenyl-3-yl)-4-[3-(dibenzothiophen-4-yl)phenyl]-[1]benzofuro[3,2-d]pyrimidine (abbreviation: 8mpTP-4mDBtPBfpm), 9-(2-naphthyl)-9′-phenyl-9H,9′H-3,3′-bicarbazole (abbreviation: βNCCP), and [2-d₃-methyl-8-(2-pyridinyl-KN)benzofuro[2,3-b]pyridine-KC]bis[2-(5-d₃-methyl-2-pyridinyl-κN²)phenyl-κC]iridium(III) (abbreviation: Ir(5mppy-d₃)₂(mbfpypy-d₃)) were deposited by co-evaporation to a thickness of 40 nm such that the weight ratio of 8mpTP-4mDBtPBfpm to βNCCP to Ir(5mppy-d₃)₂(mbfpypy-d₃) was 0.5:0.5:0.1, whereby a first light-emitting layer was formed.

After that, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 10 nm, so that a first electron-transport layer was formed.

After the formation of the first electron-transport layer, PrdP2Phen, which is the organic compound of one embodiment of the present invention, and indium (In) were deposited by co-evaporation to a thickness of 5 nm such that the volume ratio of PrdP2Phen to In was 1.0:0.1, whereby a first layer of an intermediate layer was formed.

Then, a film of copper phthalocyanine (abbreviation: CuPc) was formed to have a thickness of 2 nm, so that a third layer of the intermediate layer was formed.

Furthermore, PCBBiF and OCHI-D-003 were deposited by co-evaporation to a thickness of 10 nm such that the weight ratio of PCBBiF to OCHD-003 was 1:0.15, whereby a second layer of the intermediate layer was formed.

Next, over the intermediate layer, PCBBiF was deposited by evaporation to a thickness of 55 nm, so that a second hole-transport layer was formed.

Over the second hole-transport layer, 8mpTP-4mDBtPBfpm, βNCCP, and Ir(5mppy-d₃)₂(mbfpypy-d₃) were deposited by co-evaporation to a thickness of 40 nm such that the weight ratio of 8mpTP-4mDBtPBfpm to βNCCP to Ir(5mppy-d₃)₂(mbfpypy-d₃) was 0.5:0.5:0.1, whereby a second light-emitting layer was formed.

After that, 2mPCCzPDBq was deposited by evaporation to a thickness of 20 nm and 2,2′-(1,3-phenylene)bis(9-phenyl-1,10-phenanthroline) (abbreviation: mPPhen2P) was further deposited by evaporation to a thickness of 20 nm, so that a second electron-transport layer was formed.

By a method that is modeled on the method for forming an organic compound layer through processing by a photolithography method, the organic compound layer was exposed to the air after the formation of the second electron-transport layer. Then, the substrate was transferred into a vacuum evaporation apparatus where the pressure had been reduced to approximately 1×10⁻⁴ Pa, and heat treatment was performed at 110° C. for 1 hour in a heating chamber of the vacuum evaporation apparatus.

After that, over the second electron-transport layer, lithium fluoride (LiF) and ytterbium (Yb) were deposited by co-evaporation to a thickness of 1.5 nm such that the volume ratio of LiF to Yb was 1:0.5 to form an electron-injection layer, and lastly silver (Ag) and magnesium (Mg) were deposited by co-evaporation to a thickness of 15 nm such that the volume ratio of Ag to Mg was 1:0.1 to form a second electrode, whereby the light-emitting device 1 was fabricated.

The second electrode is a semi-transmissive and semi-reflective electrode having a function of reflecting light and a function of transmitting light; thus, the light-emitting device of this example is a top-emission tandem device in which light is extracted through the second electrode. Over the second electrode, 4,4′, 4″-(benzene-1,3,5-triyl)tri(dibenzothiophene) (abbreviation: DBT3P-II) was deposited by evaporation to a thickness of 70 nm as a cap layer to improve light extraction efficiency.

### (Method for Fabricating Comparative Light-Emitting Device 2)

The comparative light-emitting device 2 is different from the light-emitting device 1 in that not PrdP2Phen but 4,7-diphenyl-1,10-phenanthroline (abbreviation: BPhen) was used in the first layer of the intermediate layer. The other components were formed in the same manner as those in the light-emitting device 1.

The table below lists the structures of the light-emitting device 1 and the comparative light-emitting device 2.

TABLE 1

| | | | | Light-emitting | Comparative light- |
|---|---|---|---|---|---|
| | | Thickness | | device 1 | emitting device 2 |
| Cap layer | | 70 | nm | DBT3P-II | |
| Second electrode | | 15 | nm | Ag:Mg (1:0.1) | |
| Electron-injection layer | | 1.5 | nm | LiF:Yb (1:0.5) | |
| — | | | | Air exposure | |
| Second electron- | 2 | 20 | nm | mPPhen2P | |
| transport layer | 1 | 20 | nm | 2mPCCzPDBq | |
| Second light-emitting layer | | 40 | nm | 8mpTP-4mDBtPBfpm:βNCCP:Ir(5mppy-d₃)₂(mbfpypy-d₃) | |
| | | | | (0.5:0.5:0.1) | |
| Second hole-transport layer | | 55 | nm | PCBBiF | |
| Intermediate layer | Second layer | 10 | nm | PCBBiF:OCHD-003 (1:0.15) | |
| | Third layer | 2 | nm | CuPc | |
| | First layer | 5 | nm | PrdP2Phen:In (1.0:0.1) | Bphen:In (1.0:0.1) |

| First electron-transport layer | | 10 | nm | 2mPCCzPDBq |
|---|---|---|---|---|
| First light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| First hole-transport layer | | 90 | nm | PCBBiF |
| Hole-injection layer | | 10 | nm | PCBBiF:OCHD-003 (1:0.03) |
| First electrode | 2 | 85 | nm | ITSO |
| | 1 | 100 | nm | Ag |

The light-emitting devices fabricated were each sealed using a glass substrate in a glove box containing a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for 1 hour. Then, the initial characteristics of the light-emitting devices were measured.

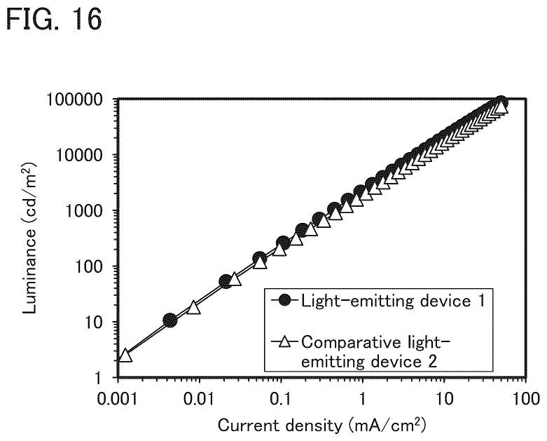

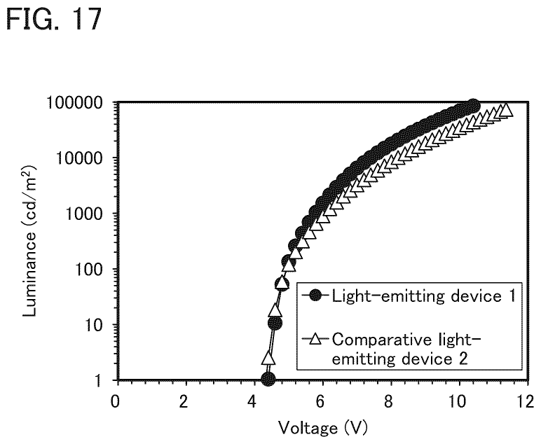

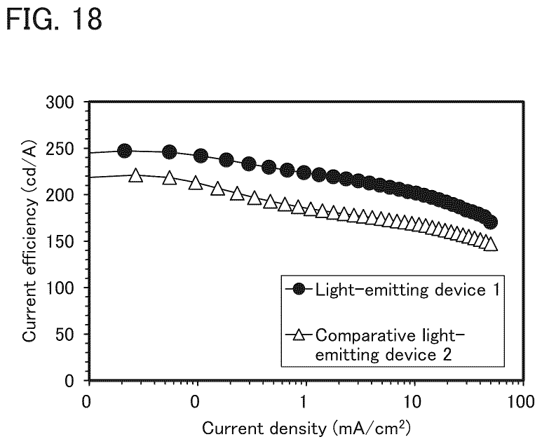

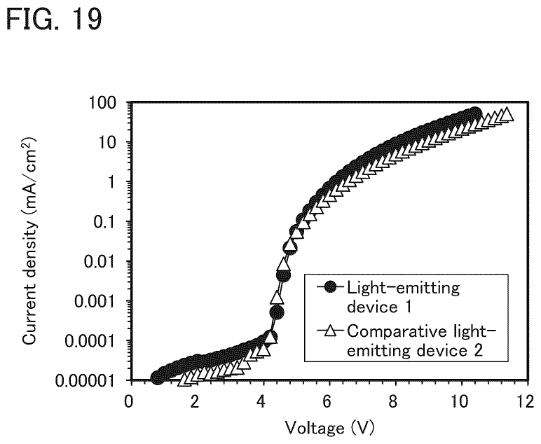

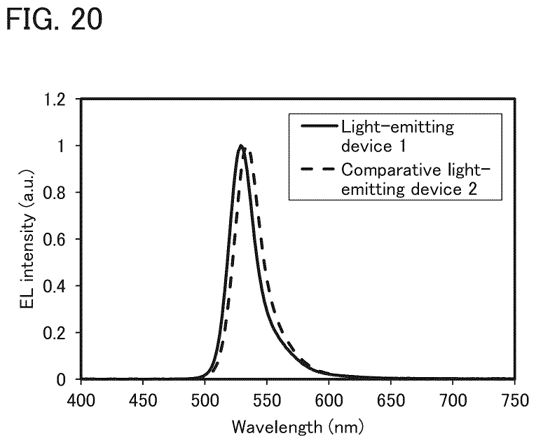

FIG. 16 shows the luminance-current density characteristics of the light-emitting device 1 and the comparative light-emitting device 2, FIG. 17 shows the luminance-voltage characteristics thereof, FIG. 18 shows the current efficiency-current density characteristics thereof, FIG. 19 shows the current density-voltage characteristics thereof, and FIG. 20 shows the electroluminescence spectra thereof.

The table below shows the main characteristics of the light-emitting device I and the comparative light-emitting device 2 at a luminance of approximately 1000 cd/m². The luminance, CIE chromaticity, and electroluminescence spectra were measured at normal temperature with a spectroradiometer (SR-UL1R manufactured by TOPCON TECHNOHOUSE CORPORATION).

TABLE 2

| | | | Current | | | | Current |
|---|---|---|---|---|---|---|---|
| | Voltage | Current | density | | | Luminance | efficiency |
| | (V) | (mA) | (mA/cm²) | Chromaticity x | Chromaticity y | (cd/m²) | (cd/A) |
| Light-emitting device 1 | 5.8 | 0.018 | 0.45 | 0.23 | 0.73 | 1040 | 229 |
| Comparative light- | 6.0 | 0.019 | 0.47 | 0.26 | 0.71 | 900 | 193 |
| emitting device 2 | | | | | | | |

FIGS. 16 to 20 and the above table reveal that the light-emitting device 1 and the comparative light-emitting device 2 exhibit green light emission derived from Ir(5mppy-d₃)₂(mbfpypy-d₃) and have tandem structures. The light-emitting device I has a lower driving voltage (see FIG. 17) and higher current efficiency, which is one of the indicators of emission efficiency, at the same current density (see FIG. 18) than the comparative light-emitting device 2; this shows that the light-emitting device 1 has favorable emission characteristics.

It is probable that the electron density of the phenanthroline ring is increased, so that the efficiency of the interaction between the phenanthroline ring and the metal is increased because PrdP2Phen used in the light-emitting device I has a pyrrolidine ring.

It was thus found that even when fabricated through a step of exposing the organic compound layer to the air, a light-emitting device having the structure of one embodiment of the present invention can have favorable characteristics. It was also found that a tandem light-emitting device with a low driving voltage and high emission efficiency can be fabricated using the organic compound of one embodiment of the present invention.

### Example 3

In this example, a light-emitting device 3 and a light-emitting device 4 each using PrdP2Phen (Structural Formula (100)), which is the organic compound of one embodiment of the present invention, were fabricated and the measurement results of the device characteristics are described. The light-emitting devices 3 and 4 were fabricated by a process in which an organic compound layer is formed through processing by a photolithography method. The structural formulae of the organic compounds used in the light-emitting devices 3 and 4 are shown below.

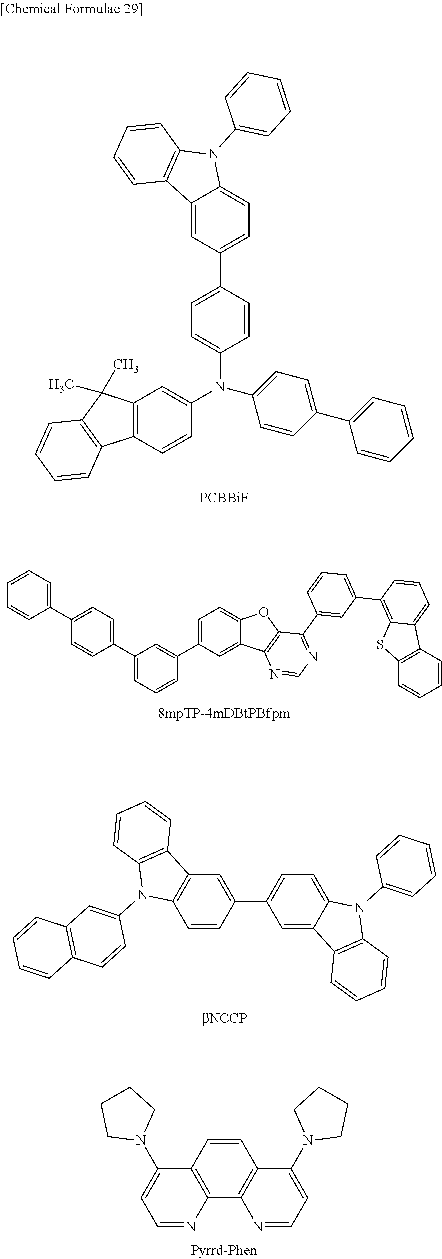

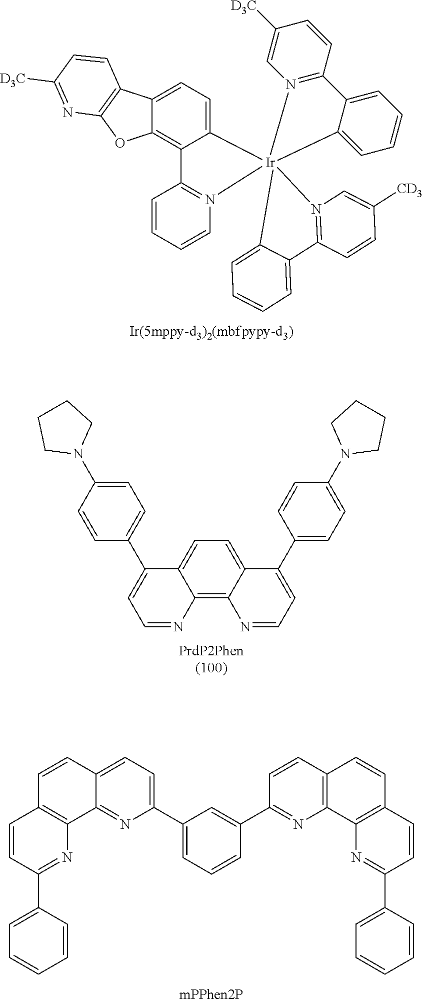

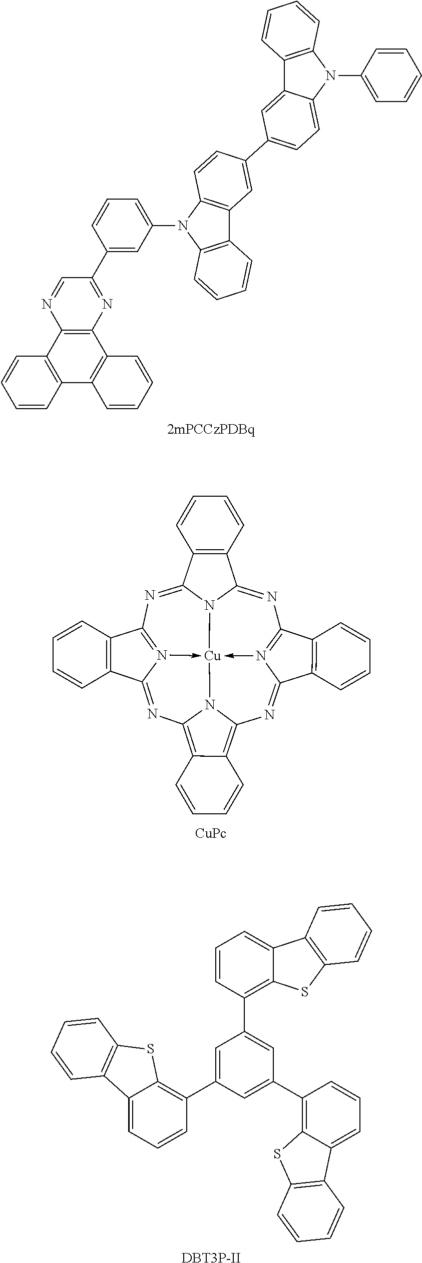

### (Method for Fabricating Light-Emitting Device 3)

First, as a reflective electrode, an alloy containing silver (Ag), palladium (Pd), and copper (Cu) (abbreviation: APC) was deposited over a glass substrate to a thickness of 100 nm by a sputtering method, and then, as a transparent electrode, indium tin oxide containing silicon oxide (ITSO) was deposited to a thickness of 50 nm by a sputtering method, so that the first electrode was formed. The electrode area was set to 4 mm² (2 mm×2 mm). Note that the transparent electrode functions as the anode, and the transparent electrode and the reflective electrode can be collectively regarded as the first electrode.

Next, in pretreatment for forming the light-emitting device over the substrate, the surface of the substrate was washed with water, and baking was performed at 200° C. for 1 hour.

After that, the substrate was transferred into a vacuum evaporation apparatus where the pressure had been reduced to approximately 1×10⁻⁴ Pa, and was subjected to heat treatment at 170° C. for 30 minutes in a heating chamber of the vacuum evaporation apparatus, and then the substrate was cooled down for approximately 30 minutes.

Then, the hole-injection layer was formed. The structure of the hole-injection layer is similar to that of the light-emitting device 1.

Over the hole-injection layer, PCBBiF was deposited by evaporation to a thickness of 120 nm, so that the first hole-transport layer was formed.

Next, the first light-emitting layer was formed over the first hole-transport layer, and then the first electron-transport layer was formed over the first light-emitting layer. The structures of the first light-emitting layer and the first electron-transport layer are similar to those of the light-emitting device 1.

After the formation of the first electron-transport layer, PrdP2Phen, which is the organic compound of one embodiment of the present invention, and indium (In) were deposited by co-evaporation to a thickness of 5 nm such that the volume ratio of PrdP2Phen to In was 1.0:0.05, whereby the first layer of the intermediate layer was formed.

Next, the third layer of the intermediate layer was formed, and then the second layer of the intermediate layer was formed. The structures of the third and second layers of the intermediate layer are similar to those of the light-emitting device 1.

Next, over the intermediate layer, PCBBiF was deposited by evaporation to a thickness of 50 nm, so that the second hole-transport layer was formed.

The second light-emitting layer was formed over the second hole-transport layer, and then the second electron-transport layer was formed. The structures of the second light-emitting layer and the second electron-transport layer are similar to those of the light-emitting device 1.

After the formation of the second electron-transport layer, processing by a photolithography method and heat treatment were performed.

### <<Processing by Photolithography Method and Heat Treatment>>

Here, the processing by a photolithography method and the heat treatment are described. First, the substrate was taken out from the vacuum evaporation apparatus and exposed to the air, and then aluminum oxide was deposited to a thickness of 30 nm by an ALD method using trimethylaluminum (abbreviation: TMA) as a precursor and water vapor as an oxidizer to form a first sacrificial layer.

Next, over the first sacrificial layer, molybdenum was deposited to a thickness of 50 nm by a sputtering method to form a second sacrificial layer.

A resist was formed using a photoresist over the second sacrificial layer, and processing was performed by a photolithography method to form a slit having a width of 3 μm in a position 3.5 μm away from an end portion of the first electrode.

Specifically, the second sacrificial layer was processed by using an etching gas containing carbon tetrafluoride (CF₄), oxygen (O₂), and helium (He) at a flow rate ratio of CF₄:O₂:He=100:67:333, then by using an etching gas containing oxygen with the use of the resists as masks. Next, the first sacrificial layer was processed using an etching gas containing fluoroform (CHF₃) and helium (He) at a flow rate ratio of CHF₃:He=1:49 with the use of the second sacrificial layer as a hard mask. After that, the organic compound layer was processed using an etching gas containing oxygen (O₂).

After the processing by a photolithography method, the second sacrificial layer was removed using an etching gas containing carbon tetrafluoride (CF₄), oxygen (O₂), and helium (He) at a flow rate ratio of CF₄:O₂:He=100:67:333. Then, the first sacrificial layer was removed using an acidic chemical solution containing water as a solvent and hydrofluoric acid, so that the top surface of the organic compound layer was exposed. Then, the substrate was transferred into a vacuum evaporation apparatus where the pressure had been reduced to approximately 1×10⁻⁴ Pa, and heat treatment was performed at 100° C. for 1 hour in a heating chamber of the vacuum evaporation apparatus.

The above is the description of the processing by a photolithography method and the heat treatment. As described above, the processing by a photolithography method, the heat treatment and the treatment using water or a chemical solution containing water as a solvent are performed.

After the processing by a photolithography method and the heat treatment, the electron-injection layer was formed over the top surface of the exposed organic compound layer, i.e., over the second electron-transport layer, and lastly the second electrode was formed, whereby the light-emitting device 3 was fabricated. The cap layer was formed over the second electrode. The structures of the electron-injection layer, the second electrode, and the cap layer are similar to those of the light-emitting device 1.

### (Method for Fabricating Light-Emitting Device 4)

The light-emitting device 4 is different from the light-emitting device 3 in that not In but ytterbium (Yb) was used in the first layer of the intermediate layer. The other components were formed in the same manner as those in the light-emitting device 3.

The table below lists the structures of the light-emitting devices 3 and 4.

TABLE 3

| | | Thickness | | Light-emitting device 3 | Light-emitting device 4 |
|---|---|---|---|---|---|
| Cap layer | | 70 | nm | DBT3P-II | |
| Second electrode | | 15 | nm | Ag:Mg (1:0.1) | |
| Electron-injection layer | | 1.5 | nm | LiF:Yb (1:0.5) | |
| — | | | | Processed by photolithography method | |
| Second electron- | 2 | 20 | nm | mPPhen2P | |
| transport layer | 1 | 20 | nm | 2mPCCzPDBq | |
| Second light-emitting layer | | 40 | nm | 8mpTP-4mDBtPBfpm:βNCCP:Ir(5mppy-d₃)₂(mbfpypy-d₃) | |
| | | | | (0.5:0.5:0.1) | |
| Second hole-transport layer | | 50 | nm | PCBBiF | |
| Intermediate layer | Second layer | 10 | nm | PCBBiF:OCHD-003 (1:0.15) | |
| | Third layer | 2 | nm | CuPc | |
| | First layer | 5 | nm | PrdP2Phen:In (1:0.05) | PrdP2Phen:Yb (1:0.02) |

| First electron-transport layer | | 10 | nm | 2mPCCzPDBq |
|---|---|---|---|---|
| First light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| First hole-transport layer | | 120 | nm | PCBBiF |
| Hole-injection layer | | 10 | nm | PCBBiF:OCHD-003 (1:0.03) |
| First electrode | 2 | 50 | nm | ITSO |
| | 1 | 100 | nm | APC |

The light-emitting devices fabricated were each sealed using a glass substrate in a glove box containing a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for 1 hour. Then, the initial characteristics of the light-emitting devices were measured.

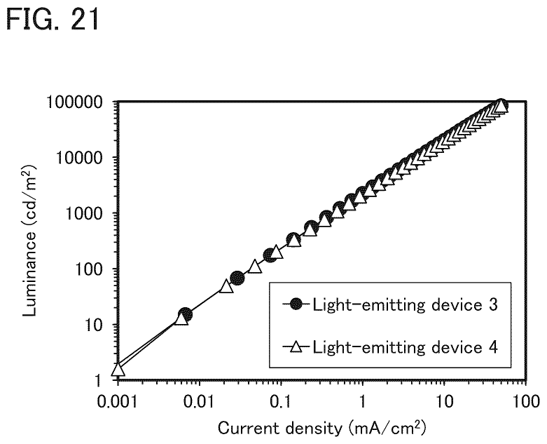

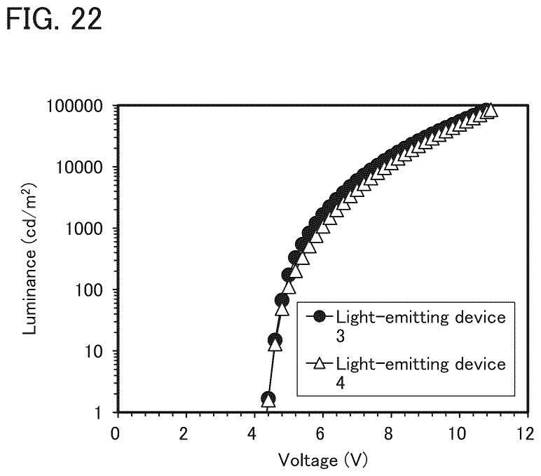

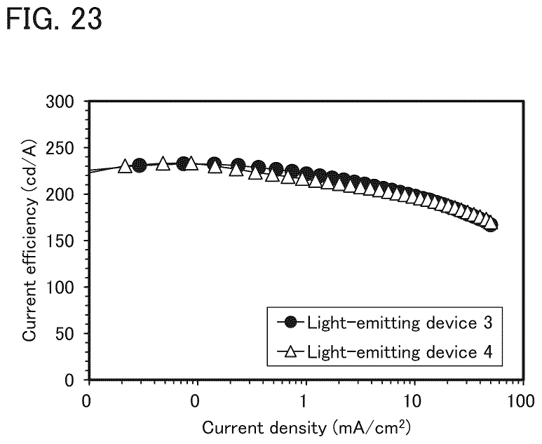

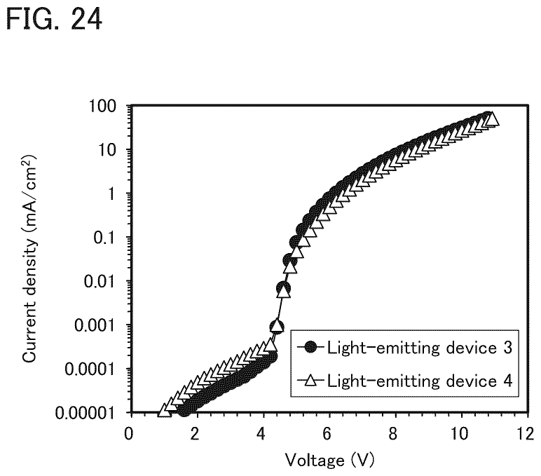

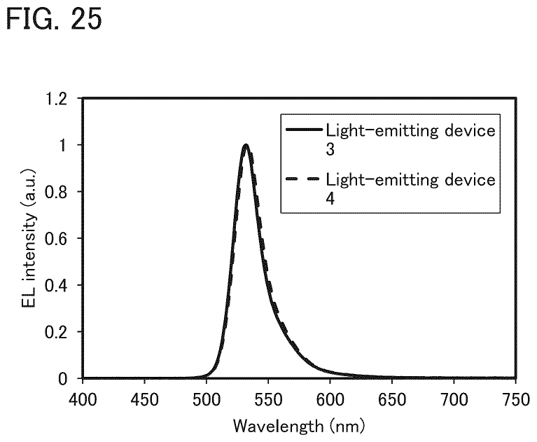

FIG. 21 shows the luminance-current density characteristics of the light-emitting devices 3 and 4, FIG. 22 shows the luminance-voltage characteristics thereof, FIG. 23 shows the current efficiency-current density characteristics thereof, FIG. 24 shows the current density-voltage characteristics thereof, and FIG. 25 shows the electroluminescence spectra thereof.

The table below shows the main characteristics of the light-emitting devices 3 and 4 at a luminance of approximately 1000 cd/m². The luminance, CIE chromaticity, and electroluminescence spectra were measured at normal temperature with a spectroradiometer (SR-ULIR manufactured by TOPCON TECHNOHOUSE CORPORATION).

TABLE 4

| | | | Current | | | | Current |
|---|---|---|---|---|---|---|---|
| | Voltage | Current | density | | | Luminance | efficiency |
| | (V) | (mA) | (mA/cm²) | Chromaticity x | Chromaticity y | (cd/m²) | (cd/A) |
| Light-emitting device 3 | 5.6 | 0.015 | 0.36 | 0.25 | 0.72 | 830 | 228 |
| Light-emitting device 4 | 6.0 | 0.019 | 0.49 | 0.26 | 0.71 | 1076 | 221 |

FIGS. 21 to 25 and the above table reveal that the light-emitting devices 3 and 4 exhibit green light emission derived from Ir(5mppy-d₃)₂(mbfpypy-d₃) and have tandem structures with favorable emission characteristics.

It was thus found that a light-emitting device can be fabricated by a process in which the organic compound layer is formed through processing by a photolithography method using the organic compound of one embodiment of the present invention. It was also found that a tandem light-emitting device with a low driving voltage and high emission efficiency can be fabricated using the organic compound of one embodiment of the present invention.

### Example 4

In this example, the comparison results of the external shapes of the light-emitting devices 3 and 4 described in Example 3 and a comparative light-emitting device 5 are described.

### (Method for Fabricating Comparative Light-Emitting Device 5)

The comparative light-emitting device 5 is different from the light-emitting device 3 in the structure of the first layer of the intermediate layer. That is, the fabrication method of the comparative light-emitting device 5 is different from that of the light-emitting device 3 in that after the formation of the first electron-transport layer, 4,7-di-1-pyrrolidinyl-1,10-phenanthroline (abbreviation: Pyrrd-Phen), which is the comparative organic compound, and indium (In) were deposited by co-evaporation to a thickness of 5 nm such that the volume ratio of Pyrrd-Phen to In was 1:0.2, whereby the first layer of the intermediate layer was formed. The structural formula of Pyrrd-Phen is shown below,

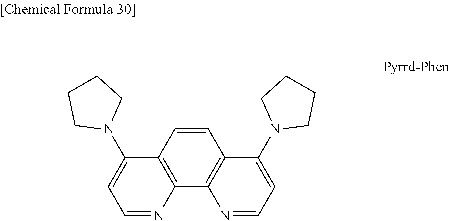 [SMILES: c1cc(N2CCCC2)c2ccc3c(N4CCCC4)ccnc3c2n1]

The comparative light-emitting device 5 was fabricated in a manner similar to that of the light-emitting device 3 except that the thicknesses of the first and second hole-transport layers were 135 nm and 60 nm, respectively, that the second sacrificial layer was processed using an etching gas containing SF₆ and O₂ after the processing by a photolithography method, that the first sacrificial layer was removed using a basic chemical solution after the removal of the second sacrificial layer, and that the heat treatment was performed at 110° C. after the top surface of the second electron-transport layer was exposed.

The table below lists the structure of the comparative light-emitting device 5.

TABLE 5

| | | Thickness | | Comparative light-emitting device 5 |
|---|---|---|---|---|
| Cap layer | | 70 | nm | DBT3P-II |
| Second electrode | | 15 | nm | Ag:Mg (1:0.1) |
| Electron-injection layer | | 1.5 | nm | LiF:Yb (1:0.5) |
| — | | | | Processed by photolithography method |
| Second electron- | 2 | 20 | nm | mPPhen2P |
| transport layer | 1 | 20 | nm | 2mPCCzPDBq |
| Second light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| Second hole-transport layer | | 60 | nm | PCBBiF |
| Intermediate layer | Second layer | 10 | nm | PCBBiF:OCHD-003 (1:0.15) |
| | Third layer | 2 | nm | CuPc |
| | First layer | 5 | nm | Pyrryd-Phen:In (1:0.2) |
| First electron-transport layer | | 10 | nm | 2mPCCzPDBq |
| First light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| First hole-transport layer | | 135 | nm | PCBBiF |
| Hole-injection layer | | 10 | nm | PCBBiF:OCHD-003 (1:0.03) |
| First electrode | 2 | 50 | nm | ITSO |
| | 1 | 100 | nm | APC |

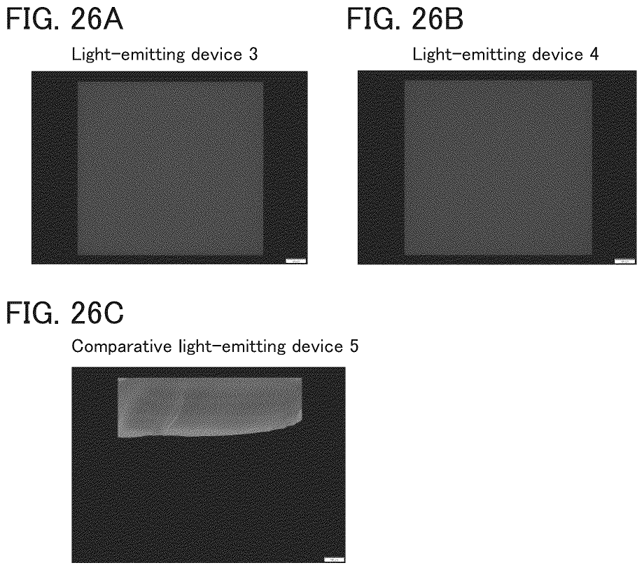

Optical micrographs of the light-emitting devices 3 and 4 and the comparative light-emitting device 5 were taken. The optical micrographs were taken while each light-emitting device having a size of 2 mm×2 mm emitted light by a current of 0.1 mA flowing therethrough, and the light exposure time was 2 ms. FIGS. 26A, 26B, and 26C show optical micrographs of the light-emitting device 3, the light-emitting device 4, and the comparative light-emitting device 5, respectively.

It was found from FIG. 26C that a luminous defect was generated in the comparative light-emitting device 5. This luminous defect occurred in the comparative light-emitting device 5 because it underwent a process in which the organic compound layer was formed through processing by a photolithography method and the quality of the organic compound layer thereby degraded.

Meanwhile, it was found from FIGS. 26A and 26B that a luminous defect occurred in neither the light-emitting device 3 nor the light-emitting device 4 that underwent the same process in which the organic, compound layer was formed through processing by a photolithography method like the comparative light-emitting device 5.

Here, T_{g} of Pyrrd-Phen, which is the comparative organic compound used for the comparative light-emitting device 5, was measured and subjected to a solubility test, and the results were compared to that of PrdP2Phen used for the light-emitting devices 3 and 4.

Note that T_{g} of Pyrrd-Phen was measured with a differential scanning calorimeter (DSC8500, manufactured by PerkinElmer Japan Co., Ltd.) in a state where a powder was put on an aluminum cell and the temperature was increased at a rate of 40° C./min. As a result, T_{g} of Pyrrd-Phen was 78° C.

As described in Example 1, T_{g} of PrdP2Phen used in each of the light-emitting devices 3 and 4 is 130° C., which shows that PrdP2Phen is an organic compound whose T_{g} is higher than that of Pyrrd-Phen.

### <Solubility Test of Pyrrd-Phen by LC-MS Analysis>

In the LC-MS analysis, liquid chromatography (LC) separation was carried out with ACQUITY UPLC manufactured by Waters Corporation, and MS analysis (mass spectrometry) was carried out with Xevo G2 Tof MS manufactured by Waters Corporation. Acquity UPLC BEH C8 (2.1×100 mm, 1.7 μm) was used as a column for the LC separation. Acetonitrile was used for Mobile Phase A and a 0.1% aqueous solution of formic acid was used for Mobile Phase B. The injection amount of the sample was 5.0 μL. Note that in the analysis, the wavelength of a photodiode array detector was set to 263 nm±1 nm.

In a 5-mL sample bottle, 1 mg of Pyrrd-Phen was put and 2 mL of toluene was added thereto. This mixture was irradiated with ultrasonic waves for five minutes. After it was confirmed that the solid was completely dissolved, this solution was diluted by 5 times with acetonitrile, whereby the concentration of the solution was adjusted to 100 mg/L. This solution was diluted with a 3:7 toluene-acetonitrile mixed solvent, whereby a solution with a concentration of 20 mg/L, a solution with a concentration of 10 mg/L, and a solution with a concentration of 5 mg/L were prepared. The prepared solutions were subjected to LC-MS analysis, and the peak area values derived from Pyrrd-Phen, which were obtained at the respective solution concentrations, were used to form calibration curves.

Next, the water solubility of Pyrrd-Phen was measured.

In a 5-mL sample bottle, 1 mg of Pyrrd-Phen was put and 1 mL of water was added thereto. This mixture was irradiated with ultrasonic waves for five minutes. This mixture was filtered through a membrane filter to remove the solid, and the resulting filtrate was diluted by five times with acetonitrile. The obtained solution was subjected to LC-MS analysis.

From the calibration curve and the signal intensity obtained by the LC-MS analysis, it was found that the solubility of Pyrrd-Phen in 1 mL of water is 0.059 mg. The weight fraction of the water solubility of Pyrrd-Phen is therefore 5.9×10⁻⁵.

The mixture of PrdP2Phen and water used in each of the light-emitting devices 3 and 4 was subjected to LC-MS analysis in a manner similar to that described in Example 1, and as a result, the peak area value derived from PrdP2Phen failed to be obtained.

The above results show that Pyrrd-Phen in which a phenylene group is not included between a phenanthroline group and a pyrrolidine group has a low T_{g} and high water solubility.

Thus, it can be said that the luminous defect occurred in the comparative light-emitting device 5 because it underwent a process in which the organic compound layer was formed through processing by a photolithography method using Pyrrd-Phen with a low T_{g} and high water solubility and the quality of the organic compound layer thereby degraded (see FIG. 26C). Meanwhile, even though the light-emitting devices 3 and 4 each underwent a process in which the organic compound layer was formed through processing by a photolithography method, as compared with the comparative light-emitting device 5, the qualities of the organic compound layers are less likely to be degraded, so that luminance defects are inhibited owing to the use of PrdP2Phen (FIGS. 26A and 26B).

The above results show that the use of the organic compound of one embodiment of the present invention enables a light-emitting device having favorable characteristics in which a luminance defect is inhibited to be fabricated even when the fabrication process includes processing using water or a chemical solution containing water as a solvent (i.e., involving processing by a lithography method).

### Example 5

### Synthesis Example 2

In this example, a method for synthesizing 4-(2,3,3a,4,5,6,7,7a-octahydro-1H-isoindol-2-yl)-7-[4-(I-pyrrolidinyl)phenyl]-1,10-phenanthroline (abbreviation: Hid-PrdPPhen) represented by Structural Formula (152) in Embodiment 1 is described. The structural formula of Hid-PrdPPhen is shown below.

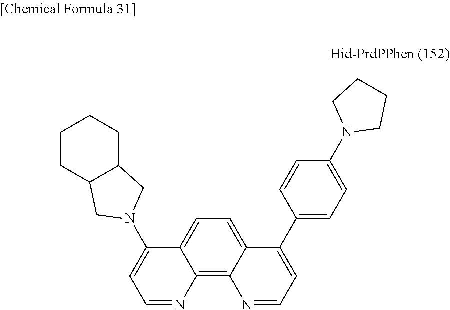 [SMILES: c1cc(-c2ccc(N3CCCC3)cc2)c2ccc3c(N4CC5CCCCC5C4)ccnc3c2n1]

### Step I: synthesis of 4-chloro-7-(2,3,3a,4,5,6,7,7a-octahydro-1H-isoindol-2-yl)-1,10-phenanthroline

In a 1000 mL three-neck flask were put 29.9 g (120 mmol) of 4,7-dichloro-1,10-phenanthroline, 15.0 g (120 mmol) of 2,3,3a,4,5,6,7,7a-octahydro-1H-isoindole, 49.8 g (360 mmol) of potassium carbonate, and 240 mL of 1-methyl-2-pyrrolidone (NMP), and the mixture was stirred at 100° C. under a nitrogen stream for 4 hours. After the stirring, the mixture was cooled down to room temperature. Impurities of this mixture were separated by suction filtration, and the obtained filtrate was subjected to extraction with chloroform. The extracted solution was concentrated to give a mixture of a solid and an oily substance. This solid was collected by suction filtration. Ethyl acetate was added to this solid and the solid was collected by suction filtration, whereby 33.5 g of a target pale yellow solid was obtained in a yield of 82.7%. The synthesis scheme of 4-chloro-7-(2,3,3a,4,5,6,7;7a-octahydro-11-isoindol-2-yl)-1,10-phenanthroline is shown in Formula (b-1) below.

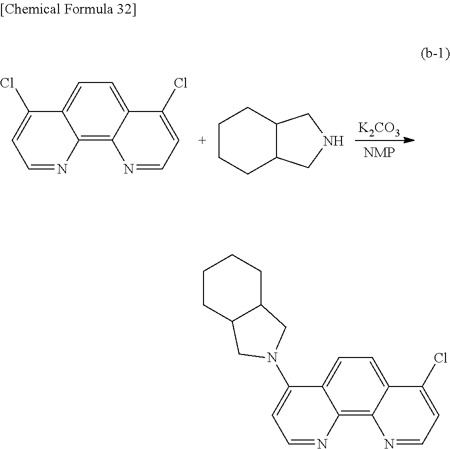

### Step 2: Synthesis of Hid-PrdPPhen

In a 300 mL three-neck flask were put 3.60 g (10.7 mmol) of 4-chloro-7-(2,3,3a,4,5,6,7,7a-octahydro-1H-isoindol-2-yl)-1,10-phenanthroline, 2.45 g (12.8 mmol) of 4-(1-pyrrolidinyl)phenylboronic acid, 0.105 g (0.374 mmol) of tricyclohexylphosphine (approximately 18% in toluene solution), 4.07 g (19.2 mmol) of tripotassium phosphate, 63 mL of 1,4-dioxane, and 31 mL of water, and the mixture was degassed by being stirred under reduced pressure. To this mixture was added 0.146 g (0.159 mmol) of tris(dibenzylideneacetone)dipalladium(0) and stirring was performed at 100° C. under a nitrogen stream for 24 hours. After the stirring, the mixture was cooled down to room temperature. This mixture was subjected to extraction with chloroform. The extracted solution was concentrated to give an oily substance. The oily substance was purified by silica gel column chromatography (developing solvent: methanol). The obtained fraction was concentrated to give a solid. Chloroform was added to this solid to dissolve the solid, a 3-mercaptopropyl silica gel was added thereto, and stirring was performed at room temperature for 1 hour. After the stirring, an insoluble matter was separated by gravity filtration, and the obtained filtrate was concentrated to give a solid. Ethyl acetate and hexane was added to this solid, irradiation with ultrasonic waves was performed, and the solid was separated by suction filtration to give 0.810 g of a target yellow solid in 16.9% yield. The synthesis scheme of Hid-PrdPPhen is shown in Formula (b-2) below,

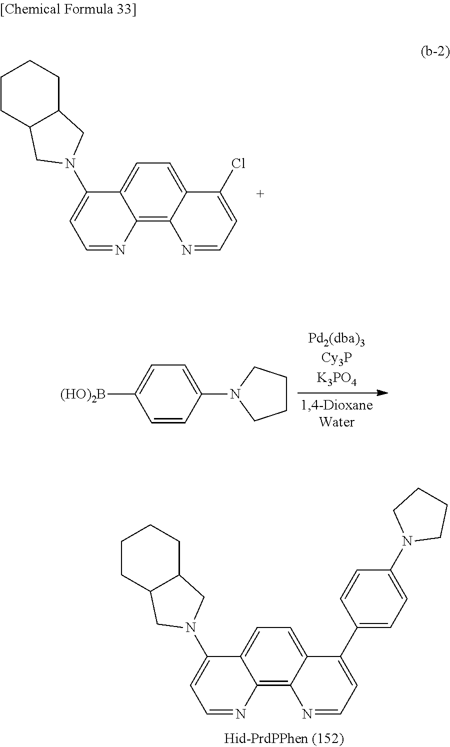

By a train sublimation method, 0.810 g of the obtained yellow solid was purified by sublimation. In the purification by sublimation, heating was performed for 48 hours at an argon flow rate of 5.0 mL/min, a pressure of 3.0 Pa, and a heating temperature of 240° C. As a result, 0.475 g of a target yellow solid was obtained at a collection rate of 58.6%.

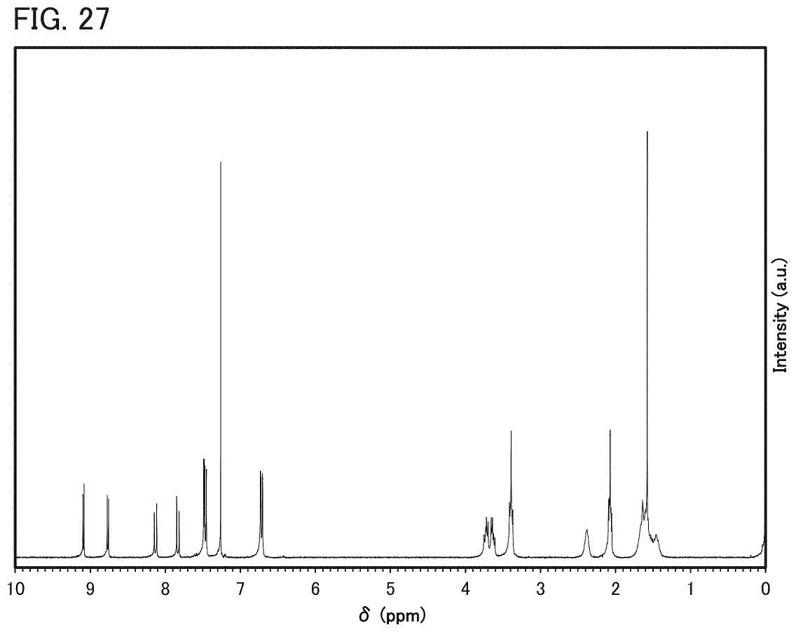

FIG. 27 shows a ¹H NMR spectrum of Hid-PrdPPhen after the purification by sublimation. Results of 1H NMR measurement are shown below. The results show that Hid-PrdPPhen was obtained.

¹H NMR (CDCl₃, 300 MHz): δ=9.09 (d, J=4.8 Hz, 1H), 8.77 (d, J=5.5 Hz, 1H), 8.13 (d, J=9.5 Hz, 1H), 7.83 (d, J=9.5 Hz, 1H), 7.49-7.45 (m, 3H), 6.73-6.70 (m, 3H), 3.75-3.61 (m, 4H), 3.41-3.37 (m, 4H), 2.38 (s, 2H), 2.09-2.05 (m, 4H), 1.67-1.46 (in, 8H).

### <T_{g} Measurement of Hid-PrdPPhen>

Note that T_{g} of Hid-PrdPPhen was measured with a differential scanning calorimeter (DSC8500, manufactured by PerkinElmer Japan Co., Ltd.) in a state where a powder was put on an aluminum cell and the temperature was increased at a rate of 40° C./min. As a result, T_{g} of Hid-PrdPPhen was 115° C.

Next, a solubility test of Hid-PrdPPhen was performed. Note that the solubility test was conducted at a pressure of one atmosphere at room temperature (RT).

### <Solubility Test of Hid-PrdPPhen by LC-MS Analysis>

In the LC-MS analysis, liquid chromatography (LC) separation was carried out with ACQUITY UPLC manufactured by Waters Corporation, and MS analysis (mass spectrometry) was carried out with Xevo G2 Tof MS manufactured by Waters Corporation. Acquity UPLC BEH C8 (2.1×100 mm, 1.7 μm) was used as a column for the LC separation. Acetonitrile was used for Mobile Phase A and a 0.1% aqueous solution of formic acid was used for Mobile Phase B. The injection amount of the sample was 5.0 μL, Note that in the analysis, the wavelength of a photodiode array detector was set to 263 nm±1 nm.

In a 5-mL sample bottle, I mg of Hid-PrdPPhen was put and 1 mL of water was added thereto. This mixture was irradiated with ultrasonic waves for five minutes. This mixture was filtered through a membrane filter to remove the solid, and the resulting filtrate was diluted by five times with acetonitrile. The obtained solution was subjected to LC-MS analysis.

As a result, the peak area value derived from Hid-PrdPPhen failed to be obtained through the LC-MS analysis.

It is thus found that the organic compound of one embodiment of the present invention can be favorably used for a light-emitting device whose fabrication process includes processing using water or a chemical solution containing water as a solvent (i.e., a light-emitting device involving processing by a lithography method).

### Example 6

In this example, a light-emitting device 6 and a light-emitting device 7 each using Hid-PrdPPhen (Structural Formula (152)), which is the organic compound of one embodiment of the present invention, were fabricated and the measurement results of the device characteristics are described. The light-emitting device 6 was fabricated by a fabrication method (what is called a continuous vacuum process) that includes neither a step of exposing the organic compound layer nor a process of forming an organic compound layer through processing by a photolithography method. The light-emitting device 7 was fabricated by a process in which an organic compound layer is formed through processing by a photolithography method. The structural formulae of the organic compounds used in the light-emitting devices 6 and 7 are shown below.

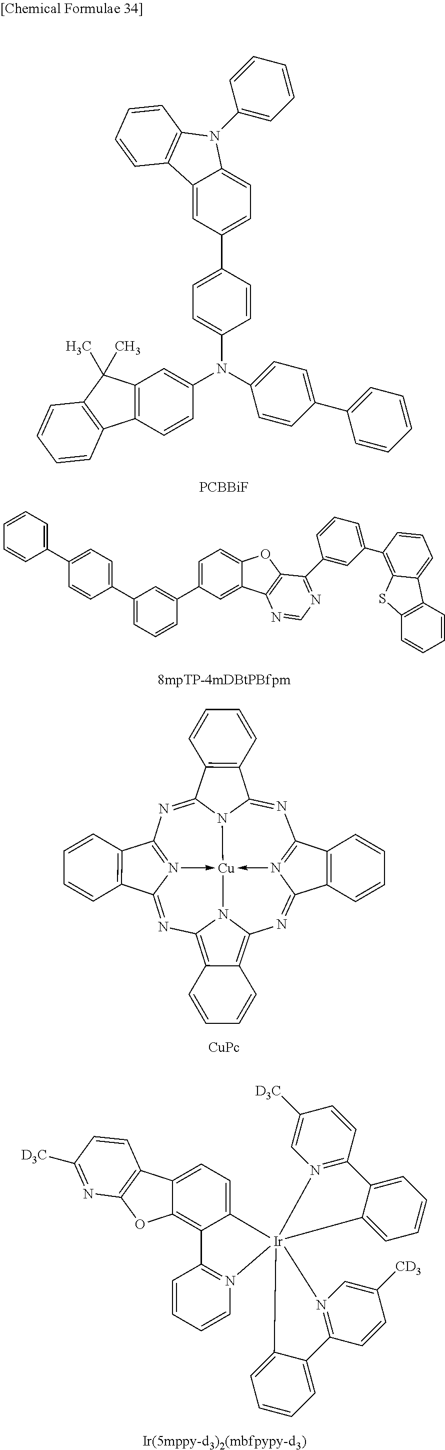

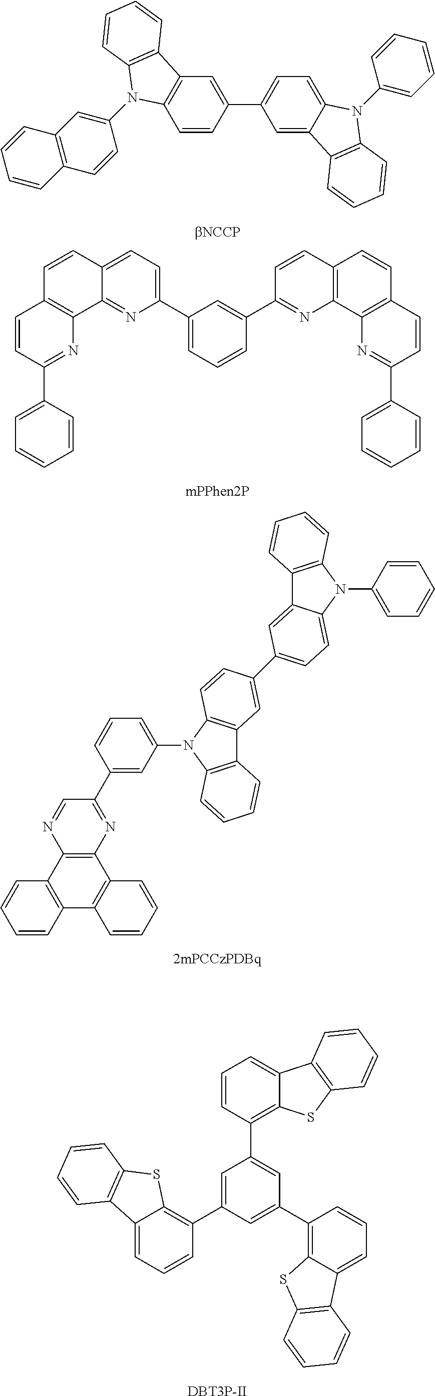

### (Method for Fabricating Light-Emitting Device 6)

First, as a reflective electrode, APC was deposited over a glass substrate to a thickness of 100 nm by a sputtering method, and then, as a transparent electrode, ITSO was deposited to a thickness of 50 nm by a sputtering method, so that the first electrode was formed. The electrode area was set to 4 mm² (2 mm×2 mm). Note that the transparent electrode functions as the anode, and the transparent electrode and the reflective electrode can be collectively regarded as the first electrode.

Next, in pretreatment for forming the light-emitting device over the substrate, the surface of the substrate was washed with water, and baking was performed at 200° C. for 1 hour.

After that, the substrate was transferred into a vacuum evaporation apparatus where the pressure had been reduced to approximately 1×10⁻⁴ Pa, and was subjected to heat treatment at 170° C. for 30 minutes in a heating chamber of the vacuum evaporation apparatus, and then the substrate was cooled down for approximately 30 minutes.

Then, the hole-injection layer was formed. The structure of the hole-injection layer is similar to that of the light-emitting device 1.

Over the hole-injection layer, PCBBiF was deposited by evaporation to a thickness of 110 nm, so that the first hole-transport layer was formed.

Next, the first light-emitting layer was formed over the first hole-transport layer, and then the first electron-transport layer was formed over the first light-emitting layer. The structures of the first light-emitting layer and the first electron-transport layer are similar to those of the light-emitting device 1.

After the formation of the first electron-transport layer, Hid-PrdPPhen, which is the organic compound of one embodiment of the present invention, and indium (In) were deposited by co-evaporation to a thickness of 5 nm such that the volume ratio of Hid-PrdPPhen to In was 1.0:0.02, whereby the first layer of the intermediate layer was formed.

Next, the third layer of the intermediate layer was formed, and then the second layer of the intermediate layer was formed. The structures of the third and second layers of the intermediate layer are similar to those of the light-emitting device 1.

Next, over the intermediate layer, PCBBiF was deposited by evaporation to a thickness of 50 nm, so that the second hole-transport layer was formed.

The second light-emitting layer was formed over the second hole-transport layer, and then the second electron-transport layer was formed. The structures of the second light-emitting layer and the second electron-transport layer are similar to those of the light-emitting device 1.

After the formation of the second electron-transport layer, the electron-injection layer was formed over the second electron-transport layer, and lastly the second electrode was formed, whereby the light-emitting device 3 was fabricated. The cap layer was formed over the second electrode. The structures of the electron-injection layer, the second electrode, and the cap layer are similar to those of the light-emitting device 1.

### (Method for Fabricating Light-Emitting Device 7)

The light-emitting device 7 was fabricated in a manner similar to that of the light-emitting device 6 except that the processing by a photolithography method and the heat treatment were performed after the formation of the second electron-transport layer and before the formation of the electron-injection layer. The processing by a photolithography method and the heat treatment were performed in a manner similar to that of the light-emitting device 3.

The table below lists the structures of the light-emitting devices 6 and 7.

TABLE 6

| | Thickness | | Light-emitting device 6 Light-emitting device 7 |
|---|---|---|---|
| Cap layer | 70 | nm | DBT3P-II |
| Second electrode | 15 | nm | Ag:Mg (1:0.1) |
| Electron-injection layer | 1.5 | nm | LiF:Yb (1:0.5) |

| | | |
|---|---|---|
| Step of photolithography | x | ∘ |

| Second electron- | 2 | 20 | nm | mPPhen2P |
|---|---|---|---|---|
| transport layer | 1 | 20 | nm | 2mPCCzPDBq |
| Second light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| Second hole-transport layer | | 50 | nm | PCBBiF |
| Intermediate | Second layer | 10 | nm | PCBBiF:OCHD-003 (1:0.15) |
| layer | Third layer | 2 | nm | CuPc |
| | First layer | 5 | nm | Hid-PrdPPhen:In |
| | | | | (1:0.02) |
| First electron-transport layer | | 10 | nm | 2mPCCzPDBq |
| First light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| First hole-transport layer | | 110 | nm | PCBBiF |
| Hole-injection layer | | 10 | nm | PCBBiF:OCHD-003 (1:0.03) |
| First electrode | 2 | 50 | nm | ITSO |
| | 1 | 100 | nm | APC |

x: No processing was performed by photolithography method.
∘: Processing was performed by photolithography method.

The light-emitting devices fabricated were each sealed using a glass substrate in a glove box containing a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for 1 hour. Then, the initial characteristics of the light-emitting devices were measured.

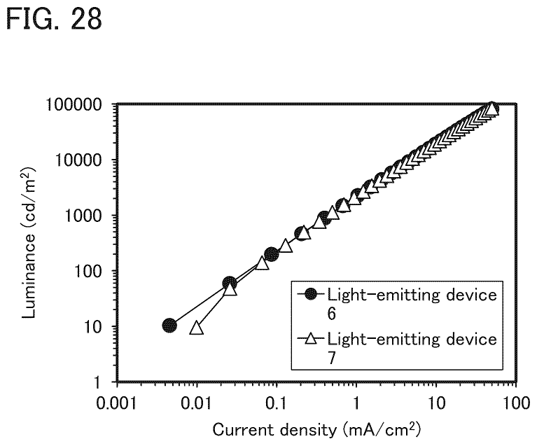

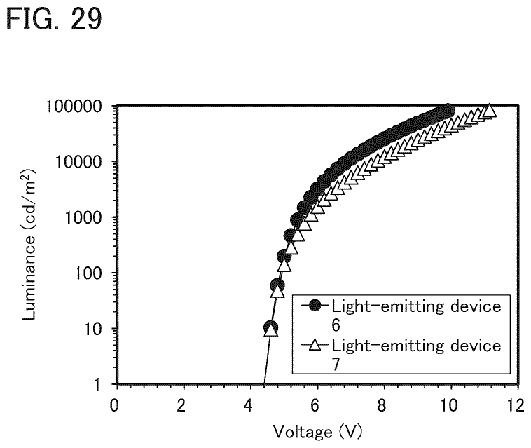

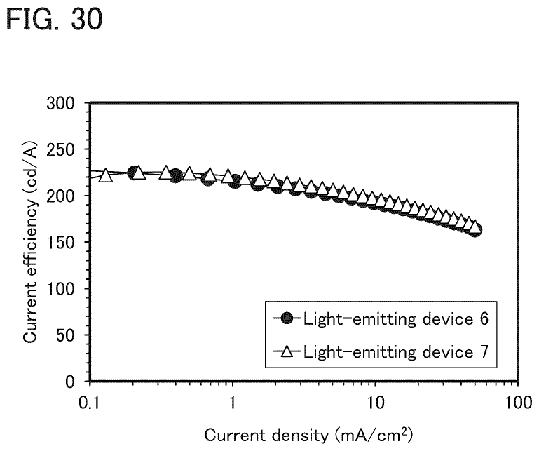

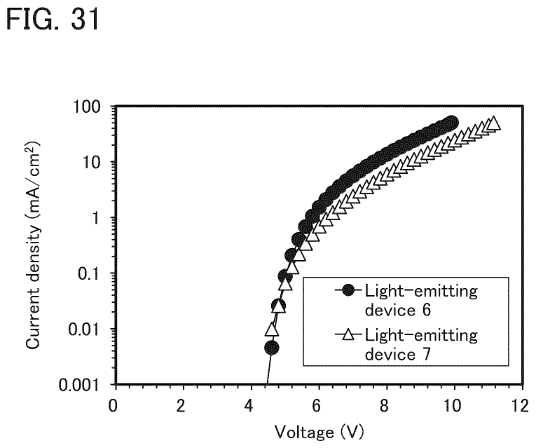

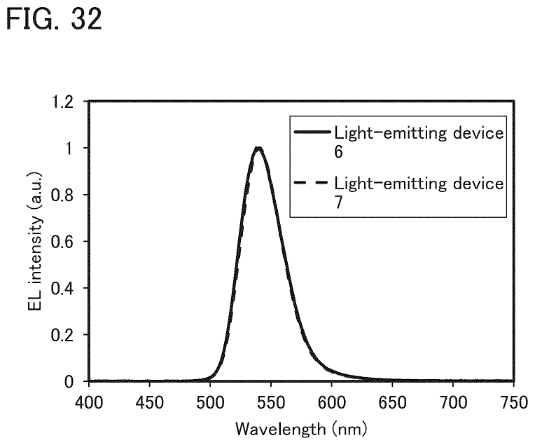

FIG. 28 shows the luminance-current density characteristics of the light-emitting devices 6 and 7, FIG. 29 shows the luminance-voltage characteristics thereof, FIG. 30 shows the current efficiency-current density characteristics thereof, FIG. 31 shows the current density-voltage characteristics thereof, and FIG. 32 shows the electroluminescence spectra thereof.

The table below shows the main characteristics of the light-emitting devices 6 and 7 at a luminance of approximately 1000 cd/m². The luminance, CIE chromaticity, and electroluminescence spectra were measured at normal temperature with a spectroradiometer (SR-ULIR manufactured by TOPCON TECHNOHOUSE CORPORATION).

TABLE 7

| | | | Current | | | | Current |
|---|---|---|---|---|---|---|---|
| | Voltage | Current | density | | | Luminance | efficiency |
| | (V) | (mA) | (mA/cm²) | Chromaticity x | Chromaticity y | (cd/m²) | (cd/A) |
| Light-emitting device 6 | 5.4 | 0.016 | 0.40 | 0.30 | 0.68 | 883 | 221 |
| Light-emitting device 7 | 5.8 | 0.020 | 0.50 | 0.30 | 0.68 | 1119 | 224 |

FIGS. 28 to 37 and the above table reveal that the light-emitting devices 6 and 7 exhibit green light emission derived from Ir(5mppy-d₃)₂(mbfpypy-d₃) and have tandem structures with favorable emission characteristics.

It was thus found that a light-emitting device having favorable characteristics can be fabricated by either a continuous vacuum process or a process in which the organic compound layer is formed through processing by a photolithography method using the organic compound of one embodiment of the present invention. It was also found that a tandem light-emitting device with a low driving voltage and high emission efficiency can be fabricated using the organic compound of one embodiment of the present invention.

### Example 7

In this example, a single-type light-emitting device 8 using PrdP2Phen (Structural Formula (100)), which is the organic compound of one embodiment of the present invention, for the electron-injection layer and a single-type comparative light-emitting device 9 using a comparative organic compound for the electron-injection layer were fabricated, and the comparison results of the device characteristics are described. Furthermore, in this example, the comparison results of the external shapes of the light-emitting device 8 and the comparative light-emitting device 9 are described. The light-emitting device 8 and the comparative light-emitting device 9 were each fabricated by a method that includes a process in which an organic compound layer is formed through processing by a photolithography method after the formation of the electron-injection layer. The structural formulae of the organic compounds used in the light-emitting device 8 and the comparative light-emitting device 9 are shown below.

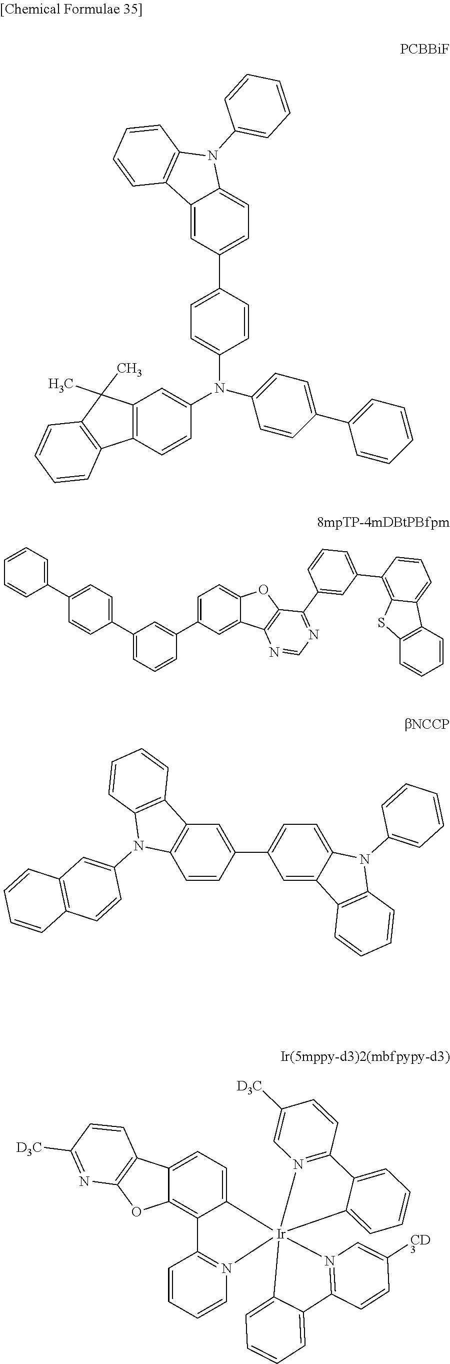

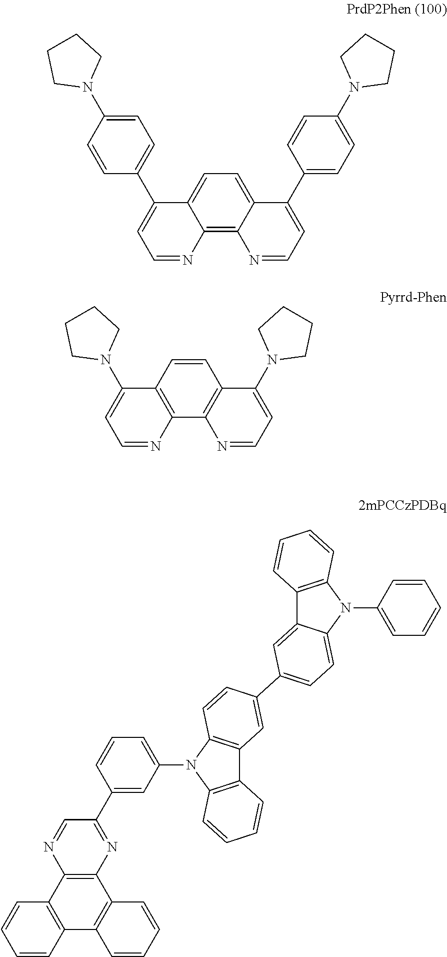

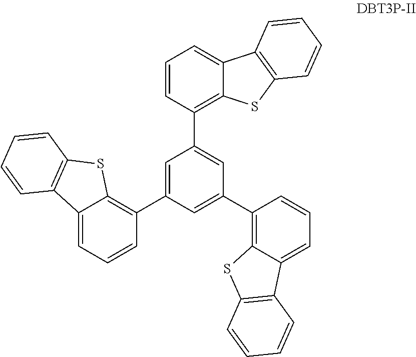 [SMILES: c1ccc2c(c1)sc1c(-c3cc(-c4cccc5c4sc4ccccc45)cc(-c4cccc5c4sc4ccccc45)c3)cccc12]

### (Method for Fabricating Light-Emitting Device 8)

First, as a reflective electrode, APC was deposited over a glass substrate to a thickness of 100 nm by a sputtering method, and then, as a transparent electrode, ITSO was deposited to a thickness of 50 nm by a sputtering method, so that the first electrode was formed. The electrode area was set to 4 mm² (2 mm×2 mm). Note that the transparent electrode functions as the anode, and the transparent electrode and the reflective electrode can be collectively regarded as the first electrode.

Next, in pretreatment for forming the light-emitting device over the substrate, the surface of the substrate was washed with water, and baking was performed at 200° C. for 1 hour.

After that, the substrate was transferred into a vacuum evaporation apparatus where the pressure had been reduced to approximately 1×10⁻⁴ Pa, and was subjected to heat treatment at 170° C. for 30 minutes in a heating chamber of the vacuum evaporation apparatus, and then the substrate was cooled down for approximately 30 minutes.

Then, hole-injection layer was formed. The structure of the hole-injection layer is similar to that of the light-emitting device 1.

Over the hole-injection layer, PCBBiF was deposited by evaporation to a thickness of 100 nm, so that a hole-transport layer was formed.

Next, a light-emitting layer was formed over the hole-transport layer. The structure of the light-emitting layer is similar to that of the light-emitting device 1.

Then, 2mPCCzPDBq was deposited by evaporation to a thickness of 20 nm, so that an electron-transport layer was formed.

After the formation of the electron-transport layer, PrdP2Phen, which is the organic compound of one embodiment of the present invention, was deposited by evaporation to a thickness of 5 nm, so that the electron-injection layer was formed.

After the formation of the electron-injection layer, the processing by a photolithography method and the heat treatment were performed. The processing by a photolithography method and heat treatment were performed in a manner similar to that of the light-emitting device 3.

After the processing by a photolithography method and the heat treatment, the second electrode was formed over the top surface of the exposed organic compound layer, i.e., over the electron-injection layer, whereby the light-emitting device 8 was fabricated. The cap layer was formed over the second electrode. The structures of the electron-injection layer, the second electrode, and the cap layer are similar to those of the light-emitting device 1.

### (Method for Fabricating Comparative Light-Emitting Device 9)

The comparative light-emitting device 9 is different from the light-emitting device 8 in that PrdP2Phen used for the electron-injection layer of the light-emitting device 8 was replaced with Pyrrd-Phen, which is a comparative organic compound. The other components were formed in the same manner as those in the light-emitting device 8.

The table below lists the structures of the light-emitting device 8 and the comparative light-emitting device 9.

TABLE 8

| | | | Light-emitting | Comparative light- |
|---|---|---|---|---|
| | Thickness | | device 8 | emitting device 9 |
| Cap layer | 70 | nm | DBT3P-II | |
| Second electrode | 15 | nm | Ag:Mg (1:0.1) | |
| — | | | Processed by photolithography method | |
| Electron-injection layer | 5 | nm | PrdP2Phen | Pyrrd-Phen |

| Electron-transport layer | | 20 | nm | 2mPCCzPDBq |
|---|---|---|---|---|
| Light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| Hole-transport layer | | 100 | nm | PCBBiF |
| Hole-injection layer | | 10 | nm | PCBBiF:OCHD-003 (1:0.03) |
| First electrode | 2 | 50 | nm | ITSO |
| | 1 | 100 | nm | APC |

The light-emitting devices fabricated were each sealed using a glass substrate in a glove box containing a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for 1 hour. Then, the initial characteristics of the light-emitting devices were measured.

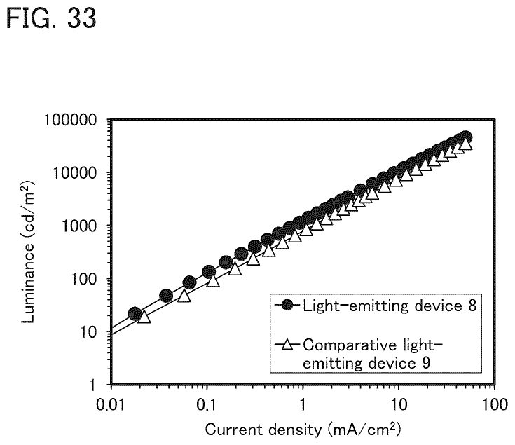

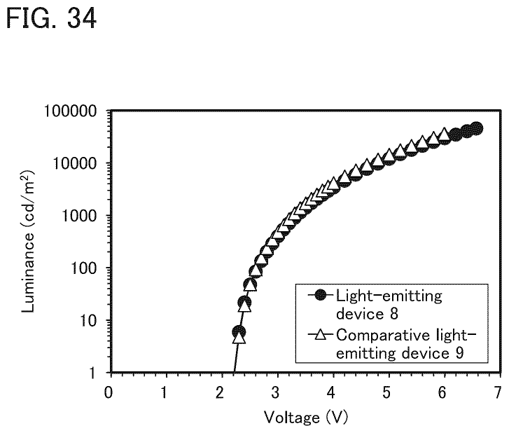

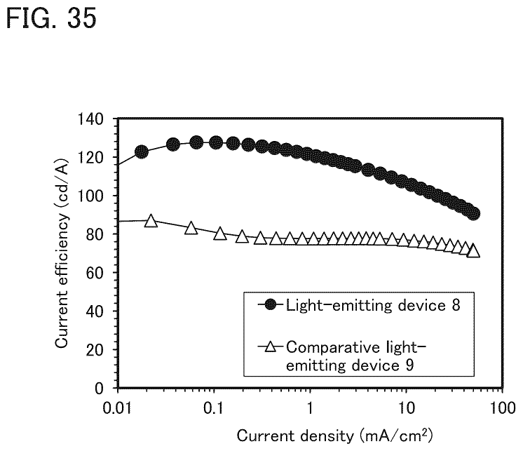

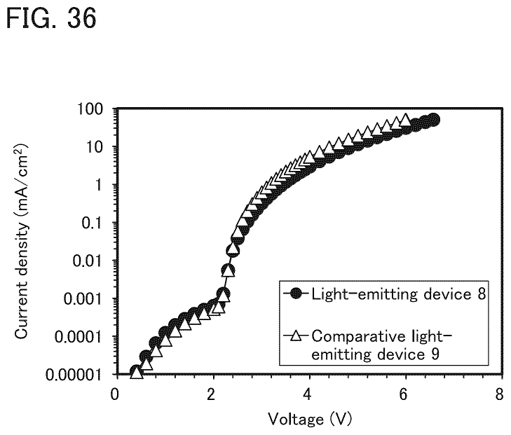

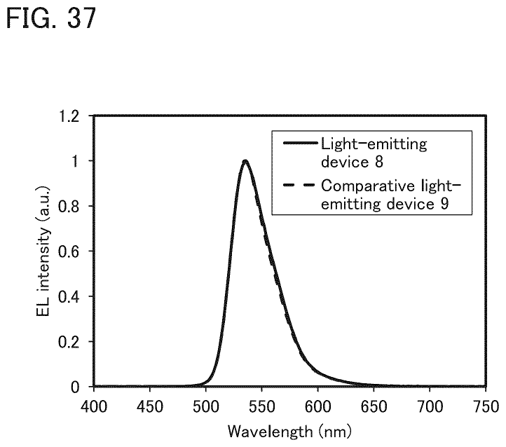

FIG. 33 shows the luminance-current density characteristics of the light-emitting device 8 and the comparative light-emitting device 9, FIG. 34 shows the luminance-voltage characteristics thereof, FIG. 35 shows the current efficiency-current density characteristics thereof, FIG. 36 shows the current density-voltage characteristics thereof, and FIG. 37 shows the electroluminescence spectra thereof.

The table below shows the main characteristics of the light-emitting device 8 and the comparative light-emitting device 9 at a luminance of approximately 1000 cd/m². The luminance, CIE chromaticity, and electroluminescence spectra were measured at normal temperature with a spectroradiometer (SR-UL1R manufactured by TOPCON TECHNOHOUSE CORPORATION).

TABLE 9

| | | | Current | | | | Current |
|---|---|---|---|---|---|---|---|
| | Voltage | Current | density | | | Luminance | efficiency |
| | (V) | (mA) | (mA/cm²) | Chromaticity x | Chromaticity y | (cd/m²) | (cd/A) |
| Light-emitting device 8 | 3.3 | 0.029 | 0.73 | 0.30 | 0.67 | 893 | 123 |
| Comparative light- | 3.3 | 0.056 | 1.39 | 0.30 | 0.68 | 1083 | 78 |
| emitting device 9 | | | | | | | |

It was found from FIGS. 33 to 37 and the above table that the light-emitting device 8 and the comparative light-emitting device 9 exhibit green light emission derived from Ir(5mppy-d₃)₂(mbfpypy-d₃). It was also found that the light-emitting device 8 has high current efficiency and favorable emission characteristics.

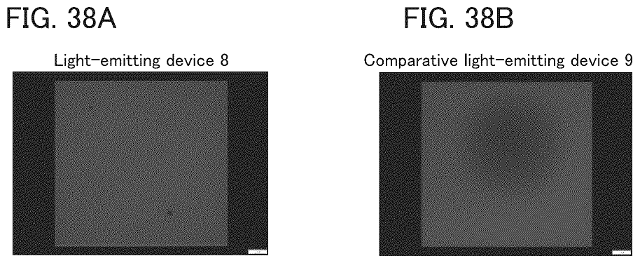

Optical micrographs of the light-emitting device 8 and the comparative light-emitting device 9 were taken. The optical micrographs were taken while each light-emitting device having a size of 2 mm×2 mm emitted light by a current of 0.1 mA flowing therethrough, and the light exposure time was 2 ms. FIGS. 38A and 38B show optical micrographs of the light-emitting device 8 and the comparative light-emitting device 9, respectively.

It was found from FIG. 38B that a luminous defect was generated in the comparative light-emitting device 9. This luminous defect occurred in the comparative light-emitting device 9 because it underwent a process in which the organic compound layer was formed through processing by a photolithography method using Pyrrd-Phen with a low T_{g} and high water solubility and the quality of the organic compound layer thereby degraded.

Meanwhile, it was found from FIG. 38A that a luminous defect did not occur in the light-emitting device 8 that underwent the same process in which the organic compound layer was formed through processing by a photolithography method like the comparative light-emitting device 9.

It was thus found that the use of the organic compound of one embodiment of the present invention enables a light-emitting device having favorable characteristics in which a luminance defect is inhibited to be fabricated even when the fabrication process includes processing using water or a chemical solution containing water as a solvent (i.e., involving processing by a lithography method).

The above results show that a light-emitting device can be fabricated by a process in which the organic compound layer is formed through processing by a photolithography method using the organic compound of one embodiment of the present invention. Furthermore, it was found that a light-emitting device having favorable characteristics can be fabricated using the organic compound of one embodiment of the present invention.

This application is based on Japanese Patent Application Serial No. 2023-158365 filed with Japan Patent Office on Sep. 22, 2023 and 2024-134216 filed with Japan Patent Office on Aug. 9, 2024, the entire contents of which are hereby incorporated by reference.

What is claimed is:

1. An organic compound represented by Formula (G1-1), 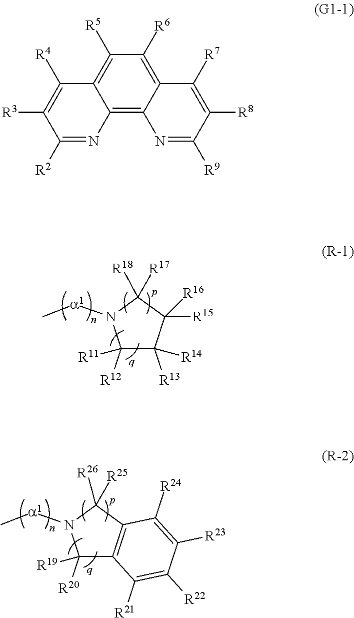
wherein:
at least any one of R² to R⁹ is a group represented by Formula (R-1) or (R-2), and each of the others of R² to R⁹ independently represents any one of hydrogen, an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, and a substituted or unsubstituted heteroaryl group having 1 to 30 carbon atoms;
α¹ represents a substituted or unsubstituted arylene group having 6 to 30 carbon atoms;
n represents 1 or 2:
in the case where n is 2, two arylene groups represented as α¹ are the same or different from each other;
each of R¹¹ to R²⁶ independently represents one of hydrogen and an alkyl group having 1 to 10 carbon atoms; and
each of p and q independently represents 0 or 1.

2. The organic compound according to claim 1, wherein R⁴ or R⁷, or both of R⁴ and R⁷ are a group represented by Formula (R-1) or (R-2).

3. The organic compound according to claim 1, wherein any two of R¹¹ to R¹⁸ are bonded to each other to form a ring.

4. The organic compound according to claim 1, wherein any two of R¹⁹ to R²⁶ are bonded to each other to form a ring.

5. The organic compound according to claim 1,
wherein some of R¹¹ to R²⁶ are hydrogen, and
wherein any one of the hydrogens as R¹¹ to R²⁶ is deuterium.

6. A light-emitting device comprising the organic compound according to claim 1.

7. A light-emitting device comprising:
a first electrode, a second electrode, and an organic compound layer between the first electrode and the second electrode,
wherein the organic compound layer comprises a light-emitting layer and an electron-injection layer, and
wherein the electron-injection layer comprises the organic compound according to claim 1.

8. A light-emitting device comprising:
a first electrode, a second electrode, and an organic compound layer between the first electrode and the second electrode,
wherein the organic compound layer comprises a first light-emitting layer, a second light-emitting layer, and an intermediate layer between the first light-emitting layer and the second light-emitting layer, and
wherein the intermediate layer comprises the organic compound according to claim 1.

9. A light-emitting apparatus comprising:
the light-emitting device according to claim 6; and
one of a transistor and a substrate.

10. An electronic appliance comprising:
the light-emitting apparatus according to claim 9; and
any one of a detection portion, an input portion and a communication portion.

11. An organic compound represented by Formula (G1-2), 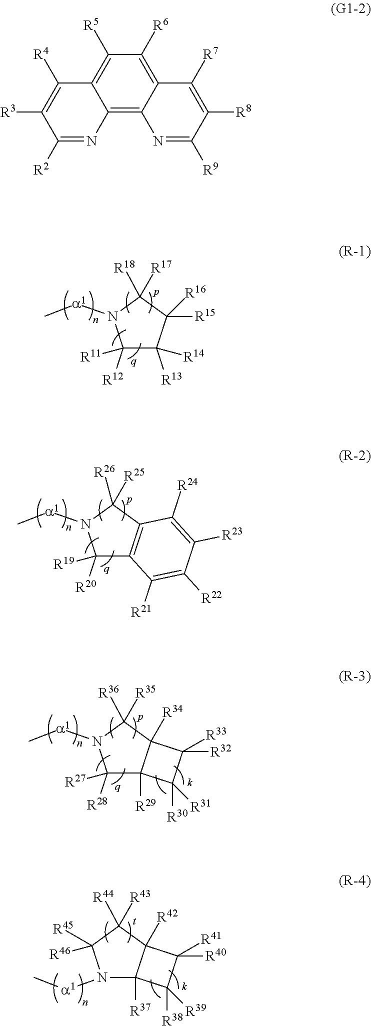
wherein:
at least any one of R² to R⁹ is a group represented by any of Formulae (R-1) to (R-4), and each of the others of R² to R⁹ independently represents any one of hydrogen, an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, and a substituted or unsubstituted heteroaryl group having 1 to 30 carbon atoms;
α¹ represents a substituted or unsubstituted arylene group having 6 to 30 carbon atoms;
n represents 1 or 2;
in the case where n is 2, two arylene groups represented as a, are the same or different from each other;
each of R¹¹ to R⁴⁶ independently represents one of hydrogen and an alkyl group having 1 to 10 carbon atoms;
each of p and q independently represents 0 or 1;
k represents an integer of 0 to 4; and
t represents 0 or 1.

12. The organic compound according to claim 11, wherein R⁴ or R⁷, or both of R⁴ and R⁷ are a group represented by any of Formulae (R-1) to (R-4).

13. The organic compound according to claim 11, wherein any of two of R¹¹ to R¹⁸, two of R¹⁹ to R²⁶, two of R²⁷ to R³⁶, and two of R³⁷ to R⁴⁶ are bonded to each other to form a ring.

14. The organic compound according to claim 11,
wherein:
the organic compound is represented by Formula (G1-3); 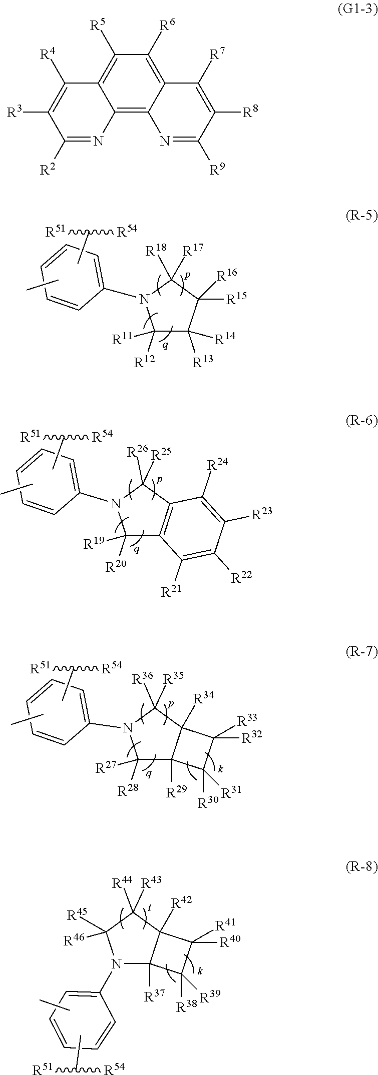
at least any one of R² to R⁹ is a group represented by any of Formulae (R-5) to (R-8), and each of the others of R² to R⁹ independently represents any one of hydrogen, an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, and a substituted or unsubstituted heteroaryl group having 1 to 30 carbon atoms;
each of R⁵¹ to R⁵⁴ independently represents any one of hydrogen, an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, and a substituted or unsubstituted heteroaryl group having 1 to 30 carbon atoms;
each of R¹¹ to R⁴⁶ independently represents one of hydrogen and an alkyl group having 1 to 10 carbon atoms;
each of p and q independently represents 0 or 1;
k represents an integer of 0 to 4; and
t represents 0 or 1.

15. The organic compound according to claim 14, wherein R⁴ or R⁷, or both of R⁴ and R⁷ are a group represented by any of Formulae (R-5) to (R-8).

16. The organic compound according to claim 14, wherein any of two of R¹¹ to R¹⁸, two of R¹⁹ to R²⁶, two of R²⁷ to R³⁶, and two of R³⁷ to R⁴⁶ are bonded to each other to form a ring.

17. The organic compound according to claim 14,
wherein some of R¹¹ to R⁴⁶ are hydrogen, and
wherein any one of the hydrogens as R¹¹ to R⁴⁶ is deuterium.

18. The organic compound according to claim 14,
wherein:
the organic compound is represented by Formula (G1-4); 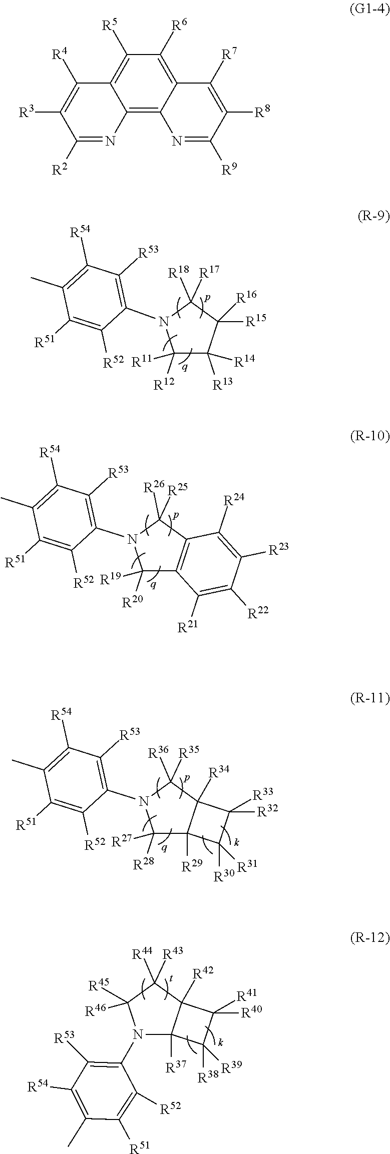
at least any one of R² to R⁹ is a group represented by any of Formulae (R-9) to (R-12), and each of the others of R² to R⁹ independently represents any one of hydrogen, an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, and a substituted or unsubstituted heteroaryl group having 1 to 30 carbon atoms;
each of R⁵¹ to R⁵⁴ independently represents any one of hydrogen, an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, and a substituted or unsubstituted heteroaryl group having 1 to 30 carbon atoms;
each of R¹¹ to R⁴⁶ independently represents one of hydrogen and an alkyl group having 1 to 10 carbon atoms;
p and q each independently represent 0 or 1;
k represents an integer of 0 to 4; and
t represents 0 or 1.

19. The organic compound according to claim 18, wherein R⁴ or R⁷, or both of R⁴ and R⁷ are a group represented by any of Formulae (R-5) to (R-8).

20. The organic compound according to claim 18, wherein any of two of R¹¹ to R¹⁸, two of R¹⁹ to R²⁶, two of R²⁷ to R³⁶, and two of R³⁷ to R⁴⁶ are bonded to each other to form a ring.

21. The organic compound according to claim 18, wherein any one of the others of R² to R⁹ is deuterium.

22. The organic compound according to claim 11,
wherein:
the organic compound is represented by Formula (G2); 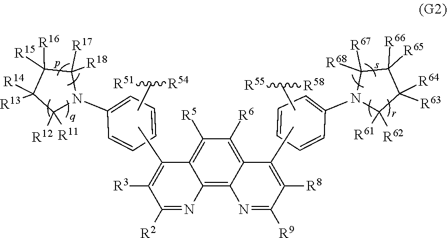
each of R², R³, R⁵, R⁶, R⁸, R⁹, and R⁵¹ to R⁵⁸ independently represents any one of hydrogen, an alkyl group having 1 to 10 carbon atoms, a cycloalkyl group having 3 to 10 carbon atoms, an alkoxy group having 1 to 10 carbon atoms, a substituted or unsubstituted cyclic secondary amino group having 2 to 10 carbon atoms, a substituted or unsubstituted aryl group having 6 to 30 carbon atoms, and a substituted or unsubstituted heteroaryl group having 1 to 30 carbon atoms;
each of R¹¹ to R¹⁸ and R⁶¹ to R⁶⁸ independently represents one of hydrogen and an alkyl group having 1 to 10 carbon atoms; and
each of p, q, r and s independently represents 0 or 1.

23. The organic compound according to claim 22,
wherein the organic compound is represented by Formula (G3):

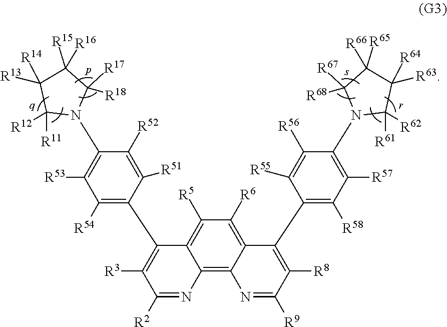

24. The organic compound according to claim 22,
wherein the organic compound is represented by Formula (G4):

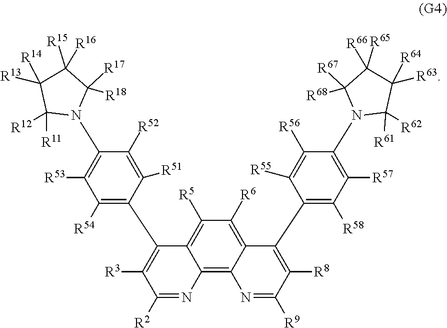

25. The organic compound according to claim 22, wherein a group at 4-position and a group at 7-position of the phenanthroline ring in Formula (G2) are the same.

26. The organic compound according to claim 22,
wherein the organic compound is represented by one of Formulae (100) and (152):

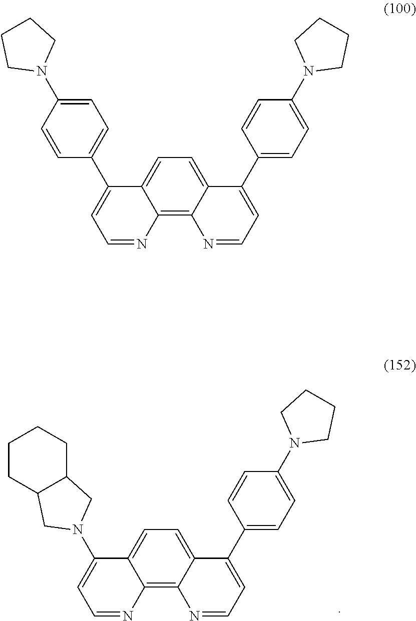 [SMILES: c1cc(-c2ccc(N3CCCC3)cc2)c2ccc3c(-c4ccc(N5CCCC5)cc4)ccnc3c2n1 ; c1cc(-c2ccc(N3CCCC3)cc2)c2ccc3c(N4CC5CCCCC5C4)ccnc3c2n1]

27. A light-emitting device comprising the organic compound according to claim 11.

28. A light-emitting device comprising:
a first electrode, a second electrode, and an organic compound layer between the first electrode and the second electrode,
wherein the organic compound layer comprises a light-emitting layer and an electron-injection layer, and
wherein the electron-injection layer comprises the organic compound according to claim 11.

29. A light-emitting device comprising:
a first electrode, a second electrode, and an organic compound layer between the first electrode and the second electrode,
wherein the organic compound layer comprises a first light-emitting layer, a second light-emitting layer, and an intermediate layer between the first light-emitting layer and the second light-emitting layer, and
wherein the intermediate layer comprises the organic compound according to claim 11.

30. A light-emitting apparatus comprising:
the light-emitting device according to claim 27; and
one of a transistor and a substrate.

31. An electronic appliance comprising:
the light-emitting apparatus according to claim 30; and
any one of a detection portion, an input portion and a communication portion.
